SEQUENCE LISTING (116 sequences)

SEQ ID NO: 1 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 2 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 3 - protein, 255 aa, Homo sapiens [sequence omitted]

SEQ ID NO: 4 - protein, 255 aa, Homo sapiens
MILNKALLLGALALTTVMSPCGGEDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCALGLSVGLMGIVVGTVFIIQGLRSVGASRHQGPL

SEQ ID NO: 5 - protein, 255 aa, Homo sapiens [sequence omitted]

SEQ ID NO: 6 - protein, 255 aa, Homo sapiens [sequence omitted]

SEQ ID NO: 7 - protein, 255 aa, Homo sapiens [sequence omitted]

SEQ ID NO: 8 - protein, 82 aa, Homo sapiens
DHVASCGVNLYQFYGPSGQYTHEFDGDEQFYVDLERKEAAWRWPEFSKFGGFDPQGALRNMAVAKHNLNIMIKRYNSTAATN

SEQ ID NO: 9 - protein, 255 aa, Homo sapiens [sequence omitted]

SEQ ID NO: 10 - protein, 254 aa, Homo sapiens
MILNKALMLGALALTTVMSPCGGEDIVADHVASYGVNLYQSYGPSGQFTHEFDGDEEFYVDLERKETVWKLPLFHRLRFDPQFALTNIAVLKHNLNILIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETVVCALGLSVGLVGIVVGTVLIIRGLRSVGASRHQGPL

SEQ ID NO: 11 - protein, 255 aa, Homo sapiens
MILNKALMLGALALTTVMSPCGGEDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPTPMSELTETVVCALGLSVGLVGIVVGTVLIIRGLRSVGASRHQGPL

SEQ ID NO: 12 - protein, 255 aa, Homo sapiens [sequence omitted]

SEQ ID NO: 13 - protein, 255 aa, Homo sapiens
MILNKALMLGALALTTVMSPCGGEDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSETSFLSKSDHSFFKISYLTFLPSDDEIYDCKVEHWGLDEPLLKHWEPEIPTPMSELTETVVCALGLSVGLVGIVVGTVLIIRGLRSVGASRHQGPL

SEQ ID NO: 14 - protein, 254 aa, Homo sapiens
MILNKALLLGALALTTVMSPCGGEDIVADHVASYGVNLYQSYGPSGQYTHEFDGDEQFYVDLGRKETVWCLPVLRQFRFDPQFALTNIAVTKHNLNILIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETVVCALGLSVGLVGIVVGTVFIIRGLRSVGASRHQGPL

SEQ ID NO: 15 - protein, 175 aa, Homo sapiens
DHVASYGVNLYQSYGPSGQYTHEFDGDEQFYVDLGRKETVWCLPVLRQFRFDPQFALTNIAVTKHNLNILIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHW

SEQ ID NO: 16 - protein, 175 aa, Homo sapiens
DHVASYGVNLYQSYGPSGQYTHEFDGDEQFYVDLGRKETVWCLPVLRQFRFDPQFALTNIAVTKHNLNILIKRSNSTAATNEVPEVTVFSKSPVTLGQPNTLICLVDNIFPPVVNITWLSNGHSVTEGVSEISFLSKSDHSFFKISYLTFLPSADEIYDCKVEHWGLDEPLLKHW

SEQ ID NO: 17 - protein, 175 aa, Homo sapiens
DHVASYGVNLYQSYGPSGQYTHEFDGDEQFYVDLGRKETVWCLPVLRQFRFDPQFALTNIAVTKHNLNILIKRSNSTAATNEVPEVTVFSKSPVTLGQPNTLICLVDNIFPPVVNITWLSNGHSVTEGVSETSFLSKSDHSFFKISHLTFLPSADEIYDCKVEHWGLDEPLLKHW

SEQ ID NO: 18 - protein, 254 aa, Homo sapiens
MILNKALMLGALALTTVMSPCGGEDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 19 - protein, 86 aa, Homo sapiens
EDIVADHVASYGVNLYQSYGPSGQYTHEFDGDEQFYVDLGRKETVWCLPVLRQFRFDPQFALTNIAVLKHNLNSLIKRSNSTAATN

SEQ ID NO: 20 - protein, 74 aa, Homo sapiens
GVNLYQSYGPSGQYTHEFDGDEQFYVDLGRKETVWCLPVLRQFRFDRQFALTNIAVLKHNLNSLIKRSNSTAAT

SEQ ID NO: 21 - protein, 254 aa, Homo sapiens [sequence omitted]

SEQ ID NO: 22 - protein, 81 aa, Homo sapiens
DHVASYGVNLYQSYGLSGQYTHEFDGDEQFYVDLGRKETVWCLPVLRQFRFDPQFALTNIAVLKHNLNSLIKRSNSTAATN

SEQ ID NO: 23 - protein, 254 aa, Homo sapiens
MILNKALMLGTLALTTVMSPCGGEDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 24 - protein, 254 aa, Homo sapiens [sequence omitted]

SEQ ID NO: 25 - protein, 75 aa, Homo sapiens
GVNLYQSYGPSGQFTHEFDGDEQFYVDLGRKETVWCLPVLRQFRFDPQFALTNIAVTKHNLNILIKRSNSTAATN

SEQ ID NO: 26 - protein, 175 aa, Homo sapiens
DHVASYGVNLYQSYGPSGQFTHEFDGDEQFYVDLGRKETVWCLPVLRQFRFDPQFALTNIAVTKHNLNILIKRSNSTAATNEVPEVTVFSKSPVTLGQPNTLICLVDNIFPPVVNITWLSNGHSVTEGVSETRFLSKSDHSFFKISYLTFLPSADEIYDCKVEHWGLDEPLLKHW

SEQ ID NO: 27 - protein, 260 aa, Homo sapiens
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 28 - protein, 74 aa, Homo sapiens
AFVQTHRPTGEFMFEFDEDEMFYVDLDKKETVWHLEEFGQAFSFEAQGGLANIAILNNNLNTLIQRSNHTQATN

SEQ ID NO: 29 - protein, 81 aa, Homo sapiens
DHVSTYAAFVQTHRPTGEFMFEFDEDEMFYVDLDKKETVWHLEEFGQAFSFEAQGGLANIAILNNNLNTLIQRSNHTQATN

SEQ ID NO: 30 - protein, 220 aa, Homo sapiens
AAFVQTHRPTGEFMFEFDDDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 31 - protein, 75 aa, Homo sapiens
AAFVQTHRPTGEFMFEFDEDEMFYVDLDKKETVWHLEEFGQAFSFEAQGGLANIAILNNNLNTLIQRSNHTQAAN

SEQ ID NO: 32 - protein, 81 aa, Homo sapiens
DHVSTYAAFVQTHRPTGEFMFEFDEDEQFYVDLDKKETVWHLEEFGQAFSFEAQGGLANIAILNNNLNTLIQRSNHTQATN

SEQ ID NO: 33 - protein, 80 aa, Homo sapiens
HVSTYAAFVQTHRPTGEFMFEFDEDEMFYVDLDKKETVWHLEEFGQTFSFEAQGGLANIAILNNNLNTLIQRSNHTQATN

SEQ ID NO: 34 - protein, 81 aa, Homo sapiens
DHVSTYAAFVQTHRPTGEFMFEFDDDEMFYVDLDKKETVWHLEEFGRAFSFEAQGGLANIAILNNNLNTLIQRSNHTQATN

SEQ ID NO: 35 - protein, 81 aa, Homo sapiens
DHVSTYAAFVQTHRPTGEFTFEFDEDEMFYVDLDKKETVWHLEEFGQAFSFEAQGGLANIAILNNNLNTLIQRSNHTQATN

SEQ ID NO: 36 - protein, 229 aa, Homo sapiens
IKADHVSTYAAFVQTHRPTGEFMFEFDEDEQFYVDLDKKETVWHLEEFGRAFSFEAQGGLANIAILNNNLNTLIQRSNHTQAANDPPEVTVFPKEPVELGQPNTLICHIDRFFPPVLNVTWLCNGEPVTEGVAESLFLPRTDYSFHKFHYLTFVPSAEDVYDCRVEHWGLDQPLLKHWEAQEPIQMPETTETVLCALGLVLGLVGIIVGTVLIIKSLRSGHDPRAQGPL

SEQ ID NO: 37 - protein, 81 aa, Homo sapiens
DHVSTYAAFVQTHRPTGEFMFEFDEDEQFYVDLDKKETVWHLEEFGRAFSFEAQGGLANIAILNNNLNTLIQRSNHTQAAN

SEQ ID NO: 38 - protein, 74 aa, Homo sapiens
AFVQTHRPTGEFMFEFDEDEQFYVDLDKKETVWHLEEFGRAFSFEAQGGLANIAILNNNLNTLIQRSNHTQAAN

SEQ ID NO: 39 - protein, 86 aa, Homo sapiens
IKADHVSTYAAFVQTHRPTGEFMFEFDEDEQFYVDLDKKETVWHLEEFGRAFSFEAQGGLANIAILNNNLNTLIQRSNHTQAANDP

SEQ ID NO: 40 - protein, 222 aa, Homo sapiens
TYAMFVQTHRPTGEFMFEFDEDEQFYVDLDKKETVWHLEEFGRAFSFEAQGGLANIAILNNNLNTLIQRSNHTQAANDPPEVTVFPKEPVELGQPNTLICHIDRFFPPVLNVTWLCNGEPVTEGVAESLFLPRTDYSFHKFHYLTFVPSAEDVYDCRVEHWGLDQPLLKHWEAQEPIQMPETTETVLCALGLVLGLVGIIVGTVLIIKSLRSGHDPRAQGPL

SEQ ID NO: 41 - protein, 226 aa, Homo sapiens
DHVSTYAMFVQTHRPTGEFMFEFDEDEQFYVDLDKKETVWHLEEFGRAFSFEAQGGLANIAILNNNLNTLIQRSNHTQAANDPPEVTVFPKEPVELGQPNTLICHIDRFFPPVLNVTWLCNGEPVTEGVAESLFLPRTDYSFHKFHYLTFVPSAEDVYDCRVEHWGLDQPLLKHWEAQEPIQMPETTETVLCALGLVLGLVGIIVGTVLIIKSLRSGHDPRAQGPL

SEQ ID NO: 42 - protein, 79 aa, Homo sapiens
VSTYAMFVQTHRPTGEFMFEFDEDEQFYVDLDKKETVWHLEEFGRAFSFEAQGGLANIAILNNNLNTLIQRSNHTQAAN

SEQ ID NO: 43 - protein, 81 aa, Homo sapiens
DHVSTYAAFVQTHRPTGEFMFEFDEDEMFYVDLDKKETVWHLEEFGRAFSFEAQGGLANIAILNNNLNTLIQRSNHTQAAN

SEQ ID NO: 44 - protein, 220 aa, Homo sapiens
AMFVQTHRPTGEFMFEFDEDEMFYVDLDKKETVWHLEEFGQAFSFEAQGGLANIAISNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 45 - protein, 81 aa, Homo sapiens
DHVSTYAMFVQTHRPTGEFMFEFDEDEMFYVDLDKKETVWHLEEFGQAFSFEAQGGLANIAILNNNLNTLIQRSNHTQATN

SEQ ID NO: 46 - protein, 81 aa, Homo sapiens
DHVSTYAMFVQTHRPTGEFMFEFDDDEMFYVDLDKKETVWHLEEFGQAFSFEAQGGLANIAISNNNLNTLIQRSNHTQATN

SEQ ID NO: 47 - protein, 220 aa, Homo sapiens
AAFVQTHRTTGEFMFEFDDDEMFYVDLDKKETVWHLEEFGRAFSFEAQGGLANIAILNNNLNIAIQRSNHTQAANDPPEVTVFPKEAVELGQPNTLICHIDKFFPPVLNVTWLCNGEPVTEGVAESLFLPRTDYSFHKFHYLTFVPSAEDVYDCRVEHWGLDQPLLKHWEAQEPIQMPETAETVLCALGLVLGLVGIIVGTVLIIKSLRSGHDPRAQGPL

SEQ ID NO: 48 - protein, 261 aa, Homo sapiens
MGHEQNQGAALLQMLPLLWLLPHSWAVPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSRGFPIAEVFTLKPLEFGKPNTLVCFVSNLFPPMLTVNWQHHSVPVEGFGPTFVSAVDGLSFQAFSYLNFTPEPSDIFSCIVTHEIDRYTAIAYWVPRNALPSDLLENVLCGVAFGLGVLGIIVGIVLIIYFRKPCSGD

SEQ ID NO: 49 - protein, 250 aa, Homo sapiens
MALRAGLVLGFHTLMTLLSPQEAGATKADHMGSYGPAFYQSYGASGQFTHEFDEEQLFSVDLKKSEAVWRLPEFGDFARFDPQGGLAGIAAIKAHLDILVERSNRSRAINVPPRVTVLPKSRVELGQPNILICIVDNIFPPVINITWLRNGQTVTEGVAQTSFYSQPDHLFRKFHYLPFVPSAEDVYDCQVEHWGLDAPLLRHWELQVPIPPPDAMETLVCALGLAIGLVGFLVGTVLIIMGTYVSSVPR

SEQ ID NO: 50 - protein, 21 aa, Artificial Sequence
  Synthetic peptide - mouse MOG 35-55
MEVGWYRSPFSRVVHLYRNGK

SEQ ID NO: 51 - protein, 21 aa, Artificial Sequence
  Synthetic peptide - human MOG 35-55
MEVGWYRPPFSRVVHLYRNGK

SEQ ID NO: 52 - protein, 20 aa, Artificial Sequence
  Synthetic peptide - human MOG 1-25
GQFRVIGPRHPIRALVGDEV

SEQ ID NO: 53 - protein, 23 aa, Artificial Sequence
  Synthetic peptide - human MOG 94-116
GGFTCFFRDHSYQEEAAMELKVE

SEQ ID NO: 54 - protein, 16 aa, Artificial Sequence
  Synthetic peptide - human MOG 145-160
VFLCLQYRLRGKLRAE

SEQ ID NO: 55 - protein, 15 aa, Artificial Sequence
  Synthetic peptide - human MOG 194-208
LVALIICYNWLHRRL

SEQ ID NO: 56 - protein, 15 aa, Artificial Sequence
  Synthetic peptide - MBP 85-99
ENPVVHFFKNIVTPR

SEQ ID NO: 57 - protein, 20 aa, Artificial Sequence
  Synthetic peptide - MBP 145-164
VDAQGTLSKIFKLGGRDSRS

SEQ ID NO: 58 - protein, 24 aa, Artificial Sequence
  Synthetic peptide - MBP 149-171
GTLSKIFKLGGRDSRSGSGPMARR

SEQ ID NO: 59 - protein, 16 aa, Artificial Sequence
  Synthetic peptide - PLP 139-151
CHCLGKWLGHPDKFVG

SEQ ID NO: 60 - protein, 22 aa, Artificial Sequence
  Synthetic peptide - PLP 95-116
GAVRQIFGDYKTTICGKGLSAT

SEQ ID NO: 61 - protein, 15 aa, Artificial Sequence
  Synthetic peptide - IRBP 1177-1191
ADGSSWEGVGVVPDV

SEQ ID NO: 62 - protein, 20 aa, Artificial Sequence
  Synthetic peptide - arrestin 291-310
NRERRGIALDGKIKHEDTNL

SEQ ID NO: 63 - protein, 32 aa, Artificial Sequence
  Synthetic peptide - phosducin 65-96
KERMSRKMSIQEYELIHQDKEDEGCLRKYRRQ

SEQ ID NO: 64 - protein, 5 aa, Artificial Sequence
  Synthetic peptide - recoverin 48-52
QFQSI

SEQ ID NO: 65 - protein, 7 aa, Artificial Sequence
  Synthetic peptide - recoverin 64-70
KAYAQHV

SEQ ID NO: 66 - protein, 20 aa, Artificial Sequence
  Synthetic peptide - recoverin 62-81
PKAYAQHVFRSFDANSDGTL

SEQ ID NO: 67 - protein, 14 aa, Artificial Sequence
  Synthetic peptide - 149-162
EKRAEKIWASFGKK

SEQ ID NO: 68 - protein, 14 aa, Artificial Sequence
  Synthetic peptide - collagen II 261-274
AGFKGEQGPKGEPG

SEQ ID NO: 69 - protein, 15 aa, Artificial Sequence
  Synthetic peptide - collagen II 259-273
GIAGFKGEQGPKGEP

SEQ ID NO: 70 - protein, 14 aa, Artificial Sequence
  Synthetic peptide - collagen II 257-270
EPGIAGFKGEQGPK

SEQ ID NO: 71 - protein, 14 aa, Artificial Sequence
  Synthetic peptide - modified collagen II 257-270
APGIAGFKAEQAAK

SEQ ID NO: 72 - protein, 20 aa, Artificial Sequence
  Synthetic peptide - fibrinogen-alpha 40-59
VERHQSACKDSDWPFCSDED

SEQ ID NO: 73 - protein, 20 aa, Artificial Sequence
  Synthetic peptide - fibrinogen-alpha 616-625
THSTKRGHAKSRPVRGIHTS

SEQ ID NO: 74 - protein, 13 aa, Artificial Sequence
  Synthetic peptide - fibrinogen-alpha 79-91
QDFTNRINKLKNS

SEQ ID NO: 75 - protein, 20 aa, Artificial Sequence
  Synthetic peptide - fibrinogen-alpha 121-140
NNRDNTYNRVSEDLRSRIEV

SEQ ID NO: 76 - protein, 21 aa, Artificial Sequence
  Synthetic peptide - vimentin 59-79
GVYATRSSAVRLRSSVPGVRL

SEQ ID NO: 77 - protein, 19 aa, Artificial Sequence
  Synthetic peptide - vimentin 26-44
SSRSYVTTSTRTYSLGSAL

SEQ ID NO: 78 - protein, 20 aa, Artificial Sequence
  Synthetic peptide - vimentin 256-275
IDVDVSKPDLTAALRDVRQQ

SEQ ID NO: 79 - protein, 19 aa, Artificial Sequence
  Synthetic peptide - vimentin 415-433
LPNFSSLNLRETNLDSLPL

SEQ ID NO: 80 - protein, 17 aa, Artificial Sequence
  Synthetic peptide - alpha-enolase 5-21
KIHAREIFDSRGNPTVE

SEQ ID NO: 81 - protein, 13 aa, Artificial Sequence
  Synthetic peptide - cartilage glycoprotein-39 259-271
PTFGRSFTLASSE

SEQ ID NO: 82 - protein, 10 aa, Artificial Sequence
  Synthetic peptide - alpha 2-gliadin 61-
PQPELPYPQP

SEQ ID NO: 83 - protein, 20 aa, Artificial Sequence
  Synthetic peptide - alpha 2-gliadin 58-77
LQPFPQPQLPYPQPQLPYPQ

SEQ ID NO: 84 - protein, 15 aa, Artificial Sequence
  Synthetic peptide - insulin B 9-23
SHLVEALYLVCGERG

SEQ ID NO: 85 - protein, 8 aa, Artificial Sequence
  Synthetic peptide - insulin B 16-23
YLVCGERG

SEQ ID NO: 86 - protein, 18 aa, Artificial Sequence
  Peptide binding to pDR2/hMOG35-55
FASFEAQGAKANIAVDKA

SEQ ID NO: 87 - protein, 22 aa, Artificial Sequence
  Peptide binding to pDR2/hMOG35-55
RLEEFASFEAQGAKANIAVDKA

SEQ ID NO: 88 - protein, 18 aa, Artificial Sequence
  Peptide binding to pDR2/hMOG35-55
NGRPVTEGVSETVFLPRD

SEQ ID NO: 89 - protein, 18 aa, Artificial Sequence
  Peptide binding to pDR2/hMOG35-55
LDKLTITSQNLQLESLRM

SEQ ID NO: 90 - protein, 15 aa, Artificial Sequence
  Peptide binding to pDR2/hMOG35-55
NSLEEKKPTEAPPKV

SEQ ID NO: 91 - protein, 17 aa, Artificial Sequence
  Peptide binding to pDR2/hMOG35-55
EPLDMEDLSSGLGVTRQ

SEQ ID NO: 92 - protein, 10 aa, Artificial Sequence
  Peptide binding to pDR2/hMOG35-55
AGLQFPVGRV

SEQ ID NO: 93 - protein, 8 aa, Artificial Sequence
  Peptide binding to pDR2/hMOG35-55
HLQLAIRN

SEQ ID NO: 94 - protein, 15 aa, Artificial Sequence
  Peptide binding to pDR2/hMOG35-55
HLQLAIRNDEELNKL

SEQ ID NO: 95 - protein, 19 aa, Artificial Sequence
  Peptide binding to pDR2/hMOG35-55
VTIQGGVLPNIQAVLLPKK

SEQ ID NO: 96 - protein, 20 aa, Artificial Sequence
  Peptide binding to pDR2/hMOG35-55
VTIQGGVLPNIQAVLLPKKT

SEQ ID NO: 97 - protein, 16 aa, Artificial Sequence
  Peptide binding to pDR2/hMOG35-55
SRKESYSVYVYKVLKQ

SEQ ID NO: 98 - protein, 16 aa, Artificial Sequence
  Peptide binding to pDR2/hMOG35-55
AMGIMNSFVNDIFERI

SEQ ID NO: 99 - protein, 17 aa, Artificial Sequence
  Peptide binding to pDR2/hMOG35-55
EIQTAVRLLLPGELAKH

SEQ ID NO: 100 - protein, 18 aa, Artificial Sequence
  Peptide binding to pDR2/hMOG35-55
LLLPGELAKHAVSEGTKA

SEQ ID NO: 101 - protein, 17 aa, Artificial Sequence
  Peptide binding to pDR2/hMOG35-55
HAVSEGTKAVTKYTSSK

SEQ ID NO: 102 - protein, 12 aa, Artificial Sequence
  Peptide binding to pDR2/hMOG35-55
EIAKDFKTDLRF

SEQ ID NO: 103 - protein, 17 aa, Artificial Sequence
  Peptide binding to pDR2/hMOG35-55
LVGLFEDTNLCAIHAKR

SEQ ID NO: 104 - protein, 7 aa, Artificial Sequence
  Peptide binding to pDR2/hMOG35-55
DIKLARR

SEQ ID NO: 105 - protein, 13 aa, Artificial Sequence
  Peptide binding to pDR2/hMOG35-55
DNIQGITKPAIRR

SEQ ID NO: 106 - protein, 16 aa, Artificial Sequence
  Peptide binding to pDR2/hMOG35-55
RISGLIYEETRGVLKV

SEQ ID NO: 107 - protein, 15 aa, Artificial Sequence
  Peptide binding to pDR2/hMOG35-55
KTVTAMDVVYALKRQ

SEQ ID NO: 108 - protein, 7 aa, Artificial Sequence
  Peptide binding to pDR2/hMOG35-55
TLYGFGG

SEQ ID NO: 109 - protein, 21 aa, Artificial Sequence
  Synthetic peptide - HLA-DRA 38-58
KKETVWRLEEFGRFASFEAQG

SEQ ID NO: 110 - protein, 43 aa, Artificial Sequence
  Partial DR alpha1 sequence
GEFMFDFDGDEIFHVDMAKKETVWRLEEFGRFASFEAQGALAN

SEQ ID NO: 111 - protein, 43 aa, Artificial Sequence; identical to SEQ ID NO: 110
  Partial DR4 alpha1 sequence

SEQ ID NO: 112 - protein, 43 aa, Artificial Sequence
  Partial DP2 alpha1 sequence
GEFMFEFDEDEMFYVDLDKKETVWHLEEFGQAFSFEAQGGLAN

SEQ ID NO: 113 - protein, 44 aa, Artificial Sequence
  Partial DQ2 alpha1 sequence
SGQYTHEFDGDEQFYVDLGRKETVWCLPVLRQFRGFDPQFALTN

SEQ ID NO: 114 - protein, 44 aa, Artificial Sequence
  Partial IAs alpha1 sequence
IGQYTHEFDGDEWFYVDLDKKETIWMLPEFGQLTSFDPQGGLQN

SEQ ID NO: 115 - protein, 44 aa, Artificial Sequence
  Partial IAg7 alpha1 sequence
IGQYTHEFDGDELFYVDLDKKKTVWRLPEFGQLILFEPQGGLQN

SEQ ID NO: 116 - protein, 44 aa, Artificial Sequence
  Partial RT1.B alpha1 sequence
RGQFTHEFDGDEEFYVDLDKKETIWRIPEFGQLTSFDPQGGLQN

(19) United States
(12) Patent Application Publication
(10) Pub. No.: US 2015/0044245 A1
(43) Pub. Date: Feb. 12, 2015

(54) PARTIAL MHC CONSTRUCTS AND METHODS OF USE

(71) Applicants: Oregon Health & Science University, Portland, OR (US); The United States Government as represented by the Department of Veterans Affairs, Washington, DC (US)

(72) Inventors: Arthur A. Vandenbark, Portland, OR (US); Gregory G. Burrows, Portland, OR (US); Roberto Meza-Romero, Hillsboro, OR (US); Gil Benedek, Portland, OR (US); Shayne Andrew, Beaverton, OR (US); Jeffery Mooney, Portland, OR (US)

(21) Appl. No.: 14/370,454

(22) PCT Filed: Jan. 4, 2013
(86) PCT No.: PCT/US2013/020287

§ 371 Date: Jul. 2, 2014

Related U.S. Application Data
(60) Provisional application No. 61/584,045, filed on Jan. 6, 2012.

(51) Int. Cl. C07K 14/705; C12Q 1/68; G01N 33/569

(52) U.S. Cl. CPC C07K 14/705; G01N 33/56977; C12Q 1/6881; G01N 2333/70596; G01N 2500/04; G01N 2500/10
USPC 424/185.1; 530/350; 536/23.5; 435/320.1; 435/7.24; 435/375; 435/6.12

(57) ABSTRACT

Disclosed herein are isolated major histocompatibility complex (MHC) class II α1 domain polypeptides and methods of use. In some embodiments, the isolated polypeptide comprises or consists of an MHC class II α1 domain polypeptide (or portion thereof) and does not include an MHC class II α2, β1, or β2 domain. The disclosed MHC class II α1 domain polypeptides are of use in treating or inhibiting disorders in a subject, such as inflammatory and/or autoimmune disorders. Also disclosed are methods of evaluating efficacy of treatment or optimizing treatment of a subject with a polypeptide including an MHC class II α1 domain polypeptide (or portion thereof) or a polypeptide including an MHC class II α1 domain and β1 domain (such as a β1α1 RTL).

### CROSS REFERENCE TO RELATED APPLICATION

This claims the benefit of U.S. Provisional Application No. 61/584,045, filed Jan. 6, 2012, which is incorporated by reference herein in its entirety.

### ACKNOWLEDGMENT OF GOVERNMENT SUPPORT

This invention was made with government support under grant numbers NS47661, AI43960, and DK0688861 awarded by the National Institutes of Health and Merit Review and Research Enhancement Award Program grants awarded by the Department of Veterans Affairs. The government has certain rights in the invention.

### FIELD

This disclosure relates to partial major histocompatibility complex polypeptides and methods of use, particularly in treating or inhibiting inflammatory or auto-immune disorders.

### BACKGROUND

The initiation of an immune response against a specific antigen in mammals is brought about by presentation of that antigen to T-cells. An antigen is presented to T-cells in the context of a major histocompatibility complex (MHC). MHCs are located on the surface of antigen presenting cells (APCs); the three-dimensional structure of MHCs includes a groove or cleft into which the presented antigen fits. When an appropriate receptor on a T-cell interacts with the MHC/antigen complex on an APC in the presence of necessary co-stimulatory signals, the T-cell is stimulated, triggering various aspects of the well-characterized cascade of immune system activation events, including induction of cytotoxic T-cell function, induction of B-cell function, and stimulation of cytokine production.

The pathology of multiple sclerosis is characterized by an abnormal immune response directed against the central nervous system. In particular, T-lymphocytes reactive against myelin antigens are believed to initiate an inflammatory response within the central nervous system. The resultant inflammatory response includes recruited T-lymphocytes, activated macrophages, B-lymphocytes and plasma cells. Soluble mediators released by these inflammatory cells result in demyelination and, to a lesser extent, axonal degeneration. Similarly, inflammatory responses in many autoimmune disorders result in tissue damage, often of a progressive nature. Despite recent progress, effective treatments of autoimmune and inflammatory disorders are still needed.

### SUMMARY

Disclosed herein are isolated major histocompatibility complex (MHC) class II α1 domain polypeptides and methods of use. In some embodiments, the isolated polypeptide comprises or consists of an MHC class II α1 domain polypeptide (or portion thereof) and does not include an MHC class II α2, β1, or β2 domain. In some examples, the MHC class II α1 domain is covalently linked to an antigenic determinant, for example by a peptide linker, a chemical linker, or a direct covalent bond.

The disclosed MHC class II α1 domain polypeptides are of use in treating or inhibiting disorders in a subject, including inflammatory and/or autoimmune disorders. In some embodiments, the MHC class II α1 domain polypeptide (such as an α1 polypeptide or an α1 polypeptide with an antigenic peptide) or portion thereof is administered to a subject with an inflammatory and/or autoimmune disease or disorder in an amount effective to treat or inhibit the disorder. In some examples, the MHC class II α1 domain polypeptide decreases expression and/or activity of CD74.

Also disclosed are methods of determining or evaluating efficacy of treatment or optimizing treatment of a subject with a polypeptide including an MHC class II α1 domain or a polypeptide including an MHC class II α1 domain (or portion thereof) and β1 domain (such as a β1α1 polypeptide). In some embodiments, the methods include determining CD74 expression or activity in a sample from the subject, comparing the CD74 expression or activity with a control and determining efficacy of the treatment or determining whether the dose of the polypeptide should be adjusted based on CD74 expression and/or activity levels.

The foregoing and other features of the disclosure will become more apparent from the following detailed description, which proceeds with reference to the accompanying figures.

### BRIEF DESCRIPTION OF THE DRAWINGS

In the Figures, and throughout the specification, the following terms are used interchangeably: pDR2/mMOG-35-55 is used interchangeably with RTL342M; pDR2/no peptide is used interchangeably with RTL302-5D; pDR2/MBP-85-99 is used interchangeably with RTL340; and pDR2/hMOG35-55 is used interchangeably with RTL1000.

FIG. 1A is a pair of graphs showing the mean clinical EAE daily scores (left) or the cumulative disease index (CDT; right) of DR*1501-Tg mice immunized with mMOG-35-55 peptide/Complete Freund's Adjuvant (CFA)/Ptx. At disease onset (clinical EAE score of ≧2), mice were treated with vehicle, pDR2/mMOG-35-55, pDR2/no peptide, or pDR2/MBP-85-99 as indicated. Arrows indicate the days on which the mice were treated. *p<0.003 for pDR2/mMOG-35-55 vs. vehicle, pDR2/MBP-85-99, and pDR2/no peptide.

FIG. 1B is a pair of graphs showing the mean clinical EAE daily scores (left) or the CDI (right) of DR*1502-Tg mice immunized with hMOG-35-55 peptide/CFA/Ptx. At disease onset (clinical EAE score of ≧2), mice were treated with vehicle, pDR2/mMOG-35-55, pDR2/no peptide, or pDR2/MBP-85-99 as indicated. Arrows indicate the days on which the mice were treated. *p<0.0023 for pDR2/hMOG-35-55 vs. vehicle, pDR2/MBP-85-99, and pDR2/no peptide.

FIG. 2A is a pair of flow cytometry plots showing the setting of a monocyte gate used to identify monocytes. Monocytes were identified as falling within the region indicated by the circle in a forward scatter/side scatter plot shown in the left panel and propidium iodide (PI)-positive dead cells were excluded as shown in the right panel.

FIG. 2B shows flow cytometry plots of blood collected from naïve DR*1501-Tg mice before and 15 minutes after injection of 500 μg of labeled pDR2, sorted on the monocyte gate and phycoerythrin (PE) stained for expression of CD11b (top row), CD19 (second row), CD11c (third row), and CD3 (bottom row), FITC-stained for binding of labeled pDR2 derivatives (x-axis). The left panel shows binding of a no RTL control, the center panel shows binding of RTL342M (pDR2/mMOG-35-55), and the right panel shows binding of RTL1000 (monomeric pDR2/hMOG-35-55).

FIG. 2C shows flow cytometry plots of blood collected from naïve DR*1501-Tg mice incubated in vitro with 500 μg of labeled pDR2, sorted on the monocyte gate and phycoerythrin (PE) stained for expression of CD11b (top row), CD19 (second row), CD11c (third row), and CD3 (bottom row), FITC-stained for binding of labeled pDR2 derivatives (x-axis). The left panel shows binding of a no RTL control, the center panel shows binding of RTL342M, and the right panel shows binding of RTL1000.

FIG. 2D is a digital image of a GFP⁺ CD11b⁺ cell from a DR*1501/GFP transgenic mouse treated with 10 μg/ml RTL342M labeled with Alexa-546 for 40 minutes and evaluated by fluorescence microscopy.

FIG. 3A is a pair of plots showing the setting of a lymphocyte gate analyzed by forward scatter and side scatter. Lymphocytes were identified as falling within the region indicated by the circle in a forward scatter/side scatter plot shown in the left panel and propidium iodide (PI)-positive dead cells were excluded as shown in the right panel.

FIG. 3B shows flow cytometry plots of blood collected from naïve DR*1501-Tg mice before and 15 minutes after injection of 500 μg of labeled RTL, sorted on the lymphocyte gate and phycoerythrin (PE) stained for expression of CD11b (top row), CD19 (second row), CD11c (third row), and CD3 (bottom row), FITC-stained for binding of labeled pDR2 derivatives (x-axis). The left panel shows binding of a no RTL control, the center panel shows binding of RTL342M, and the right panel shows binding of RTL1000.

FIG. 3C shows flow cytometry plots of blood collected from naïve DR*1501-Tg mice incubated in vitro with 500 μg of labeled RTL, sorted on the lymphocyte gate and phycoerythrin (PE) stained for expression of CD11b (top row), CD19 (second row), CD 11c (third row), and CD3 (bottom row), FITC-stained for binding of labeled RTL derivatives (x-axis). The left panel shows binding of a no RTL control, the center panel shows binding of RTL342M, and the right panel shows binding of RTL1000.

FIG. 4A includes a set of flow cytometry plots (left) of peripheral blood mononuclear cells (PBMC) collected from naïve DR*1501-Tg mice, gated according to the monocyte gate of FIG. 2A and stained for expression of CD11b and binding of FITC-DR2 constructs after 90 minutes incubation with vehicle alone (left panel), the pDR2 specific Fab1b11 (center panel), or the control FabD2 (right panel) and a bar graph (right) that represents the binding of pDR2/mMOG-35-55 normalized to the percent binding of pDR2/mMOG-35-55 in the absence of Fab. *** p<0.0001.

FIG. 4B is a pair of graphs showing daily mean clinical EAE scores (top) and CDI (bottom) of DR*1501-Tg mice with EAE treated daily for three days (arrows indicate days of treatment) at onset of disease with either vehicle, RTL342M, RTL342M+Fab1b11, RTL342M+FabD2, or Fab1b11 alone as indicated. Fab fragments were incubated with pMHC prior to treatment. * p<0.05; *** p<0.0001; # p<0.0005.

FIG. 4C, top panel is a histogram of the mean fluorescent intensity (MFI) of CD74 after pre-incubation of RTL342M with Fab1B11 or control FabD2 or with no RTL or Fab1B11 alone. The bottom panel is a bar graph summarizing the data from the top panel. * p<0.05; ** p<0.005, *** p<0.0005.

FIG. 4D left panel is a line graph of clinical EAE score of DR*1501-Tg mice with mMOG-35-55/CFA/Ptx-induced EAE treated at the onset of clinical signs with 100 μg RTL342M (pDR2/mMOG-35-55), equimolar (10 μg) free mMOG-35-55 peptide or vehicle injected i.v. daily for 5 days. The right panel is a bar graph of the same animals showing cumulative disease index. * p<0.05 (daily scores)** p<0.01 (CDI scores) vs. vehicle treated or free mMOG35-55 treated groups.

FIG. 5A is a line graph of binding of RTL1000 to splenocytes. Naïve spleen cells were incubated with the indicated concentrations of Alexa Fluor 488®-labeled RTL1000. Cells were lysed in 6M urea and the proteins separated by SDS PAGE and quantified by fluorescence densitometry. The data best fit to a 2-binding site model (R²=0.998). K_{D} values of the two binding sites were 2.65 nM for the higher affinity site and 131.0 nM for the lower affinity site.

FIG. 5B is a line graph showing the binding of labeled RTL1000 to whole cells isolated from DR*1501-Tg mice in the presence of increasing amounts of the indicated unlabeled competitor.

FIG. 5C is a line graph showing the binding of labeled RTL1000 to whole cells isolated from MHC class II knockout mice in the presence of increasing amounts of the indicated unlabeled competitor.

FIG. 6 is a pair of digital images of blots of immunoprecipitates of proteins bound to pDR2. Splenocytes from DR*1501-Tg mice were surface biotinylated and lysed with a buffer containing CHAPS. Lysates were incubated with RTL1000, then the DR2-specific monoclonal antibody TU39 was added to the lysate. Immune complexes were washed to remove unbound material and the bound proteins were separated from the immune complexes by boiling in reducing electrophoresis sample buffer for 8-10 minutes. Proteins recovered from immunoprecipitation were separated by SDS-PAGE, blotted onto a filter, and then probed with streptavidin PE (left) or Quantum-Red labeled anti-DR2HK14 antibody (right).

FIG. 7 is a plot showing RTL1000 binding to histone complex as measured by surface Plasmon resonance. Histone complex was bound to a CM5 sensor chip by amine coupling and the indicated concentrations of RTL1000 were bound to the complex. The binding curves were fitted to a 1:1 Langmuir binding model with a drifting baseline (Kₐ=5.74×10³ M⁻¹s⁻¹; Kd=3.3×10⁻³ s⁻¹; RMax=223 RU; Chi²=9.89) to result in an apparent affinity constant K_{D} of 575 nM.

FIG. 8A is a digital image of a Western blot of immunoprecipitates of proteins bound to CD74. Splenocytes from DR*1501-Tg mice were surface biotinylated and lysed. Lysates were mixed with Protein L beads conjugated to an anti-CD74 antibody. These were incubated overnight at 4° C. Proteins were recovered from the immune complexes and blotted and visualized using Streptavidin-PE. M indicates molecular weight markers; T indicates total lysate; CD74 IP indicates immunoprecipitated CD74.

FIG. 8B is a pair of digital images of SDS-PAGE gels of CD74 protein complexes immunoprecipitated from surface biotinylated DR*1501-Tg splenocytes incubated with 25 nmole of the indicated FITC-labeled RTL molecules. The gel was scanned for the presence of FITC.

FIG. 9A is a digital image of an SDS-PAGE gel of CD74 protein complexes immunoprecipitated from surface biotinylated DR*1501-Tg splenocytes incubated with FITC-labeled RTL1000 in the presence of unlabeled DR-α1 and DR2-β1.

FIG. 9B is a graph showing inhibition of binding of RTL1000 by DR-α1 in a dose-dependent manner. FITC-labeled RTL1000 bands on a polyacrylamide gel were detected using a Bio-Rad Imager FX® scanner followed by quantification by densitometry using Quantity One® software. Data were normalized and plotted against the concentration of DR-α1 and the curve was fit to a one-site or two-site competition model using GraphPad Prism® software. The best fit was a one-site competition equation with an EC₅₀ of 447 nM and an R² of 0.956.

FIG. 10A is a plot showing the binding of increasing amounts of FITC labeled RTL1000, RTL342M, RTL340 (pDR2/MBP-85-99), RTL302-5D (monomeric pDR2/no peptide), and DR-α1 when incubated with immuno-adsorbed CD74. Data were analyzed using GraphPad Prism® software for one- or two-site binding with R²>0.95 for all bound ligands.

FIG. 10B is a plot showing the binding of increasing amounts of FITC labeled RTL1000, RTL342M, RTL340, RTL302-5D, and DR-α1 when incubated with enriched MHC class II immuno-adsorbed from CD74 depleted lysate by Protein-L beads conjugated to the L243 monoclonal antibody.

FIG. 11A is a histogram showing expression of CD74 on monocytes in in EAE mice relative to naïve mice. The insert shows the average mean fluorescence intensity of CD74 expression in monocytes from naïve (n=4) and mMOG-35-55 immunized mice with EAE (n=4). ** p<0.01.

FIG. 11B is a bar graph showing CD74 expression in blood and spleen of mice with EAE treated with vehicle or RTL342M (100 μg). * p<0.05.

FIG. 11C is a pair of plots showing CD74 expression in monocytes in the spinal cords of mice treated with vehicle (left) or RTL342M (100 μg; right).

FIG. 12A is a set of plots showing RTL1000 FITC binding (top) and CD74 expression (bottom) in CD11b+ monocytes treated with the indicated concentration of RTL1000.

FIG. 12B is a set of line graphs showing the percentage of CD74 expression and the percentage of binding of the indicated RTL. The bottom right panel is a linear regression analysis of all the constructs showing an inverse correlation between RTL binding and CD74 expression.

FIG. 13A is a set of plots showing RTL binding (top) and CD74 expression (bottom) on CD11b+ human monocytes treated with vehicle (no pMHC treatment).

FIG. 13B is a set of plots showing binding of 1 μg RTL342M (top) and CD74 expression (bottom) on CD11b+ human monocytes.

FIG. 13C is a set of plots showing binding of 1 μg RTL1000 (top) and CD74 expression (bottom) on CD11b+ human monocytes.

FIG. 13D is a set of plots showing binding of 5 μg RTL1000 (top) and CD74 expression (bottom) on CD11b+ human monocytes.

FIG. 13E is a set of plots showing binding of 5 μg RTL340 (top) and CD74 expression (bottom) on CD11b+ human monocytes.

FIG. 13F is a line graph showing linear regression of binding of RTL1000 vs. CD74 expression in human monocytes treated with varying concentrations of RTL1000 in culture prior to analysis. Raw data is shown in FIGS. 13A-E.

FIG. 14A is a line graph showing mean clinical EAE daily scores (left) and a bar graph showing CDI (right) for mice in which EAE was induced by immunization with MBP-85-99. MBP-TCR/DR2-Tg mice were treated for five days following onset of disease (indicated by arrows on graph) with vehicle, RTL342M, RTL1000, RTL340, or RTL302-5D. *p<0.02 for RTL342M vs. vehicle and *** p<0.002 for RTL340 and RTL1000 vs. vehicle for EAE daily mean scores and peak disease (left panel). **p<0.0014 for RTL342M vs. vehicle and ***p<0.001 for RTL340 and RTL1000 vs. vehicle for CDI (right panel).

FIG. 14B is a line graph showing mean clinical EAE daily scores (left) and a bar graph showing CDI (right) for mice in which EAE was induced by immunization with mMOG-35-55. DR*1501 mice with EAE were treated at onset with RTL340 (100 μg), RTL342M (100 μg), RTL302-5D (100 μg, 5 daily treatments), or RTL302-5D (1 mg), or RTL342M (100 μg, 2 daily treatments). The vehicle and untreated groups were not significantly different from each other and were combined. *p<0.04, RTL302-5D (1 mg X2), ***p<0.0001, RTL342M (100 μg X5), #p<0.01, RTL342M (100 μg X2) vs. vehicle/untreated for EAE daily mean scores and peak disease (left panel); ***p<0.0001. RTL342M (100 μg X2 and 5 daily treatments), p<0.013, RTL302-5D (1 mg X2 daily treatments) vs. vehicle/untreated for CDI (right panel).

FIG. 14C is a graph showing the relationship between CD74 levels on CD11b+ cells and the average CDI for the groups of mice shown in FIG. 14B.

FIG. 15 is a line graph showing the clinical EAE scores (top) and a bar graph showing CDI (bottom) of mice treated with either pDR2 with covalently bound mMOG-35-55 (100 μg), pDR2 without peptide (1000 μg), DR-α1 (750 μg), or vehicle alone. Each treatment group consisted of three mice. * p<0.05 pDR2 mMOG-35-55 vs. untreated; # p<0.05 pDR2/no peptide vs. untreated; φ p<0.05 DR-α1 vs. untreated.

FIG. 16 is a line graph showing the clinical EAE scores (top) and a bar graph showing CDI (bottom) of mice treated with either vehicle (untreated), modified pDR2 without peptide (RTL302-5D), modified pDR2 with covalently bound mMOG-35-55 (RTL342M), or DR-α1. Each treatment group consisted of three mice.

FIG. 17 is a series of bar graphs showing expression of CD74 (FIG. 17A), ICAM (FIG. 17B), and CD80 (FIG. 17C) on CD11b+ monocytes from spinal cord of EAE DR2-Tg mice treated as indicated.

FIG. 18A is a graph showing relative expression of ICAM-1 measured by real-time PCR in splenocytes isolated from three naïve DR*1501-Tg mice treated with 10 μg/ml RTL342M or buffer for 1 hour, followed by 10 ng/ml LPS stimulation with or without 100 ng/ml MIF for 1 hour. * p<0.05.

FIG. 18B is a bar graph of mean track speed of GFP⁺CD11b⁺ cells from DR*1501/GFP-Tg mice treated with 50 μg/ml RTL342M for two hours. Ten time-lapse fields (5 untreated and 5 treated fields) were imaged by live fluorescence microscopy. *** p<0.0005.

FIG. 18C is a bar graph of mean track displacement length of cells isolated and treated as described in FIG. 18B. *** p<0.0005.

FIG. 18D is a pair of graphs showing tracks of individual cells (9-11 cells in each field) from four representative fields. Cells were isolated and treated as described in FIG. 18B.

FIG. 19 is an exemplary alignment of a portion of MHC class II a chain amino acid sequences showing the DRA α1 amino acid 38-58 region (boxed). The sequences are as follows: DR2 (SEQ ID NO: 110), DR4 (SEQ ID NO: 111), DP2 (SEQ ID NO: 112), DQ2 (SEQ ID NO: 113), IAs (SEQ ID NO: 114), IAg7 (SEQ ID NO: 115), and RT1.B (SEQ ID NO: 116).

FIG. 20 is an image of a Western blot confirming that RTL constructs do not interfere with anti-CD74 antibody binding to CD74. DR*1501 lysate was pre-incubated with no ligand, 15 μg of RTL342M or DR-α1 for 15 hours at 4° C. The lysates were added to ln-1 Ab adsorbed to protein/beads. Immune complexes were washed with 1% CHAPS/TEN buffer 4 times and then TEN buffer 4 times. Material was eluted from beads by boiling for 7-9 minutes in sample buffer with 2% SDS. Samples were then analyzed in a 10-20% polyacrylamide gel, transferred to PVDF and probed with the anti-CD74 mAb ln-1-FITC to detect CD74.

FIG. 21 is a set of three plots with CD74 expression on the X axis and CD11b expression on the Y axis. DR*1501-Tg mice with mMOG35-55/CFA/Ptx-induced EAE were treated at the onset of clinical signs with 100 μg RTL342M, 500 μg DR2-β1, or vehicle daily for three days. * p<0.05.

FIG. 22 left panel is a line graph showing clinical EAE scores of the mice treated as described in FIG. 21. The right panel is a bar graph of cumulative disease index for the same mice. ** p<0.01, *** p<0.001

FIG. 23 left panel is a line graph showing the clinical EAE scores of DR*1501-Tg mice with mMOG-35-55/CFA/Ptx-induced EAE treated at the onset of clinical signs with vehicle, DR2-β1 (500 μg), DR-α1 (100 μg), DR-α1 (300 μg), DR-α1 (500 μg), DR-α1 (1000 μg), or RTL342M (100 μg). *p<0.05 DRα1 vs vehicle; δ p<0.04 RTL342M, DR-α1 (300 μg), DR-α1 (500 μg), DR-α1 (1000 μg) vs. DR2-β1; # p<0.0003 RTL342M, DR-α1 (300 μg), DR-α1 (500 μg), DR-α1 (1000 μg) vs. vehicle; φ p<0.05 DR-α1 (100 μg) vs. DR2-β1. The right panel is a bar graph showing the cumulative disease index for the same animals. *p<0.05; **p<0.005 DR-α1 (100 μg) vs. vehicle; ***p<0.0005 vs. vehicle.

### SEQUENCE LISTING

Any nucleic acid and amino acid sequences listed herein or in the accompanying sequence listing are shown using standard letter abbreviations for nucleotide bases, and three letter code for amino acids, as defined in 37 C.F.R. §1.822. In at least some cases, only one strand of each nucleic acid sequence is shown, but the complementary strand is understood as included by any reference to the displayed strand.

SEQ ID NOs: 1-49 are amino acid sequences of exemplary MHC class II α chain polypeptides.

SEQ ID NOs: 50-85 are amino acid sequences of exemplary antigenic determinant peptides.

SEQ ID NOs: 86-108 are amino acid sequences of peptides from proteins that bind pDR2/hMOG35-55.

SEQ ID NO: 109 is the amino acid sequence of an MHC DRA residue 38-58 fragment.

SEQ ID NOs: 110-116 are partial amino acid sequences of MHC class II α chain amino acid sequences.

### DETAILED DESCRIPTION

Previously, two domain MHC constructs including covalently linked MHC class II β1 and α1 domains (β1α1 polypeptides, also known as recombinant T cell receptor ligands (RTLs)) have been shown to interact directly with the cognate T cell receptor in the absence of co-stimulatory molecules (McMahan et al., J. Biol. Chem. 278:30961-30970, 2003), serving as partial T cell receptor agonists, and triggering suboptimal downstream signaling and cytokine shifts (Burrows et al., J. Immunol. 167:4386-4395, 2001; Wang et al., J. Immunol. 171:1934-1940, 2003). See, e.g., U.S. Pat. No. 6,270,772; U.S. Pat. Publ. Nos. 2005/0142142 and 2009/0280135; incorporated herein by reference. RTLs including a tethered myelin peptide have previously been successfully utilized to treat mice with experimental autoimmune encephalomyelitis (Burrows, Curr. Drug Targets Inflamm. Allergy 4:185-193, 2005) and in a Phase I trial in multiple sclerosis patients (Yadav et al., Neurol. 74:A293-294, 2010; Offner et al., J. Neuroimmunol. 231:7-14, 2011).

As disclosed herein, it has now surprisingly been found that the two domain RTLs bind to a complex including MHC class II invariant chain (CD74), cell-surface histones, and MHC class II itself. In particular, binding to CD74 involves an interaction between MHC class II α1 domain and CD74 that results in rapid dose-dependent downregulation of CD74 on monocytes. This downregulation is caused by two domain RTLs and the α1 domain alone. In addition, it has been unexpectedly found that treatment with the MHC class II α1 domain alone decreases severity of EAE in mice, indicating that the α1 domain can be used as a therapeutic composition, for example for inflammatory and/or autoimmune disorders.

One advantage of a therapeutic agent including an MHC class II α1 domain, but not other MHC class II domains, is that the need for HLA subtyping of patients prior to treatment is reduced. This is particularly true in the context of HLA-DR, which has a single a chain polypeptide which interacts with all of the DR β chain polypeptides. Therefore, it is believed that a DR α1 domain polypeptide can be administered to all individuals, without the need for HLA typing, as is currently required for treatment with a DR β1 domain-containing polypeptide. For other HLA subtypes, subtyping of only the α domain alleles would be required, rather than subtyping of both α and β alleles. Also, due to its smaller size than β1 domain-containing polypeptides (such as β1α1 polypeptides), the disclosed MHC class II α1 domain polypeptides can more be more quickly and cheaply produced in large quantities and fragments of the α1 domain can be produced as a synthetic peptide, even further increasing the ease and speed of production.

The effectiveness of MHC class II β1α1 polypeptides and α1 domain polypeptides in treating experimental autoimmune encephalomyelitis (EAE) in mice was found to be closely correlated with their unexpected ability to downregulate CD74, as shown in the Examples, below. Thus, CD74 levels (such as CD74 expression or activity) can be utilized as a marker of treatment efficacy of in a subject treated with an MHC class II β1α polypeptide or an MHC class II α1 domain polypeptide. Similarly, CD74 levels can be used to optimize treatment (such as optimizing or adjusting dosage) of a subject with an MHC class II β1α1 polypeptide or an MHC class II α1 domain polypeptide.

### I. Abbreviations

APC antigen presenting cell
CDI cumulative disease index
EAE experimental autoimmune encephalomyelitis
HLA human leukocyte antigen
MBP myelin basic protein
MIF macrophage migration inhibitory factor
MHC major histocompatibility complex
MOG myelin oligodendrocyte glycoprotein
PBMC peripheral blood mononuclear cell
pMHC partial MHC (β1α1) polypeptide
PLP proteolipid protein
RTL recombinant T cell receptor ligand

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.). Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below.

All publications, patent applications, patents, and other references mentioned herein are incorporated by reference in their entirety. All GenBank Accession numbers mentioned herein are incorporated by reference in their entirety as present in GenBank on Jan. 6, 2012. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 8 amino acids (such as about 8-50 or 8-23 amino acids) in a unique spatial conformation. Methods of determining spatial conformation of epitopes include, for example, x-ray crystallography and two-dimensional nuclear magnetic resonance.

An antigen can be a tissue-specific antigen, or a disease-specific antigen. These terms are not exclusive, as a tissue-specific antigen can also be a disease-specific antigen. A tissue-specific antigen is expressed in a limited number of tissues, such as a single tissue. A tissue-specific antigen may be expressed by more than one tissue, such as, but not limited to, an antigen that is expressed in the central or peripheral nervous system.

CD74: Also known as CD74 molecule, major histocompatibility complex, class II invariant chain or E. CD74 is a chaperone regulating antigen presentation for immune response. It is also a cell surface receptor for macrophage migration inhibitory factor (MIF).

Nucleic acid and protein sequences for CD74 are publicly available. For example, GenBank Accession Nos. NM_{—}001025158, NM_{—}004355, and NM_{—}001025159 disclose exemplary human CD74 nucleic acid sequences, and GenBank Accession Nos. NP_{—}001020329, NP_{—}004346, and NP_{—}001020330 disclose exemplary human CD74 amino acid sequences. Similarly, GenBank Accession Nos. NM_{—}001042605 and NM_{—}010545 disclose exemplary mouse Cd74 nucleic acid sequences, and GenBank Accession Nos. NP_{—}001036070 and NP_{—}034675 disclose exemplary mouse Cd74 amino acid sequences. Each of these sequences is incorporated herein by reference as present in GenBank on Jan. 6, 2012.

Conservative variants: A substitution of an amino acid residue for another amino acid residue having similar biochemical properties. “Conservative” amino acid substitutions are those substitutions that do not substantially affect or decrease an activity of an MHC Class II polypeptide, such as an MHC class II α1 polypeptide. A polypeptide can include one or more amino acid substitutions, for example 1-10 conservative substitutions, 2-5 conservative substitutions, 4-9 conservative substitutions, such as 1, 2, 5 or 10 conservative substitutions. Specific, non-limiting examples of a conservative substitution include the following examples:

| Original Amino Acid | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Control: A “control” refers to a sample or standard used for comparison with an experimental sample. In some embodiments, the control is a sample obtained from a healthy subject or population of healthy subjects. In other embodiments, the control is a historical control or standard reference value or range of values (such as a previously tested control sample, such as a group of samples that represent baseline or normal values, such as the level of CD74 expression or activity in healthy subjects). In further examples, the control is from a subject prior to treatment (such as CD74 expression or activity level prior to treatment with an MHC class II β1α1 polypeptide or an MHC class II α1 domain polypeptide).

Domain: A discrete part of an amino acid sequence of a polypeptide or protein that can be equated with a particular function. For example, the α and β polypeptides that constitute a MHC class II molecule are each recognized as having two domains, α1, α2 and β1, β2, respectively. The various domains are typically joined by linking amino acid sequences. In one embodiment, the entire domain sequence is included in a recombinant molecule by extending the sequence to include all or part of the linker or the adjacent domain. For example, when selecting the α1 domain of an MHC class II molecule, the selected sequence may extend from amino acid residue number 1 of the α chain, through the entire α1 domain and include all or part of the linker sequence located at about amino acid residues 76-90 (at the carboxy terminus of the α1 domain, between the α1 and β2 domains). The precise number of amino acids in the various MHC molecule domains varies depending on the species of mammal, as well as between classes of genes within a species. The critical aspect for selection of a sequence for use in a recombinant molecule is the maintenance of the domain function rather than a precise structural definition based on the number of amino acids. One of ordinary skill in the art will appreciate that domain function may be maintained even if somewhat less than the entire amino acid sequence of the selected domain is utilized. For example, a number of amino acids at either the amino or carboxy termini of the α1 domain may be omitted without affecting domain function.

The functional activity of a particular selected domain may be assessed in the context of the MHC class II polypeptides provided by this disclosure (e.g., the α1 or β1α1 polypeptides), for example T cell proliferation and/or CD74 binding assays.

Effective amount: A dose or quantity of a specified compound sufficient to inhibit advancement, or to cause regression of a disease or condition, or which is capable of relieving symptoms caused by the disease or condition. For instance, this can be the amount or dose of a disclosed MHC molecule required to treat or inhibit a disorder, such as an inflammatory and/or autoimmune disorder. In one embodiment, an effective amount is the amount that alone, or together with one or more additional therapeutic agents, induces the desired response in a subject, such as treating or inhibiting an inflammatory or autoimmune disorder or other disease or disorder.

Inflammation: A localized protective response elicited by injury to tissue that serves to sequester the inflammatory agent. Inflammation is orchestrated by a complex biological response of vascular tissues to harmful stimuli, such as pathogens, damaged cells, or irritants. It is a protective attempt by the organism to remove the injurious stimuli as well as initiate the healing process for the tissue. An inflammatory response is characterized by an accumulation of white blood cells, either systemically or locally at the site of inflammation. The inflammatory response may be measured by many methods well known in the art, such as the number of white blood cells, the number of polymorphonuclear neutrophils (PMN), a measure of the degree of PMN activation, such as luminal enhanced-chemiluminescence, or a measure of the amount of cytokines present. A primary inflammation disorder is a disorder that is caused by the inflammation itself. A secondary inflammation disorder is inflammation that is the result of another disorder. Inflammation can lead to a host of inflammatory diseases, including, but not limited to rheumatoid arthritis, osteoarthritis, inflammatory lung disease (including chronic obstructive pulmonary lung disease), inflammatory bowel disease (including ulcerative colitis and Crohn's Disease), periodontal disease, polymyalgia rheumatica, atherosclerosis, systemic lupus erythematosus, systemic sclerosis, Sjogren's Syndrome, asthma, allergic rhinitis, and skin disorders (including dermatomyositis and psoriasis) and the like. Auto-immune disorders which include an inflammatory component (including, but not limited to multiple sclerosis) are also considered to be inflammatory disorders.

Inhibiting or treating a disease: “Inhibiting” a disease refers to inhibiting the full development of a disease, for example in a person who is known to have a predisposition to a disease such as an inflammatory or autoimmune disorder. Inhibition of a disease can span the spectrum from partial inhibition to substantially complete inhibition (prevention) of the disease for example in a subject who has a disease or disorder or is at risk of developing a disease or disorder. In some examples, the term “inhibiting” refers to reducing or delaying the onset or progression of a disease. A subject to be administered with an effective amount of the pharmaceutical compound to inhibit or treat the disease or disorder can be identified by standard diagnosing techniques for such a disorder, for example, basis of family history, or risk factor to develop the disease or disorder. In contrast, “treatment” refers to a therapeutic intervention that ameliorates a sign or symptom of a disease or pathological condition after it has begun to develop.

Isolated: An “isolated” biological component (such as a nucleic acid, peptide or protein) has been substantially separated, produced apart from, or purified away from other biological components in the cell of the organism in which the component occurs, e.g., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell, as well as chemically synthesized nucleic acids.

Linker: A molecule that covalently links two molecules (such as two polypeptides). Linkers (such as a peptide linker or a chemical linker) may be included in the recombinant MHC polypeptides of the present disclosure for example between an α1 domain and an antigenic peptide. Peptide linker sequences, which are generally between 2 and 25 amino acids in length, are well known in the art and include, but are not limited to, the glycine(4)-serine spacer described by Chaudhary et al. (Nature 339:394-397, 1989). Similarly, chemical linkers (such as thiol bonds or crosslinking agents) are well known in the art.

MHC Class II: MHC class II molecules are formed from two non-covalently associated proteins, the α chain and the β chain. The α chain comprises α1 and α2 domains, and the β chain comprises β1 and β2 domains. The cleft into which the antigen fits is formed by the interaction of the α1 and β1 domains. The α2 and β2 domains are transmembrane Ig-fold like domains that anchor the α and β chains into the cell membrane of the APC. MHC class II complexes, when associated with antigen (and in the presence of appropriate co-stimulatory signals) stimulate CD4 T-cells. The primary functions of CD4 T-cells are to initiate the inflammatory response, to regulate other cells in the immune system, and to provide help to B cells for antibody synthesis.

Pharmaceutically acceptable carriers: The pharmaceutically acceptable carriers useful in this disclosure are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of the proteins herein disclosed.

Polypeptide: A polymer in which the monomers are amino acid residues which are joined together through amide bonds. When the amino acids are alpha-amino acids, either the L-optical isomer or the D-optical isomer can be used, the L-isomers being preferred. The terms “polypeptide” or “protein” or “peptide” as used herein are intended to encompass any amino acid sequence and include modified sequences such as glycoproteins. The term “polypeptide” or “protein” or “peptide” is specifically intended to cover naturally occurring proteins, as well as those which are recombinantly or synthetically produced. It should be noted that the term “polypeptide” or “protein” includes naturally occurring modified forms of the proteins, such as glycosylated, phosphorylated, or ubiquinated forms.

Purified: The term purified does not require absolute purity; rather, it is intended as a relative term. Thus, for example, a purified peptide preparation is one in which the peptide or protein is more enriched than the peptide or protein is in its environment, for example within a cell or in a preparation. Preferably, a preparation is purified such that the protein or peptide represents at least 50% of the total peptide or protein content of the preparation. In some embodiments, a purified preparation contains at least 60%, at least 70%, at least 80%, at least 85%, at least 90%, at least 95% or more of the protein or peptide.

Recombinant: A recombinant nucleic acid or polypeptide is one that has a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques.

Sample: A biological specimen containing nucleic acid (e.g., DNA or RNA (including mRNA)), protein, or combinations thereof, obtained from a subject. Examples include, but are not limited to, peripheral blood, fine needle aspirate, urine, saliva, tissue biopsy, surgical specimen, and autopsy material. In one example, a sample includes a blood sample (such as blood; derivatives and fractions of blood, such as serum) or isolated or purified cell populations (for example, T cells, B cells, PBMC, lymphocytes, and so on, including partially isolated or partially purified cell populations).

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith & Waterman, Adv. Appl. Math. 2: 482, 1981; Needleman & Wunsch, J. Mol. Biol. 48: 443, 1970; Pearson & Lipman, Proc. Natl. Acad. Sci. USA 85: 2444, 1988; Higgins & Sharp, Gene, 73: 237-244, 1988; Higgins & Sharp, Comput. Appl. Biosci. 5: 151-153, 1989; Corpet et al., Nucl. Acids Res. 16, 10881-90, 1988; Huang et al., Comput. Appl. Biosci. 8, 155-65, 1992; and Pearson, Methods Mol. Biol. 24:307-331, 1994. Altschul et al. (J. Mol. Biol. 215:403-410, 1990) presents a detailed consideration of sequence alignment methods and homology calculations. The NCBI Basic Local Alignment Search Tool (BLAST) (Altschul et al., J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx.

Nucleic acid sequences that do not show a high degree of sequence identity may nevertheless encode similar amino acid sequences, due to the degeneracy of the genetic code. It is understood that changes in nucleic acid sequence can be made using this degeneracy to produce multiple nucleic acid molecules that all encode substantially the same protein.

Subject: Living multi-cellular vertebrate organisms, a category that includes both human and non-human mammals.

### III. MHC Class II α1 Polypeptides

Disclosed herein are isolated polypeptides which include an MHC class II α1 domain or fragment thereof and do not include MHC class II α2, β1, or β2 domains. The amino acid sequences of mammalian MHC class II α and β chain proteins, as well as nucleic acids encoding these proteins, are well known in the art and available from numerous sources including GenBank. Exemplary sequences are provided in Auffray et al. (Nature 308:327-333, 1984) (human HLA DQ α); Larhammar et al. (Proc. Natl. Acad. Sci. USA 80:7313-7317, 1983) (human HLA DQ β); Das et al. (Proc. Natl. Acad. Sci. USA 80:3543-3547, 1983) (human HLA DR α); Tonnelle et al. (EMBO J. 4:2839-2847, 1985) (human HLA DR (3); Lawrance et al. (Nucl. Acids Res. 13:7515-7528, 1985) (human HLA DP α); Kelly and Trowsdale (Nucl. Acids Res. 13:1607-1621, 1985) (human HLA DP β); Syha et al. (Nucl. Acids Res. 17:3985, 1989) (rat RT1.B α); Syha-Jedelhauser et al. (Biochim. Biophys. Acta 1089:414-416, 1991) (rat RT1.B β); Benoist et al. (Proc. Natl. Acad. Sci. USA 80:534-538, 1983) (mouse I-A α); Estess et al. (Proc. Natl. Acad. Sci. USA 83:3594-3598, 1986) (mouse I-A β), all of which are incorporated by reference herein. Additional MHC class II α and β chain polypeptides can be identified by one of ordinary skill in the art, for example, from public databases, such as the IMGT/HLA database (available on the world wide web at ebi.ac.uk/imgt/hla/).

In some embodiments, the disclosed polypeptides include an MHC class II α1 domain, such as a DR-α1, DP-α1, DQ-α1, DM-α1, or DO-α1 domain, or a portion thereof. In a particular embodiment, the MHC class II α1 domain is a human HLA-DRA polypeptide. The α1 domain is well defined in mammalian MHC class II α chain proteins. In some examples, MHC class II α chains include a leader sequence that is involved in trafficking the polypeptide and is proteolytically removed to produce the mature α polypeptide. The α1 domain is generally regarded as comprising about residues 1-90 of the mature (proteolytically processed) a chain. The native peptide linker region between the α1 and α2 domains of the MHC class II protein spans from about amino acid 76 to about amino acid 93 of the mature α chain, depending on the particular a chain under consideration. Exemplary MHC class II a polypeptides are provided herein (e.g., SEQ ID NOs: 1-49). Thus, an α1 domain may include about amino acid residues 1-90 of the mature a chain, but one of ordinary skill in the art will recognize that the C-terminal cut-off of this domain is not necessarily precisely defined, and, for example, might occur at any point between amino acid residues 70-100 of the mature a chain. In some examples, the α1 domain includes amino acids 1-70, 1-71, 1-72, 1-73, 1-74, 1-75, 1-76, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, or 1-100 of a mature MHC class II α domain. In other examples, an α1 domain includes about residues 20-120 (such as about residues 20-110, 24-110, 24-109, 25-100, 25-109, 26-110, 26-109, 30-120, 32-120, 32-115, 26-90, 26-85, 26-84, or other overlapping regions) of a full length MHC class II α polypeptide (such as SEQ ID NOs: 1-49 disclosed herein). In some examples, the MHC class II α1 domain does not include an N-terminal methionine; however, an N-terminal methionine can be present, for example as a result of expression in a bacterial, yeast, or mammalian system.

In further examples, the α1 domain can include deletion or addition of a few amino acids at the 5′- and/or 3′-end, such as addition or deletion of about 1-10 amino acids, such as addition or deletion of 1, 2, 3, 4, 5, 6, 7, 8, 9, or 10 amino acids from the 5′- or 3′-end, or combinations thereof (such as a deletion from one end and an addition to the other end). The composition of the α1 domain may also vary outside of these parameters depending on the mammalian species and the particular a chain in question. One of ordinary skill in the art will appreciate that the precise numerical parameters of the amino acid sequence are less important than the maintenance of domain function (for example, CD74 binding or downregulation).

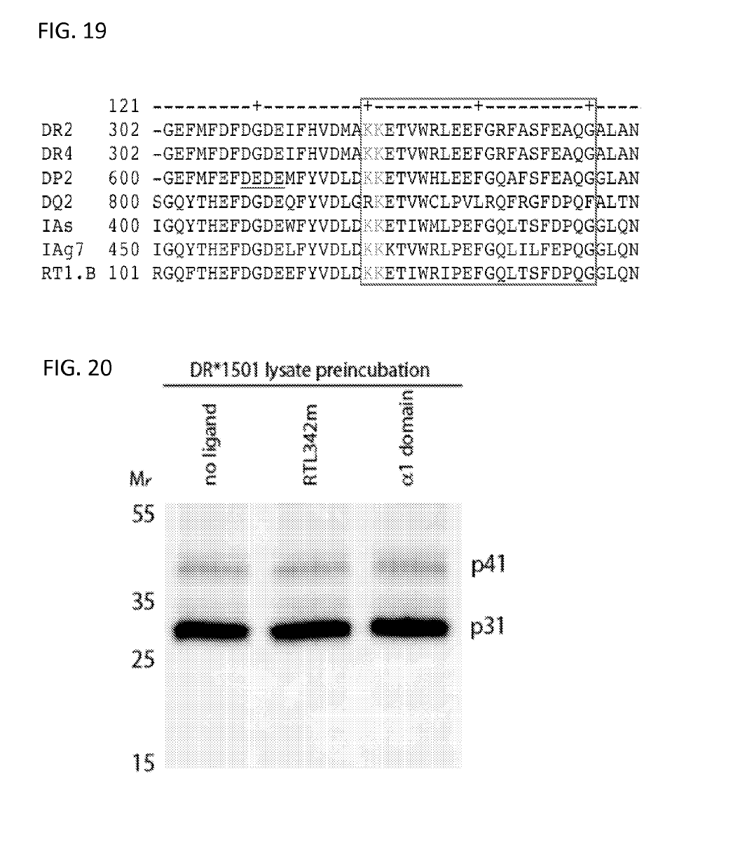

In other examples, the α1 domain polypeptide includes or consists of a portion of the α1 domain, such as a portion of the α1 domain capable of binding to and/or decreasing expression or activity of CD74. For example, the MHC class II α1 polypeptide can include or consist of amino acids 38-58 of a mature HLA-DRA polypeptide (for example, KKETVWRLEEFGRFASFEAQG; SEQ ID NO: 109) or a portion thereof (such as 5 or more contiguous amino acids, for example, 5-16, 8-15, 8-10, or 12-15 contiguous amino acids thereof) or a homologous region of an HLA-DP, HLA-DQ, HLA-DM, or HLA-DO polypeptide. An exemplary alignment is shown in FIG. 19. One of ordinary skill in the art can identify homologous regions of other MHC class II a domain polypeptides, for example utilizing multiple sequence alignment tools.

In several embodiments, an MHC class II α polypeptide includes or consists of the amino acid sequence set forth as SEQ ID NOs: 1-49. In additional embodiments, an MHC class II a polypeptide has a sequence at least 75%, 85%, 90%, 95%, 96%, 97%, 98% or 99% identical to the amino acid sequence set forth in one of SEQ ID NOs: 1-49 or a fragment thereof. For example, the polypeptide can have an amino acid sequence at least 85%, 90%, 95%, 96%, 97%, 98% or 99% identical to one of the amino acid sequences set forth in SEQ ID NOs: 1-49 or a fragment thereof. Exemplary sequences can be obtained using computer programs that are readily available on the internet and the amino acid sequences set forth herein. In some examples, the polypeptide retains a function of the MHC class II α1 polypeptide, such as binding to CD74. Exemplary MHC class II α1 domain polypeptides include those shown in Table 1.

TABLE 1
Exemplary MHC class II α1 domain polypeptides

| MHC Class II α Chain | α1 Domain Residues |
|---|---|
| DRA*0101 | 26-109 of SEQ ID NO: 1 |
| DRA*010202 | 26-109 of SEQ ID NO: 2 |
| DQA1*010101 | 24-110 of SEQ ID NO: 3 |
| DQA1*010201 | 24-110 of SEQ ID NO: 4 |
| DQA1*010301 | 24-110 of SEQ ID NO: 5 |
| DQA1*010401 | 24-110 of SEQ ID NO: 6 |
| DQA1*0105 | 24-110 of SEQ ID NO: 7 |
| DQA1*0106 | 1-82 of SEQ ID NO: 8 |
| DQA1*0107 | 24-110 of SEQ ID NO: 9 |
| DQA1*0201 | 24-109 of SEQ ID NO: 10 |
| DQA1*030101 | 24-110 of SEQ ID NO: 11 |
| DQA1*0302 | 24-110 of SEQ ID NO: 12 |
| DQA1*0303 | 24-110 of SEQ ID NO: 13 |
| DQA1*040101 | 24-109 of SEQ ID NO: 14 |
| DQA1*040102 | 1-81 of SEQ ID NO: 15 |
| DQA1*0402 | 1-81 of SEQ ID NO: 16 |
| DQA1*0404 | 1-81 of SEQ ID NO: 17 |
| DQA1*050101 | 24-109 of SEQ ID NO: 18 |
| DQA1*050102 | 1-86 of SEQ ID NO: 19 |
| DQA1*0502 | 1-74 of SEQ ID NO: 20 |
| DQA1*0503 | 24-109 of SEQ ID NO: 21 |
| DQA1*0504 | 1-81 of SEQ ID NO: 22 |
| DQA1*0505 | 24-109 of SEQ ID NO: 23 |
| DQA1*060101 | 24-109 of SEQ ID NO: 24 |
| DQA1*060102 | 1-75 of SEQ ID NO: 25 |
| DQA1*0602 | 1-81 of SEQ ID NO: 26 |
| DPA1*010301 | 32-115 of SEQ ID NO: 27 |
| DPA1*010302 | 1-74 of SEQ ID NO: 28 |
| DPA1*010303 | 1-81 of SEQ ID NO: 29 |
| DPA1*0104 | 1-75 of SEQ ID NO: 30 |
| DPA1*0105 | 1-75 of SEQ ID NO: 31 |
| DPA1*010601 | 1-81 of SEQ ID NO: 32 |
| DPA1*0107 | 1-80 of SEQ ID NO: 33 |
| DPA1*0108 | 1-81 of SEQ ID NO: 34 |
| DPA1*0109 | 1-81 of SEQ ID NO: 35 |
| DPA1*020101 | 1-84 of SEQ ID NO: 36 |
| DPA1*020102 | 1-81 of SEQ ID NO: 37 |
| DPA1*020104 | 1-74 of SEQ ID NO: 38 |
| DPA1*020106 | 1-84 of SEQ ID NO: 39 |
| DPA1*020201 | 1-77 of SEQ ID NO: 40 |
| DPA1*020202 | 1-81 of SEQ ID NO: 41 |
| DPA1*020203 | 1-79 of SEQ ID NO: 42 |
| DPA1*0203 | 1-81 of SEQ ID NO: 43 |
| DPA1*0301 | 1-75 of SEQ ID NO: 44 |
| DPA1*0302 | 1-81 of SEQ ID NO: 45 |
| DPA1*0303 | 1-81 of SEQ ID NO: 46 |
| DPA1*0401 | 1-81 of SEQ ID NO: 47 |
| DOA*010101 | 26-84 of SEQ ID NO: 49 |

Minor modifications of an MHC class II α1 polypeptide primary amino acid sequences may result in peptides which have substantially equivalent activity as compared to the unmodified counterpart polypeptide described herein. Such modifications may be deliberate, as by site-directed mutagenesis, or may be spontaneous. All of the polypeptides produced by these modifications are included herein. Thus, a specific, non-limiting example of an MHC class II α1 polypeptide is a conservative variant of an α1 polypeptide (such as a conservative amino acid substitution, for example, one or more conservative amino acid substitutions, for example 1-10 conservative substitutions, 2-5 conservative substitutions, 4-9 conservative substitutions, such as 1, 2, 5 or 10 conservative substitutions). A list of exemplary conservative substitutions is provided above. Substitutions of amino acid sequences, such as those shown in SEQ ID NOs: 1-49 or fragments thereof, can be made based on this list.

Nucleic acid molecules encoding the α1 domain may be produced by standard means, such as amplification by the polymerase chain reaction (PCR). Standard approaches for designing primers for amplifying open reading frames encoding the α1 domain may be employed. Libraries suitable for the amplification of the α1 domain include, for example, cDNA libraries prepared from the mammalian species in question; such libraries are available commercially, or may be prepared by standard methods. Thus, for example, constructs encoding an α1 domain polypeptides may be produced by PCR using primers corresponding to the 5′ and 3′ ends of the α1 domain coding region. Following PCR amplification, the amplified nucleic acid molecule may be cloned into a standard cloning vector. In some embodiments, for example to facilitate convenient cloning or linkage of an antigenic determinant (discussed below), one or both of the primers used to amplify an α1 domain may include a suitable restriction enzyme site such that the α1 domain encoding fragment may be readily ligated with another nucleic acid following amplification and digestion with the selected restriction enzyme.

In some embodiments, the MHC class II α1 domain polypeptide is expressed in prokaryotic or eukaryotic cells from a nucleic acid construct. Nucleic acid constructs expressing the MHC class II α1 domain polypeptide may also include regulatory elements such as promoters, enhancers, and 3′ regulatory regions, the selection of which will be determined based upon the type of cell in which the protein is to be expressed. The constructs are introduced into a vector suitable for expressing the MHC class II α1 domain polypeptide in the selected cell type.

Numerous prokaryotic and eukaryotic systems are known for the expression and purification of polypeptides. For example, heterologous polypeptides can be produced in prokaryotic cells by placing a strong, regulated promoter and an efficient ribosome binding site upstream of the polypeptide-encoding construct. Suitable promoter sequences include the beta-lactamase, tryptophan (trp), phage T7 and lambda P_{L} promoters. Methods and plasmid vectors for producing heterologous proteins in bacteria or mammalian cells are described in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); and Ausubel et al., Short Protocols in Molecular Biology: A Compendium of Methods from Current Protocols in Molecular Biology, 4th ed., Wiley & Sons, 1999.

Suitable prokaryotic cells for expression of large amounts of proteins include Escherichia coli and Bacillus subtilis. Often, proteins expressed at high levels are found in insoluble inclusion bodies; methods for extracting proteins from these aggregates are described for example, by Sambrook et al. (2001, see chapter 15). Recombinant expression of MHC class II α1 domain polypeptides in prokaryotic cells may alternatively be conveniently obtained using commercial systems designed for optimal expression and purification of fusion proteins. Such fusion proteins typically include a tag that facilitates purification. Examples of such systems include: the pMAL protein fusion and purification system (New England Biolabs, Inc., Beverly, Mass.); the GST gene fusion system (Amersham Pharmacia Biotech, Inc., Piscataway, N.J.); and the pTrcHis expression vector system (Invitrogen, Carlsbad, Calif.). Additional systems include the His6-tag (e.g., Roche Applied Science, Mannheim, Germany) or streptavidin binding peptide (e.g., Sigma-Aldrich, St. Louis, Mo.). For example, the pMAL expression system utilizes a vector that adds a maltose binding protein to the expressed protein. The fusion protein is expressed in E. coli. and the fusion protein is purified from a crude cell extract using an amylose column. If necessary, the maltose binding protein domain can be cleaved from the fusion protein by treatment with a suitable protease, such as Factor Xa. The maltose binding fragment can then be removed from the preparation by passage over a second amylose column.

The MHC class II α1 domain polypeptides can also be expressed in eukaryotic expression systems, including Pichia pastoris, Drosophila. Baculovirus and Sindbis expression systems produced by Invitrogen (Carlsbad, Calif.). Eukaryotic cells such as Chinese Hamster ovary (CHO), monkey kidney (COS), HeLa, Spodoptera frugiperda, and Saccharomyces cerevisiae may also be used to express the MHC class II α1 domain polypeptides. Regulatory regions suitable for use in these cells include, for mammalian cells, viral promoters such as those from CMV, adenovirus or SV40, and for yeast cells, the promoter for 3-phosphoglycerate kinase or alcohol dehydrogenase.

The transfer of DNA into eukaryotic cells is routine. The vectors are introduced into the recipient cells as pure DNA (transfection) by, for example, precipitation with calcium phosphate or strontium phosphate, electroporation, lipofection, DEAE dextran, microinjection, protoplast fusion, or microprojectile guns. Alternatively, the nucleic acid molecules can be introduced by infection with virus vectors. Systems are developed that use, for example, retroviruses, adenoviruses, or Herpes virus.

An MHC class II α1 domain polypeptide produced in mammalian cells may be extracted following release of the protein into the supernatant and may be purified using an immunoaffinity column prepared using anti-MHC antibodies. Alternatively, the MHC polypeptide may be expressed as a chimeric protein with, for example, β-globin. Antibody to β-globin is thereafter used to purify the chimeric protein. Corresponding protease cleavage sites engineered between the β-globin gene and the nucleic acid sequence encoding the MHC class II α1 domain polypeptide are then used to separate the two polypeptide fragments from one another after translation. One useful expression vector for generating β-globin chimeric proteins is pSG5 (Stratagene, La Jolla, Calif.).

Expression of the MHC polypeptides in prokaryotic cells will result in polypeptides that are not glycosylated. Glycosylation of the polypeptides at naturally occurring glycosylation target sites may be achieved by expression of the polypeptides in suitable eukaryotic expression systems, such as mammalian cells. In other examples, the MHC class II α1 domain can be modified (for example, utilizing site-directed mutagenesis) to include desired post-translational modification sites, such as one or more sites for N-linked glycosylation, phosphorylation, or other modifications.

Purification of the expressed protein is generally performed in a basic solution (typically around pH 10) containing 6M urea. Folding of the purified protein is then achieved by dialysis against a buffered solution at neutral pH (typically phosphate buffered saline at around pH 7.4).

### IV. Antigenic Determinants

In some embodiments, the disclosed methods utilize MHC class II molecules (such as an MHC class II α1 domain) including a covalently linked antigenic determinant. As is well known in the art (see for example U.S. Pat. No. 5,468,481) the presentation of antigen in MHC complexes on the surface of APCs generally does not involve a whole antigenic peptide. Rather, a peptide located in the groove between the β1 and α1 domains (in the case of MHC II) or the α1 and α2 domains (in the case of MHC I) is typically a small fragment of the whole antigenic peptide. As discussed in Janeway & Travers (Immunobiology: The Immune System in Health and Disease, 1997), peptides located in the peptide groove of MHC class I molecules are constrained by the size of the binding pocket and are typically 8-15 amino acids long (such as 8, 9, 10, 11, 12, 13, 14, or 15 amino acids), more typically 8-10 amino acids in length (but see Collins et al., Nature 371:626-629, 1994 for possible exceptions). In contrast, peptides located in the peptide groove of MHC class II molecules are not constrained in this way and are often larger, typically at least 3-50 amino acids in length (such as 8-30, 10-25, or 15-23 amino acids in length). In some examples, the peptide located in the peptide groove of an MHC class II molecule is about 15-23 amino acids in length. In some examples, the disclosed compositions include an antigenic peptide, for example, an antigenic peptide covalently linked to an MHC class II α1 domain. Peptide fragments can be prepared by standard means, such as use of synthetic peptide synthesis machines.

In some examples, an antigenic determinant includes a peptide from a neuronal or central nervous system protein, such as a myelin protein (for example, myelin oligodendrocyte glycoprotein (MOG), myelin basic protein (MBP), or proteolipid protein (PLP)). In other examples, an antigenic determinant is a peptide from a retinal protein, such as interphotoreceptor retinoid binding protein (IRBP), arrestin, phosducin, or recoverin. Additional antigenic determinants include peptides from type II collagen (collagen II), fibrinogen-α, vimentin, α-enolase, human cartilage glycoprotein-39, α2 gliadin, or insulin. In some examples, an antigenic determinant includes a post-translational modification, such as phosphorylation, glycosylation, or citrullination. Exemplary antigenic peptides are provided in Table 2. One of ordinary skill in the art can identify additional antigenic determinants relevant to a particular disease or disorder.

TABLE 2
Exemplary antigenic determinants

| Antigenic | | SEQ |
|---|---|---|
| Determinant Peptide | Peptide Sequence | ID NO: |
| mMOG 35-55 | MEVGWYRSPFSRVVHLYRNGK | 50 |
| hMOG 35-55 | MEVGWYRPPFSRVVHLYRNGK | 51 |
| MOG 1-25 | GQFRVIGPRHPIRALVGDEV | 52 |
| MOG 94-116 | GGFTCFFRDHSYQEEAAMELKVE | 53 |
| MOG 145-160 | VFLCLQYRLRGKLRAE | 54 |
| MOG 194-208 | LVALIICYNWLHRRL | 55 |
| MBP85-99 | ENPVVHFFKNIVTPR | 56 |
| MBP 145-164 | VDAQGTLSKIFKLGGRDSRS | 57 |
| MBP 149-171 | GTLSKIFKLGGRDSRSGSGPMAR | 58 |
| | R | |
| PLP 139-151 | CHCLGKWLGHPDKFVG | 59 |
| PLP 95-116 | GAVRQIFGDYKTTICGKGLSAT | 60 |
| IRBP 1177-1191 | ADGSSWEGVGVVPDV | 61 |
| Arrestin 291-310 | NRERRGIALDGKIKHEDTNL | 62 |
| Phosducin 65-96 | KERMSRKMSIQEYELIHQDKEDE | 63 |
| | GCLRKYRRQ | |
| Recoverin 48-52 | QFQSI | 64 |
| Recoverin 64-70 | KAYAQHV | 65 |
| Recoverin 62-81 | PKAYAQHVFRSFDANSDGTL | 66 |
| Recoverin 149-162 | EKRAEKIWASFGKK | 67 |
| Collagen II 261-274 | AGFKGEQGPKGEPG | 68 |
| Collagen II 259-273 | GIAGFKGEQGPKGEP | 69 |
| Collagen II 257-270 | EPGIAGFKGEQGPK | 70 |
| Modified collagen II | APGIAGFKAEQAAK | 71 |
| 257-270 | | |
| Fibrinogen-α 40-59 | VERHQSACKDSDWPFCSDED | 72 |
| Fibrinogen-α 616-625 | THSTKRGHAKSRPVRGIHTS | 73 |
| Fibrinogen-α79-91 | QDFTNRINKLKNS | 74 |
| Fibrinogen-α 121-140 | NNRDNTYNRVSEDLRSRIEV | 75 |
| Vimentin 59-79 | GVYATRSSAVRLRSSVPGVRL | 76 |
| Vimentin 26-44 | SSRSYVTTSTRTYSLGSAL | 77 |
| Vimentin 256-275 | IDVDVSKPDLTAALRDVRQQ | 78 |
| Vimentin 415-433 | LPNFSSLSLRETNLDSLPL | 79 |
| α-enolase 5-21 | KIHAREIFDSRGNPTVE | 80 |
| Cartilage | PTFGRSFTLASSE | 81 |
| glycoprotein-39 | | |
| 259-271 | | |
| α2-gliadin 61-71 | PQPELPYPQP | 82 |
| α2-gliadin 58-77 | LQPFPQPQLPYPQPQLPYPQ | 83 |
| Insulin B 9-23 | SHLVEALYLVCGERG | 84 |
| Insulin B 16-23 | YLVCGERG | 85 |

In some examples, the antigenic peptide is covalently linked to the MHC class II ad polypeptide by operably linking a nucleic acid sequence encoding the selected antigen to the 5′ end of the construct encoding the MHC class II α1 polypeptide such that, in the expressed peptide, the antigenic peptide is linked to the amino-terminus of the α1 domain. In other examples, the antigenic peptide is covalently linked to the MHC class II α1 polypeptide by operably linking a nucleic acid sequence encoding the selected antigen to the 3′ end of the construct encoding the MHC class II ad polypeptide such that, in the expressed peptide, the antigenic peptide is linked to the carboxy-terminus of the α1 domain. One convenient way of obtaining this result is to incorporate a sequence encoding the antigen into the PCR primers used to amplify the MHC class II α1 domain coding regions. In some examples, a sequence encoding a linker peptide sequence is included between the antigenic peptide and the MHC class II α1 polypeptide. However, it is not necessary that the antigenic peptide be ligated exactly at the 5′ end (or 3′ end) of the MHC class II α1 domain coding region. For example, the antigenic coding region may be inserted within the first few (typically within the first 10) codons of the 5′ or 3′ end of the MHC class II α1 domain coding sequence.

In some embodiments, this genetic system for linkage of the antigenic peptide to the MHC class II α1 domain is particularly useful where a number of MHC class II α1 domains with differing antigenic peptides are to be produced. The described system permits the construction of an expression vector in which a unique restriction site is included in the MHC class II α1 domain (e.g., at the 5′ or 3′ end of the α1 domain). In conjunction with such a construct, a library of antigenic peptide-encoding sequences is made, with each antigen-coding region flanked by sites for the selected restriction enzyme. The inclusion of a particular antigen into the MHC class II α1 domain is then performed simply by (a) releasing the antigen-coding region with the selected restriction enzyme, (b) cleaving the MHC class II α1 domain construct with the same restriction enzyme, and (c) ligating the antigen coding region into the MHC class II α1 domain construct. In this manner, a large number of MHC class II α1 domain-peptide antigen constructs can be made and expressed in a short period of time.

In some examples, the antigen is covalently linked to the MHC class II α1 domain polypeptide by a disulfide bond. In some examples, the disulfide linkage is formed utilizing a naturally occurring cysteine residue in the MHC class II α1 domain polypeptide (such as a cysteine residue in the MHC class II α1 domain). One of ordinary skill in the art can identify a suitable cysteine residue in an MHC class II α1 domain polypeptide. In other examples, the disulfide linkage is formed utilizing a non-naturally occurring cysteine residue in the MHC class II α1 domain polypeptide, such as a cysteine residue introduced in the MHC class II α1 domain polypeptide by mutagenesis. In further examples, the disulfide linkage is formed utilizing a naturally occurring cysteine residue in the peptide antigen. In still further examples, the disulfide linkage is formed utilizing a non-naturally occurring cysteine residue in the peptide antigen, such as a cysteine residue introduced in the peptide antigen by mutagenesis.

### V. Methods of Treating or Inhibiting Disorders

Disclosed herein are methods of treating or inhibiting disorders in a subject, including but not limited to inflammatory and/or autoimmune disorders. The disclosed methods include administering an MHC class II α1 domain polypeptide (such as an α1 domain polypeptide or an α1 domain polypeptide covalently linked to an antigen) to a subject.

In some embodiments, the methods include selecting a subject with a disorder for treatment and administering an effective amount of an MHC class II α1 domain polypeptide or a nucleic acid encoding an MHC class II α1 domain polypeptide to the subject. In some examples, the MHC class II α1 domain is covalently linked to an antigenic determinant or peptide (such as those discussed above).

In some embodiments, the subject has an inflammatory and/or autoimmune disease or disorder, including but not limited to, systemic lupus erythematosus, Sjogren's syndrome, rheumatoid arthritis, type I diabetes mellitus, Wegener's granulomatosis, inflammatory bowel disease, polymyositis, dermatomyositis, multiple endocrine failure, Schmidt's syndrome, autoimmune uveitis, celiac disease, Addison's disease, adrenalitis, Graves' disease, thyroiditis, Hashimoto's thyroiditis, autoimmune thyroid disease, pernicious anemia, gastric atrophy, chronic hepatitis, lupoid hepatitis, atherosclerosis, presenile dementia, demyelinating diseases, multiple sclerosis, subacute cutaneous lupus erythematosus, hypoparathyroidism, Dressler's syndrome, myasthenia gravis, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyl), and telangiectasia), adult onset diabetes mellitus (Type II diabetes), male and female autoimmune infertility, ankylosing spondylitis, ulcerative colitis, Crohn's disease, mixed connective tissue disease, polyarteritis nedosa, systemic necrotizing vasculitis, juvenile onset rheumatoid arthritis, glomerulonephritis, atopic dermatitis, atopic rhinitis. Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, glomerulonephritis, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and Non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, rubulavirus, and Evan's syndrome. Additional inflammatory diseases include osteoarthritis, inflammatory lung disease (including chronic obstructive pulmonary lung disease), periodontal disease, polymyalgia rheumatica, atherosclerosis, systemic sclerosis, allergic rhinitis, and skin disorders (including dermatomyositis and psoriasis) and the like. In other embodiments, the subject has a retinal disorder, such as a retinal degeneration, such as retinitis pigmentosa, cone-rod dystrophy, Leber congenital amaurosis, or a maculopathy (for example, age-related macular degeneration, Stargardt-like macular degeneration, vitelliform macular dystrophy (Best disease), Mal atti a Leventinese (Doyne's honeycomb retinal dystrophy), diabetic maculopathy, occult macular dystrophy, and cellophane maculopathy). In other examples, a retinal disorder includes a retinopathy, such as autoimmune retinopathy, diabetic retinopathy, or vascular retinopathy. In still further examples, a retinal disorder includes retinal detachment or glaucoma. Retinal disorders may be progressive (for example, retinal degeneration or glaucoma) or acute (for example, retinal detachment). In additional examples, the subject is a subject with uveitis or optic neuritis. In other embodiments, the subject has had a stroke (such as ischemic stroke or hemorrhagic stroke). In still further examples, the subject is a subject with substance addiction, for example, a subject with cognitive or neuropsychiatric impairment induced by substance addiction.

In some embodiments, a subject is administered an effective amount of a composition including an MHC class II α1 domain or a portion thereof (such as a portion of an α1 domain which is capable of binding CD74 or decreasing expression and/or activity of CD74). Pharmaceutical compositions that include one or more of the MHC class II α1 domains disclosed herein (such as 2, 3, 4, 5, or more MHC class II α1 domains) can be formulated with an appropriate solid or liquid carrier, depending upon the particular mode of administration chosen. The pharmaceutically acceptable carriers and excipients useful in this disclosure are conventional. See, e.g., Remington: The Science and Practice of Pharmacy, The University of the Sciences in Philadelphia, Editor, Lippincott. Williams, & Wilkins, Philadelphia, Pa., 21^{st} Edition (2005). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

The dosage form of the pharmaceutical composition will be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, starch, or magnesium stearate. Actual methods of preparing such dosage forms are known, or will be apparent, to those skilled in the art.

In some examples, the pharmaceutical composition may be administered by any means that achieve their intended purpose. Amounts and regimens for the administration of the selected MHC class II α1 domain polypeptides or portion thereof (or a nucleic acid encoding such polypeptides) will be determined by the attending clinician. Effective doses for therapeutic application will vary depending on the nature and severity of the condition to be treated, the particular MHC class II α1 domain or portion thereof selected, the age and condition of the patient, and other clinical factors. Typically, the dose range will be from about 0.1 μg/kg body weight to about 100 mg/kg body weight. Other suitable ranges include doses of from about 100 μg/kg to about 50 mg/kg body weight, about 500 μg/kg to about 10 mg/kg body weight, or about 1 mg/kg to about 5 mg/kg body weight. The dosing schedule may vary from once a week to daily depending on a number of clinical factors, such as the subject's sensitivity to the protein. Examples of dosing schedules are about 1 mg/kg administered once a month, bi-weekly, once a week, twice a week, three times a week or daily; a dose of about 2.5 mg/kg once a week, twice a week, three times a week or daily; a dose of about 5 mg/kg once a week, twice a week, three times a week or daily; a dose of about 10 mg/kg once a week, twice a week, three times a week or daily; or a dose of about 30 mg/kg once a week, twice a week, three times a week or daily.

The pharmaceutical compositions that include one or more of the disclosed MHC class II α1 domain molecules can be formulated in unit dosage form, suitable for individual administration of precise dosages. In one specific, non-limiting example, a unit dosage can contain from about 1 ng to about 5 g of MHC class II α1 domain (such as about 10 μg to 1 g or about 10 mg to 100 mg). The amount of active compound(s) administered will be dependent on the subject being treated, the severity of the affliction, and the manner of administration, and is best left to the judgment of the prescribing clinician. Within these bounds, the formulation to be administered will contain a quantity of the active component(s) in amounts effective to achieve the desired effect in the subject being treated.

The compounds of this disclosure can be administered to humans or other animals on whose tissues they are effective in various manners such as topically, orally, intravenously, intramuscularly, intraperitoneally, intranasally, intradermally, intrathecally, subcutaneously, intraocularly, via inhalation, or via suppository. In one example, the compounds are administered to the subject subcutaneously. In another example, the compounds are administered to the subject intravenously. The particular mode of administration and the dosage regimen will be selected by the attending clinician, taking into account the particulars of the case (e.g., the subject, the disease, the disease state involved, and whether the treatment is prophylactic). Treatment can involve monthly, bi-monthly, weekly, daily or multi-daily doses of compound(s) over a period of a few days to months, or even years.

In some embodiments, the disclosed MHC class II α1 domain molecules can be included in an inert matrix for topical application. In some examples, the formulation can be injected into the eye, for example for intravitreal injection. As one example of an inert matrix, liposomes may be prepared from dipalmitoyl phosphatidylcholine (DPPC), such as egg phosphatidylcholine (PC). Liposomes, including cationic and anionic liposomes, can be made using standard procedures as known to one skilled in the art. Liposomes including one or more MHC class II α1 domains can be applied topically, either in the form of drops or as an aqueous based cream, or can be injected intraocularly. In a formulation for topical application, the MHC class II α1 domain is slowly released over time as the liposome capsule degrades due to wear and tear from the eye surface. In a formulation for intraocular injection, the liposome capsule degrades due to cellular digestion. Both of these formulations provide advantages of a slow release drug delivery system, allowing the subject to be exposed to a substantially constant concentration of the MHC class II α1 domain over time. In one example, the MHC class II α1 domain can be dissolved in an organic solvent such as DMSO or alcohol as previously described and contain a polyanhydride, poly(glycolic) acid, poly(lactic) acid, or polycaprolactone polymer. The MHC class II α1 domains can be included in a delivery system that can be implanted at various sites in the eye, depending on the size, shape and formulation of the implant, and the type of transplant procedure. Suitable sites include but are not limited to the anterior chamber, anterior segment, posterior chamber, posterior segment, vitreous cavity, suprachoroidal space, subconjunctiva, episcleral, intracorneal, epicorneal and sclera.

In some examples, an effective amount (for example, a therapeutically effective amount) of a disclosed MHC class II α1 domain polypeptide can be the amount of an MHC class II α1 domain polypeptide or an MHC class II α1 domain polypeptide including an antigen (such as a myelin protein antigen, a retinal antigen, or other antigen, such as those discussed above) necessary to treat or inhibit a disorder (such as an inflammatory and/or autoimmune disorder) in a subject. In other examples, a therapeutically effective amount of a disclosed MHC class II α1 domain polypeptide can be the amount of an MHC class II α1 domain polypeptide or an MHC class II α1 domain polypeptide including an antigen necessary to treat or inhibit a retinal disorder, stroke, or disorders associated with substance addiction (such as cognitive or neuropsychiatric impairment resulting from substance addiction).

The present disclosure also includes combinations of one or more of the disclosed MHC class II α1 domains with one or more other agents useful in the treatment of a disorder. In some examples, the compounds of this disclosure can be administered with effective doses of one or more therapies for inflammatory or autoimmune disorders, including but not limited to non-steroidal anti-inflammatory drugs, corticosteroids, methotrexate, anti-TNF compounds, mycopheonlate, aminoslicylates, antibiotics, interferons, glatiramer acetate, antibody therapies (such as rituximab or milatuzumab), or immunosuppressant or immunomodulator compounds. In another example, the compounds of this disclosure can be administered in combination with effective doses of one or more therapies for retinal disorders, including but not limited to, gene therapy, vitamin or mineral supplements (such as vitamins A, C, and/or E, or zinc and/or copper), anti-angiogenic therapy (such as ranibizumab or bevacizumab), photocoagulation, photodynamic therapy, lutein or zeaxanthin, corticosteroids, or immunosuppressants. Appropriate combination therapy for a particular disease can be selected by one of ordinary skill in the art. The term “administration in combination” or “co-administration” refers to both concurrent and sequential administration of the active agents.

### VI. Methods of Evaluating or Optimizing Treatment

Disclosed herein are methods for evaluating or optimizing efficacy of treatment of a disease or disorder (including but not limited to an inflammatory or autoimmune disorder) in a subject. The methods include determining CD74 expression or activity levels and determining efficacy of treatment or adjusting treatment (for example increasing or decreasing a dosage) with a polypeptide including an MHC class II α1 domain (or portion thereof), an MHC class II β1 domain, or a combination thereof based on the CD74 expression or activity level. In some examples, CD74 expression is increased in a subject with an inflammatory or autoimmune disorder (for example, as compared to a subject without the inflammatory or autoimmune disorder).

In some embodiments, the methods include determining efficacy of treatment of a disorder (such as those discussed in Section V, above) in a subject treated with a polypeptide including an MHC class II α1 domain (or portion thereof), an MHC class II β1 domain, or a combination thereof (such as aβ1α1 polypeptide). The methods include determining a CD74 expression or activity level in a sample from the subject. The CD74 expression or activity level is compared to a control and efficacy of treatment is determined. In some examples, the treatment is considered to be effective if the CD74 expression or activity level is less than or equal to the control. In other examples, the treatment is considered to be suboptimally effective or not effective if the CD74 expression or activity level is greater than the control.

In other embodiments, the methods include optimizing efficacy of treatment of a disorder (such as those discussed in Section V, above) in a subject. The methods include administering a polypeptide including an MHC class II α1 domain (or portion thereof), an MHC class II β1 domain, or a combination thereof (such as a polypeptide) to a subject with the disorder and determining a CD74 expression or activity level in a sample from the subject. The CD74 expression or activity level is compared to a control and a dosage of the polypeptide to be subsequently administered to the subject is determined. In some examples, the dosage of the polypeptide can be increased if the CD74 expression of activity level is greater than the control. In other examples, the dosage of the polypeptide can be maintained or decreased if the CD74 expression or activity level is less than or equal to the control.

In further embodiments, the methods include treating or inhibiting a disorder in a subject by administering a polypeptide including an MHC class II α1 domain (or portion thereof), an MHC class II β1 domain, or a combination thereof (such as a β1α1 polypeptide) to a subject with the disorder and increasing the dosage of the polypeptide if CD74 expression or activity level in a sample from the subject is greater than a control or maintaining or decreasing the dosage of the polypeptide if CD74 expression or activity level in a sample from the subject is less than or equal to a control.

In some examples, a level of CD74 expression or activity that is greater than a control (such as statistically significantly greater than a control) indicates a need to increase the amount or dosage of the MHC class II polypeptide (such as an MHC class II α1 domain polypeptide or portion thereof or an MHC class II β1α1 polypeptide) subsequently administered to the subject. In other examples, a level of CD74 expression or activity that is less than a control (such as statistically significantly less than a control) indicates a need to maintain or decrease the amount or dosage of the MHC class II polypeptide (such as an MHC class II α1 domain polypeptide or portion thereof or an MHC class II β1α1 polypeptide) subsequently administered to the subject. In some examples, the dosage of the MHC class II polypeptide (such as an MHC class II α1 domain polypeptide or an MHC class II β1α1 polypeptide) is adjusted to produce a CD74 expression or activity level that is about equal to that of a control. One of ordinary skill in the art can adjust dosage (for example, increase or decrease dosage) based on the determined CD74 expression or activity, the disease or disorder, the condition of the subject, and other factors. In some examples, the dosage is adjusted and CD74 expression or activity level is determined after administration of the adjusted dose (for example, about 1, 2, 3, 4, 5, 6, 7, 10, 14, or more days after administration of the adjusted dose). The dosage can be maintained or adjusted again, based on the level of CD74 expression or activity. This can be repeated as many times as necessary to achieve the desired level of CD74 expression or activity, as well as the desired inhibition of the disease or disorder (for example amelioration of symptoms of the disease or disorder).

The control is any suitable control for CD74 expression or activity level. In some embodiments, the control is a sample obtained from a healthy subject or population of healthy subjects. In other embodiments, the control is a historical control or standard reference value or range of values (such as a previously tested control sample, such as a group of samples that represent baseline or normal values, such as the level of CD74 expression or activity in healthy subjects). In some examples, the control is CD74 expression level, such as CD74 RNA (for example CD74 mRNA) or CD74 protein level or amount in a sample from a healthy subject or a reference value for CD74 RNA or protein level or amount in a population of healthy subjects (such as an average CD74 level in a population of healthy subjects). In other examples, the control is CD74 activity level, such as CD74 activity in a sample from a healthy subject or a reference value for CD74 activity in a population of healthy subjects (such as an average CD74 activity level in a population of healthy subjects). In further examples, the control is CD74 expression or activity level in a sample from an untreated subject or a subject prior to beginning treatment with an MHC class II β1α1 polypeptide or an MHC class II ad domain polypeptide (such as the same subject or a different subject with the same disorder). In other examples, a control is CD74 expression or activity level in a cohort of healthy subjects (for example, a cohort of subjects matched for one or more of age, gender, disorder, or other clinical factors).

In some embodiments, the sample is from a subject who has been administered an MHC class II polypeptide including an MHC α1 domain, an MHC class II β1 domain, or a combination thereof or to whom such an MHC class II polypeptide is being administered. In some examples, the polypeptide includes an MHC class II α1 domain and does not include an MHC class II α2, β1, or β2 domain (for example, an MHC class II α1 domain polypeptide disclosed herein). In other examples, the polypeptide includes an MHC class II β1 domain and an MHC class II α1 domain in which the amino terminus of the α1 domain is covalently linked (for example directly or by a peptide linker) to the carboxy terminus of the β1 domain (a β1α1 polypeptide). MHC class II β1α1 polypeptides have been previously described. See, e.g., U.S. Pat. No. 6,270,772; U.S. Pat. App. Publ. Nos. 2005/0142142, 2009/0280135, 2011/0262479, 2011/0008382, 2011/0217308, each of which are incorporated herein by reference in their entirety. In some examples, the MHC class II α1 domain polypeptide or the MHC class II β1α1 polypeptide also include an antigenic determinant, for example covalently or non-covalently linked to the MHC class II polypeptide.

Determining CD74 Expression or Activity Level

Methods of determining CD74 expression or activity level are known to one of ordinary skill in the art. In some examples, determining CD74 expression includes determining expression (such as presence, absence, or an amount) of a CD74 nucleic acid or protein. In some examples, the methods include determining the presence or amount of one, two, three, or more isoforms of CD74 (such as alternatively spliced isoforms). In other examples, determining CD74 activity includes determining CD74 protein activity, for example a cellular or molecular event mediated by or modified by CD74 activity.

Samples suitable for determining CD74 expression or activity level include a biological specimen containing nucleic acid (e.g., DNA or RNA (including mRNA)), protein, or combinations thereof, obtained from a subject. Examples include, but are not limited to, peripheral blood, fine needle aspirate, urine, saliva, tissue biopsy, surgical specimen, and autopsy material. In one example, a sample includes a blood sample (such as blood; derivatives and fractions of blood, such as serum) or isolated or purified cell populations (for example, T cells, B cells, PBMC, lymphocytes, and so on, including partially isolated or partially purified cell populations). In some examples, the sample includes, consists essentially of, or consists of monocytes, B cells, CD11b⁺ cells, CD34⁺ cells, CD4⁺ cells, CD19⁺ cells, CD74⁺ cells, or a combination of two or more thereof.

CD74 Expression

Gene expression can be evaluated by detecting RNA (such as mRNA) encoding CD74. RNA can be isolated from a sample from a subject (such as a blood sample) using methods well known to one skilled in the art, including commercially available kits. General methods for RNA extraction are well known in the art and are disclosed in standard textbooks of molecular biology, including Ausubel et al. Current Protocols of Molecular Biology, John Wiley and Sons (1997). Methods for RNA extraction from paraffin embedded tissues are disclosed, for example, in Rupp and Locker, Biotechniques 6:56-60 (1988), and De Andres et al., Biotechniques 18:42-44 (1995).

Methods of determining gene expression include methods based on hybridization analysis of polynucleotides, methods based on sequencing of polynucleotides, and proteomics-based methods. One of ordinary skill in the art can obtain suitable primers and/or probes for use in methods for determining CD74 gene expression. In some examples, mRNA expression in a sample is quantified using Northern blotting or in situ hybridization (Parker & Barnes, Methods in Molecular Biology 106:247-283, 1999); RNAse protection assays (Hod, Biotechniques 13:852-4, 1992); and PCR-based methods, such as reverse transcription polymerase chain reaction (RT-PCR) (Weis et al., Trends in Genetics 8:263-4, 1992), quantitative RT-PCT, or TaqMan RT-PCR. Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). In one example, RT-PCR can be used to compare mRNA levels in different samples, for example in samples from a subject and a control, to determine CD74 expression.

To minimize errors and the effect of sample-to-sample variation, RT-PCR can be performed using an internal standard. The ideal internal standard is expressed at a constant level among different tissues, and is unaffected by an experimental treatment. RNAs commonly used to normalize patterns of gene expression are mRNAs for the housekeeping genes GAPDH, β-actin, and 18S ribosomal RNA.

In situ hybridization (ISH) is another method for detecting and comparing expression of genes of interest. ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to assay expression patterns in a tissue, such as the expression of cancer survival factor-associated genes.

In some embodiments of the detection methods, the expression of one or more “housekeeping” genes or “internal controls” can also be evaluated. These terms include any constitutively or globally expressed gene (or protein, as discussed below) whose presence enables an assessment of CD74 gene (or protein) levels. Such an assessment includes a determination of the overall constitutive level of gene transcription and a control for variations in RNA (or protein) recovery.

In some examples, expression of CD74 protein is analyzed. Antibodies specific for CD74 can be used for detection and quantitation of protein expression by one of a number of immunoassay methods that are well known in the art, such as those presented in Harlow and Lane (Antibodies, A Laboratory Manual, CSHL, New York, 1988). Methods of constructing such antibodies are known in the art. In addition, such antibodies may be commercially available. Exemplary commercially available antibodies include catalog numbers sc-70781, sc-166047, sc-81626, and sc-65272 from Santa Cruz Biotechnology (Santa Cruz, Calif.); catalog numbers ab9514, ab22603, ab64772, and ab108402 from Abcam (Cambridge, Mass.), and catalog numbers MAB35901 and AF3590 from R&D Systems (Minneapolis, Minn.).

Any standard immunoassay format (such as ELISA, Western blot, flow cytometry, or RIA assay) can be used to measure protein levels. Thus, in one example, polypeptide levels of CD74 protein in a sample can readily be evaluated using these methods. Immunohistochemical techniques can also be utilized for CD74 detection and quantification. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

CD74 Activity

CD74 (invariant chain, Ii) is a type II transmembrane glycoprotein containing a trimerization domain flanked by two highly unstructured regions (Jasanoff et al., Immunity 10:761-768, 1999). One role of CD74 is to chaperone newly synthesized MHC class II through the endocytic pathway to the cell surface of APC (Cresswell, Cell 84:505-507, 1996). CD74, in combination with CD44, CXCR2, and CXCR4, is also the receptor for macrophage migration inhibitory factor (MIF) (Leng et al., J. Exp. Med. 197:1467-1479, 2003; Naujokas et al., Cell 74:257-268, 1993). MIF signal transduction through CD74 includes activation of extracellular signal-regulated kinase (ERK) 1/2, NF-κB activation, Bcl-2 expression, and IL-8 secretion (e.g., Leng et al., J. Exp. Med. 197:1467-1476, 2003; Starlets et al., Blood 107:4807-4816, 2006; Binsky et al., Proc. Natl. Acad. Sci. USA 104:13408-13413, 2007; Gore et al., J. Biol. Chem. 283:2784-2792, 2008). One result of activation of signaling through MIF-activated CD74 is an increase in cell proliferation or survival, or a decrease in apoptosis.

One of ordinary skill in the art can identify and determine levels of CD74 activity. In some examples. CD74 activity is determined indirectly, for example by determining activity of one or more downstream effectors of CD74 or one or more cell phenotypes regulated by CD74 (such as cell proliferation, survival, or migration). In other examples, CD74 activity is determined by MIF binding or binding of an MHC class II polypeptide, such as an MHC class II β1α1 polypeptide or MHC class II α1 polypeptide, for example, as described in Example 3, below.

In some embodiments, CD74 activity is determined in a sample from a subject (such as a subject with an inflammatory and/or autoimmune disease or a control), for example a sample including monocytes, B cells, CD11b⁺ cells, CD34⁺ cells, CD4⁺ cells, CD19⁺ cells, CD74⁺ cells, or a combination of two or more thereof. In some examples, CD74 activity is determined by contacting the sample with MIF and measuring activity of a downstream effector of CD74, such as ERK1/2 activity (for example, phosphorylation), NF-κB activation (for example, transcription mediated by pp65/RelA), Bcl-2 expression. ICAM-1 expression, and/or IL-8 secretion. In some examples, contacting the sample with MIF results in an increase in activity of at least one downstream effector of CD74. This CD74 activity level can be compared with a control which has been contacted with MIF, such as a sample from a healthy subject or an untreated subject with the disorder. Methods for determining these activities (for example, utilizing Western blotting, flow cytometry, ELISA, reporter assays, or PCR-based methods) are well known to one of ordinary skill in the art. Exemplary assays are shown in Leng et al., J. Exp. Med. 197:1467-1476, 2003; Starlets et al., Blood 107:4807-4816, 2006; Binsky et al., Proc. Natl. Acad. Sci. USA 104:13408-13413, 2007; and Gore et al., J. Biol. Chem. 283:2784-2792, 2008; each of which is incorporated herein by reference in their entirety.

In other examples, CD74 activity is determined by contacting the sample with MIF and determining a cell phenotype, such as cell proliferation, cell migration, and/or apoptosis after a sufficient period of time to observe the phenotype. Methods of measuring cell proliferation are well known in the art. For example, incorporation of a DNA label (for example 5-bromo-2-deoxyuridine (BrdU), [³H]thymidine, or a fluorescent label), MTT or XTT assay, or cell counting. Methods of measuring apoptosis are well known in the art. For example, apoptotic cell death can be characterized by cell shrinkage, membrane blebbing and chromatin condensation culminating in cell fragmentation. Cells undergoing apoptosis also display a characteristic pattern of internucleosomal DNA cleavage. Methods of measuring cell migration, such as a Boyden chamber assay, are well known to one of ordinary skill in the art. The CD74 activity level can be compared with a control which has been contacted with MIF, such as a sample from a healthy subject or an untreated subject with the disorder. Exemplary assays are shown in Leng et al., J. Exp. Med. 197:1467-1476, 2003; Starlets et al., Blood 107:4807-4816, 2006; Binsky et al., Proc. Natl. Acad. Sci. USA 104:13408-13413, 2007; and Gore et al., J. Biol. Chem. 283:2784-2792, 2008; each of which is incorporated herein by reference in their entirety.

The following examples are provided to illustrate certain particular features and/or embodiments. These examples should not be construed to limit the disclosure to the particular features or embodiments described.

### EXAMPLES

### Example 1

### Materials and Methods

Mice:

DR*1501-Tg, DR*1502-Tg and MBP-TCR/DR2-Tg mice were bred in-house at the Veterinary Medical Unit, Portland Veterans Affairs Medical Center and used at 8-12 weeks of age. All procedures were approved and performed according to institutional guidelines.

Induction of EAE in DR2-Tg and MBP-TCR/DR2-Tg Mice:

HLA-DR2 mice were screened by FACS for the expression of the HLA transgenes (McMahan et al, J Biol Chem 278:30861-30970, 2003). HLA-DR2 positive male and female mice between 8 and 12 weeks of age were immunized subcutaneously at four sites on the flanks with 0.2 ml of an emulsion of 200 μg immunogenic peptide and complete Freund's adjuvant containing 400 μg of heat-killed Mycobacterium tuberculosis H37RA (Difco, Detroit, Mich.) (Vandenbark et al, J Immunol 171:127-133, 2003; Buenafe et al, Immunology 130:114-124, 2010). In addition, mice were given pertussis toxin (Ptx) from List Biological Laboratories (Campbell, Calif.) on days 0 and 2 post-immunization (75 ng and 200 ng per mouse, respectively). Immunized mice were assessed daily for clinical signs of EAE on a 6 point scale of combined hind limb and forelimb paralysis scores. For hind limb scores: 0=normal; 0.5=limp tail or mild hind limb weakness (e.g., a mouse cannot resist inversion after a 90° turn of the base of the tail); 1=limp tail and mild hind limb weakness; 2=limp tail and moderate hind limb weakness (e.g., an inability of the mouse to rapidly right itself after inversion); 3=limp tail and moderately severe hind limb weakness (e.g., inability of the mouse to right itself after inversion and clear tilting of hind quarters to either side while walking); 4=limp tail and severe hind limb weakness (hind feet can move but drag more frequently than face forward); 5=limp tail and paraplegia (no movement of hind limbs). Front limb paralysis scores are either 0.5 for clear restriction in normal movement or 1 for complete forelimb paralysis. The combined score is the sum of the hind limb score and the forelimb score.

Rarely, there was mortality of HLA-DR2 mice with severe EAE and in these cases, mice were scored as a 6 for the remainder of the experiment. Mean EAE scores and standard deviations for mice grouped according to initiation of RTL or vehicle treatment were calculated for each day and summed for the entire experiment (Cumulative Disease Index, CDI, represents total disease load). Daily mean scores were analyzed by a two-tailed Mann Whitney U test for nonparametric comparisons between vehicle and pDR2 treatment groups. Mean CDIs were analyzed by a one way ANOVA with Tukey post-test, and a nonparametric one way Kruskal-Wallis ANOVA with Dunn's multiple comparisons post-test to confirm significance between all groups.

pMHC Treatment of EAE in DR2-Tg Mice:

pDR2 constructs (Table 3) were injected subcutaneously daily for 5 days at the indicated doses to treat EAE induced in HLA-DR2-Tg and MBP-TCR/DR2-Tg mice and clinical signs were scored as described above. For neutralization experiments, DR*1501-Tg mice were treated subcutaneously with vehicle (20 mM Tris-HCl pH 8.0 with 5% w/v D-glucose), 20 μg RTL342M (pDR2/mMOG-35-55), or 20 μg RTL342M pre-incubated at a 1:1 (40 μg) or 1:2 (80 μg) molar ratio with Fab1B11 (specific for two-domain DR2 constructs) or FabD2 (specific for pDR4/GAD-555-567 constructs). Fab1B11 alone in vehicle was run as a negative control. Methods were previously described in Dahan et al., Eur. Immunol. 41:1465-1479, 2011.

TABLE 3
pDR2 constructs

| pDR2 Construct | Description |
|---|---|
| RTL302-5D | Human HLA DR2 (DRB1*1501/DRA*0101) β1α1 |
| | domains (V102D, I104D, A106D, F108D, L110D) |
| RTL340 | RTL302-5D/MBP-85-99 |
| RTL342M | RTL302-5D/mMOG-35-55 |
| RTL1000 | RTL302-5D/hMOG-35-55 |

Flow Cytometry:

Analysis of naive DR2 PBMC subtypes was preformed using four-color (fluorescein isothiocyanate (FITC), phycoerythrin (PE), propidium iodide, allophycocyanin) flow cytometry. Blood was collected from naive DR2 mice through cardiac exsanguination into 1X PBS/EDTA. After washing blood in 1X PBS, the red blood cells were lysed with 1X RBC lysis buffer (eBioscience, Inc., San Diego, Calif.) followed by two washes in RPMI. One million cells were incubated in RPMI with 1 μg unlabeled pDR2, 1 μg pDR2FITC, or 1 μg Fab Neutralized pDR2FITC for one hour at 37° C. For samples assessing Fab neutralization of pDR2 binding, pDR2s were incubated with 1:1 molar ratio of Fab1B11 or FabD2 for two hours at room temperature prior to incubation with cells, pDR2 incubation was followed directly by a 30 minute incubation at 4° C. with CD3 PE (ebioscience, Inc., San Diego, Calif.), CD74 PE (Santa Cruz Biotechnology, Inc., Santa Cruz, Calif.), or CD11b PE, CD11b allophycocyanin, CD11c allophycocyanin, CD19 allophycocyanin (BD Pharmingen, San Diego, Calif.). Antibodies and pDR2 were thoroughly removed by two additional washes in 1X PBS/0.5% BSA staining media. Cells were resuspended in staining media containing propidium iodide and immediately analyzed with a FACSCALIBUR™ using FCS EXPRESS™ software (BD Biosciences, San Jose, Calif.). Data represent 100,000 gated live monocytes and lymphocytes (in vivo analyses) or 10,000 gated live monocytes (in vitro analyses).

Microscopy and Imaging of pDR2 Binding:

CD11b+ cells were negatively isolated from DR*1501/GFP-Tg mice by Mouse Monocyte Enrichment Kit (Stem Cell Technologies, Vancouver, Canada) and treated with 10 mg/ml RTL342M tagged with Alexa Fluor® 546 in RPMI for 40 minutes. The images were acquired on a high resolution wide field Core DV microscopy system (Applied Precision, Issaquah, Wash.) utilizing an Olympus IX71 inverted microscope with a proprietary XYZ stage enclosed in a controlled environment chamber: differential interference contrast (DIC) transmitted light and a solid state module for fluorescence. A Coolsnap ES2 HQ camera was used to acquire images as optical axis with a 60× (numerical aperture, 1.42) Plan Apo N objective in 2 colors, FITC and TRITC. The pixel size was 0.10704 microns. The images were deconvolved with the appropriate OTF (optical transfer function) using an iterative algorithm of 10 iterations. Histograms were optimized for the most positive image and applied to all the other images for consistency before saving the images as 24 bit merged TIFF. Data were visualized and analyzed using Imaris® (Bitplane), and MATLAB® (Mathworks, Natick, Mass.).

Protein Purification and Labeling:

Cloning, expression and purification of the pDR2 proteins from inclusion bodies produced in Escherichia coli was as described in Chang et al., J. Biol. Chem. 276:24170-24176, 2001.

Biotinylation of Cell Surface Proteins and Cell Lysis:

Splenocytes from DR*1501-Tg or MHC Class II-knockout mice were collected in RPMI and kept on ice before being used in experiments. Cells were washed extensively with cold PBS at pH 8.0 and biotinylated with EZ-Link® Sulfo-NHS-LC-Biotin (Pierce Biotechnology, Cat No. 21335) on ice for 15 minutes to prevent over-labeling. The reaction was quenched by diluting the biotinylated cell suspension 5X with TEN (50 mM Tris, 2 mM EDTA, 150 mM NaCl) buffer at pH 7.4. Subsequent washes were carried out with TEN buffer to remove the biotinylation reagent and the pellet was kept frozen until lysis. Cell pellet was thawed on ice and lysis was performed in TEN buffer containing 1% of Triton X-100 (TEN-TX100) or 1% CHAPS (TEN-CHAPS) for 30 to 60 minutes in the presence of protease inhibitors (Halt Protease Inhibitor Cocktail, Pierce Biotechnology) in addition to 1 μM PMSF (Sigma-Aldrich, St. Louis, Mo.). After lysis, cells were sedimented at 14,000 RPM for 15 minutes at 4° C. and the supernatant was collected for further analysis.

Direct and Competitive Binding Assays:

Two million splenocytes from DR*1501-Tg mice were incubated with increasing concentrations of AlexaFluor® 488-labeled pDR2/hMOG-35-55 (RTL1000) in RPMI for one hour in ice (to minimize internalization through phagocytic mechanisms). The cells were then lysed in 100 μl of 6 M urea to dissociate and solubilize bound labeled ligand for subsequent analysis. The lysate was centrifuged to remove insoluble material (nuclei, organelles) and then the supernatant was separated SDS-PAGE electrophoresis in a 10-20% polyacrylamide gel. The gel was then scanned for labeled pDR2. Band fluorescence intensity was quantified by densitometry using Quantity One® software (BioRad) and plotted vs. the labeled RTL1000 concentration. Data were analyzed using GraphPad Prism® Software and fitted to 1- or 2-binding site mathematical models.

Competitive binding was carried out using 2 million splenocytes and a constant concentration of labeled RTL1000 (280 nM) in the presence of increasing concentrations of unlabeled pDR2 constructs (0-8 μM). Afterwards, cells were washed, lysed, and analyzed as described above for saturation assays in whole cells. Results are presented as EC50, the concentration of competitor required to compete for half of the specific binding.

Immunoprecipitation and Direct and Competitive Binding Assays for CD74 and MHC Class II Molecules:

For immunoprecipitation experiments, antibody TU39 (BD Pharmingen) was pre-bound to bead-conjugated Protein A for 2 hours in ice-cold TEN-TX100 or TEN-CHAPS followed by RTL binding to these complexes. Lysate (previously pre-cleared with bead-conjugated Protein A) was added to the tube and binding was carried out overnight at 4° C. with soft orbital shaking Samples were extensively washed with TEN buffer and the appropriate detergent. For CD74 immunoprecipitation, ln-1 mAb was adsorbed to bead-conjugated Protein L as described above and pre-cleared lysate was added to this mixture. When necessary, bound material was eluted by boiling the immune complexes in electrophoresis sample buffer and analyzed by SDS-PAGE and Western blot.

For direct binding of RTL constructs, Protein-L/ln-1/CD74 complexes were prepared as described above. Labeled ligand constructs were incubated for 4 hours at 4° C. in 1% CHAPS in TEN buffer and free ligand was removed by extensive washing. Elution of bound proteins was carried out as described above and the eluate was analyzed by SDS-P AGE. Competition experiments were carried out using 2:1 molar ratio of labeled RTL1000 to “cold” competitor in a 500 μl reaction volume at 4° C. for 3 to 4 hours in TEN-CHAPS buffer. A competition between RTL1000 and DR-α1 domain for the binding to CD74 was done by using 0.160 nmol (4 μg) of labeled RTL1000 and increasing concentrations (0, 0.032, 0.096, 0.320, and 0.960 nmol) of the DR-α1 domain in a total reaction volume of 0.5 ml. Under these conditions the final concentration of RTL1000 was 320 nM. In all assays involving fluorescent labeling, the chromophore was detected by scanning at the appropriate wavelength using a Molecular Imager® FX scanner (Bio-Rad) and the fluorescence intensity was determined using Quantity One® software (Bio-Rad) associated with the imager.

In order to test ligand binding to the separate components of the RTL receptor, DR*1501-Tg splenocytes were biotinylated and lysed as described above and CD74 was immunoprecipitated overnight at 4° C. with the anti-CD74 monoclonal antibody ln-1. Immune complexes (Protein L-beads/ln-1/CD74) were analyzed for their ability to bind FITC-labeled pDR2RTL constructs by performing a direct binding saturation with increasing concentration of the different constructs (0 to 10 nM). Binding was carried out for 4 hours at 4° C. with gentle shaking and in the presence of 1% CHAPS in TEN buffer. Afterwards the complexes were washed thoroughly with 1% CHAPS/TEN and once with only TEN buffer to remove excess detergent. Bound proteins were eluted in 2% SDS electrophoresis sample buffer at 90° C. for 6 to 8 minutes and beads were sedimented. After the supernatant was collected and proteins separated using 10-20% SDS-PAGE, the gel was scanned for FITC chromophore and quantified by densitometry. Ligand concentration was plotted versus the fluorescence intensity of the bands and the curve generated was fit to one or two binding sites with the GraphPad Prism® Software. Likewise, MHC class II from DR*1501-Tg mice was purified with L243 monoclonal antibody conjugated to Protein-L beads using the CD74-depleted lysate. Under these conditions a homogeneous preparation of MHC class II from DR*1501-Tg mice was isolated. Direct binding to saturating concentrations of the pDR2 constructs was carried out and analyzed as described above.

Electrophoresis, Western Blotting and LC-MS/MS:

After elution from immunoprecipitates, proteins were separated using 10-20% SDS-PAGE and visualized by Coomassie Blue staining, or they were blotted to PVDF and detected with streptavidin-conjugated PE. Relevant proteins detected by PE staining were localized in a replica gel stained with Coomassie Blue, the gel bands were cut, digested with trypsin and characterized by LC-MS/MS.

Surface Plasmon Resonance:

Histone complex (Sigma) was coupled to a CM5 biosensor chip by standard amine coupling in 10 mM NaOAc, pH 6.0, with a resulting final resonance of 6597 resonance units (RUs). Ethanolamine was coupled in a separate flow cell as a negative control. Kinetic measurements were performed on a Biacore 3000 in HBS-EP buffer (10 mM HEPES, pH 7.4, 150 mM NaCl, 3 mM EDTA, 0.005% P20) at a flow rate of 20 μl/min with no evidence of mass transfer effects. pDR2/hMOG-35-55 was injected in a series of concentrations and the surfaces regenerated with 50 mM NaOH before each injection. Fitting of kinetic data to a 1:1 binding model with baseline drift was performed using Biaevaluation 3.0 software (GE Healthcare, Piscataway, N.J.).

RTL342M (pDR2/mMOG-35-55) Inhibition of MIF-Enhanced Expression of ICAM-1:

Splenocytes were isolated from DR*1501-Tg mice and cultured in the presence or absence of 10 μg/ml RTL342M in complete RPMI medium 1640 with 2% heat inactivated FCS at 37° C. in 5% CO₂ for 1 hour. Cells were stimulated with 10 ng/ml LPS (E. coli, serotype 055:B5, Sigma-Aldrich) and 100 ng/ml recombinant MIF (R&D systems, Minneapolis, Minn., USA) for 1 hour before harvesting. Total RNA was isolated from splenocytes using an RNeasy® cultured cell kit according to the manufacturer's instructions, which included a DNase step (Qiagen, Valencia, Calif., USA). Quantitative real time PCR was performed using the ABI 7000 sequence detection system with gene-on-demand assay products (Applied Biosystems) for: ICAM-1 (Assay ID: Mm00516023_m1). GADPH housekeeping gene was amplified as an endogenous control. Primers were used according to manufacturer's instructions.

### Example 2

### Treatment of EAE in DR2-Tg Mice with pMHC Constructs

This example describes the effect of pMHC constructs and peptides on EAE in DR2 transgenic mice.

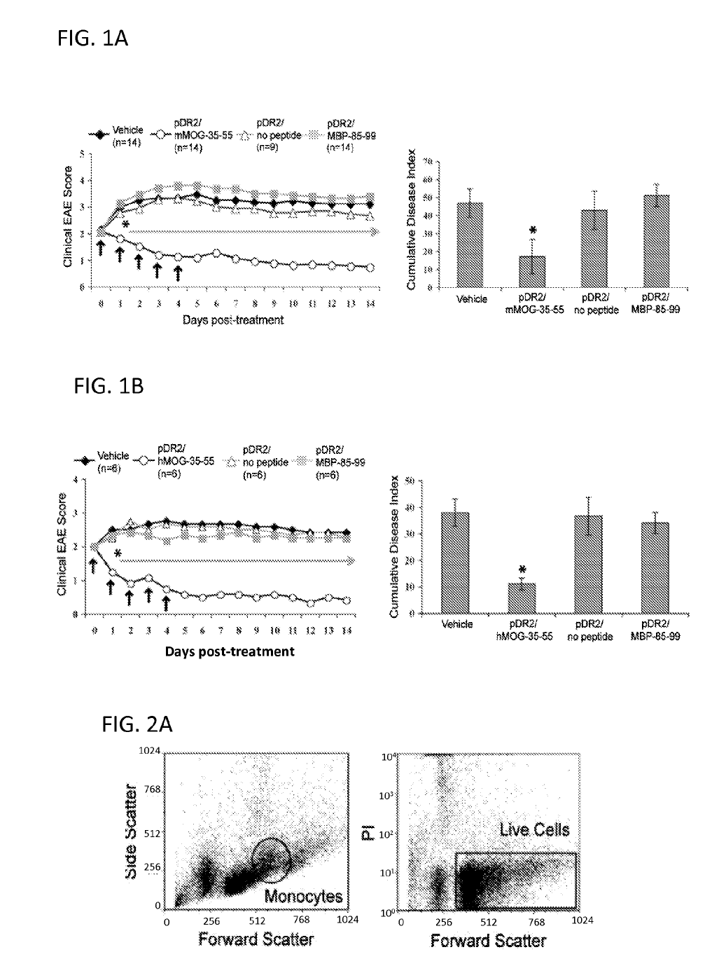

Specificity of pDR2 treatment of EAE was evaluated in two different strains of DR2-Tg mice, including DR*1501-Tg mice (DRA:DRβ1*1501 strain that develops EAE only after injection of mouse (m)MOG-35-55 peptide (Link et al., Clin. Immunol. 123:95-104, 2007)) and DR*1502-Tg mice (I-E^{b}:DRβ1*1502 strain that develops EAE after injection of human (h)MOG-35-55 peptide (Chou et al., J. Neurosci. Res. 77:670-680, 200)). DR*1501-Tg mice were treated effectively after disease onset with the cognate mMOG-35-55 peptide (SEQ ID NO: 50) tethered to pDR2 (FIG. 1A; Table 4). DR*1502-Tg mice were effectively treated with the cognate hMOG-35-55 peptide (SEQ ID NO: 51) tethered to the same pDR2 platform (FIG. 1B; Table 5), but not with pDR2/MBP-85-99 (SEQ ID NO: 56), containing a non-cognate encephalitogenic peptide (different than that used for disease induction) or pDR2 without a tethered peptide (pDR2/no peptide, FIGS. 1A and 1B; Tables 4 and 5).

TABLE 4
Summary of EAE progression in DR*1501-Tg mice
treated with the indicated constructs

| | Inci- | | | Mor- | |
|---|---|---|---|---|---|
| Group | dence | Onset | Peak | tality | CDI |
| Vehicle | 14/14 | 11.6 ± 1.0 | 3.5 ± 0.5 | 0 | 47.0 ± 7.9 |
| RTL342M | 14/14 | 11.3 ± 0.6 | 2.2 ± 0.3 | 0 | 17.1 ± 9.6* |
| RTL302-5D | 9/9 | 11.7 ± 1.1 | 3.3 ± 0.9 | 0 | 42.9 ± 10.7 |
| RTL340 | 14/14 | 11.0 ± 0.0 | 3.9 ± 0.5 | 0 | 51.2 ± 6.2 |

*p < 0.03 vs. vehicle, RTL340, and RTL302-5D

TABLE 5
Summary of EAE progression in DR*1502-Tg mice
treated with the indicated constructs

| | Inci- | | | Mor- | |
|---|---|---|---|---|---|
| Group | dence | Onset | Peak | tality | CDI |
| Vehicle | 6/6 | 14.5 ± 1.4 | 2.8 ± 0.3 | 0 | 38.0 ± 5.2 |
| RTL1000 | 6/6 | 14.7 ± 1.4 | 2.0 ± 0.0 | 0 | 11.1 ± 2.2* |
| RTL302-5D | 6/6 | 15.0 ± 1.7 | 2.9 ± 0.5 | 0 | 36.8 ± 7.1 |
| RTL340 | 6/6 | 15.0 ± 1.7 | 2.6 ± 0.2 | 0 | 34.2 ± 4.0 |

*p < 0.0023 vs. vehicle, RTL340, and RTL302-5D

### Example 3

### pDR2Binding Sites

This example describes identification of pDR2 binding sites on PBMC.

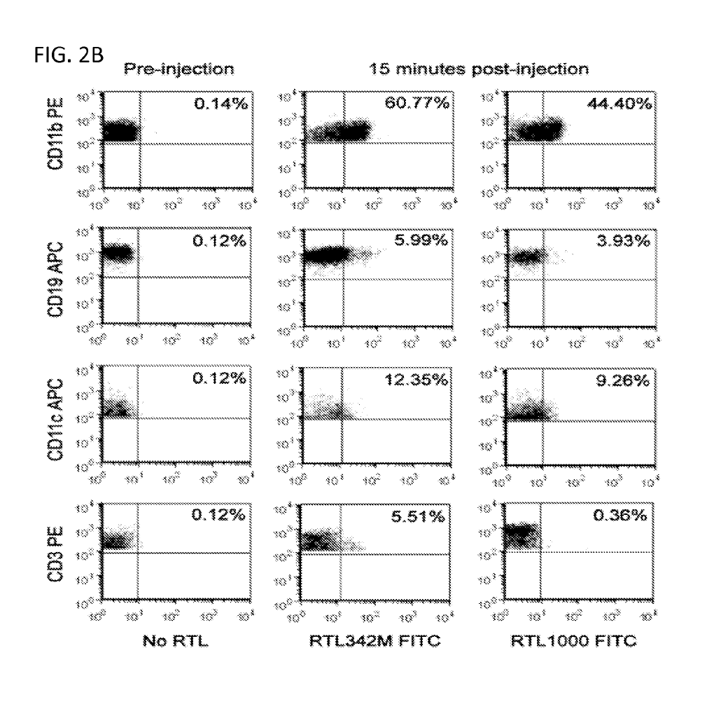

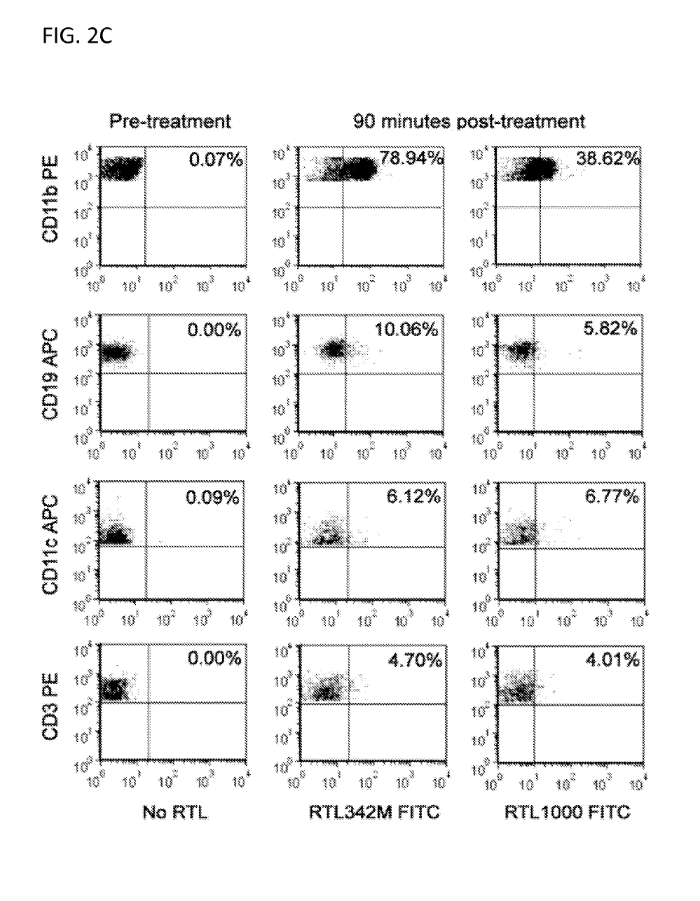

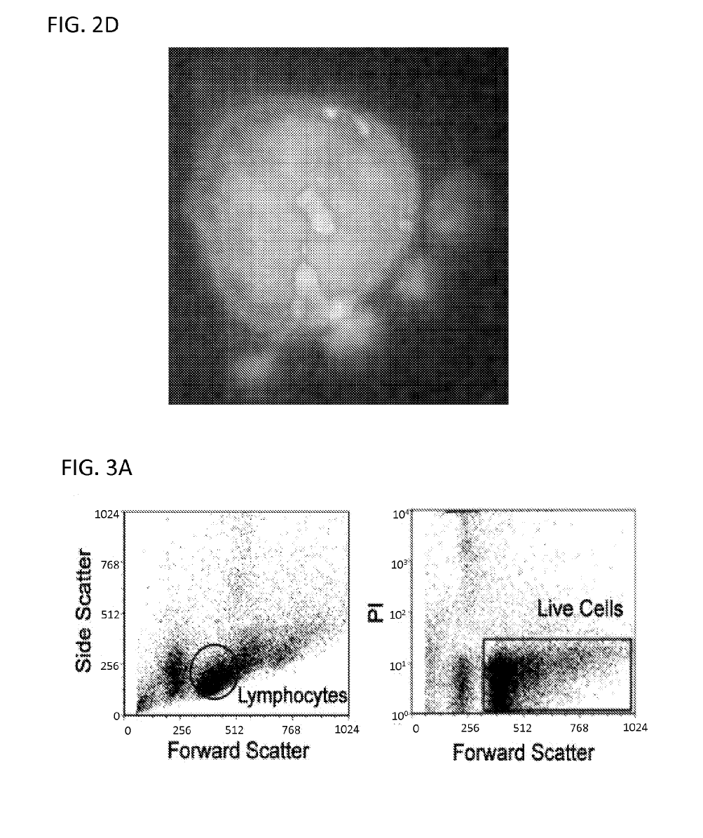

Naïve DR*1501-Tg mice were injected i.v. with pDR2 tethered to RTL342M-FITC or RTL1000-FITC. Viable PBMC subtypes were evaluated for pMHC binding in the monocyte gate using a flow cytometer. The monocyte population was identified using forward scatter and side scatter and live monocytes were identified using a combination of forward scatter and propidium iodide staining (FIG. 2A).

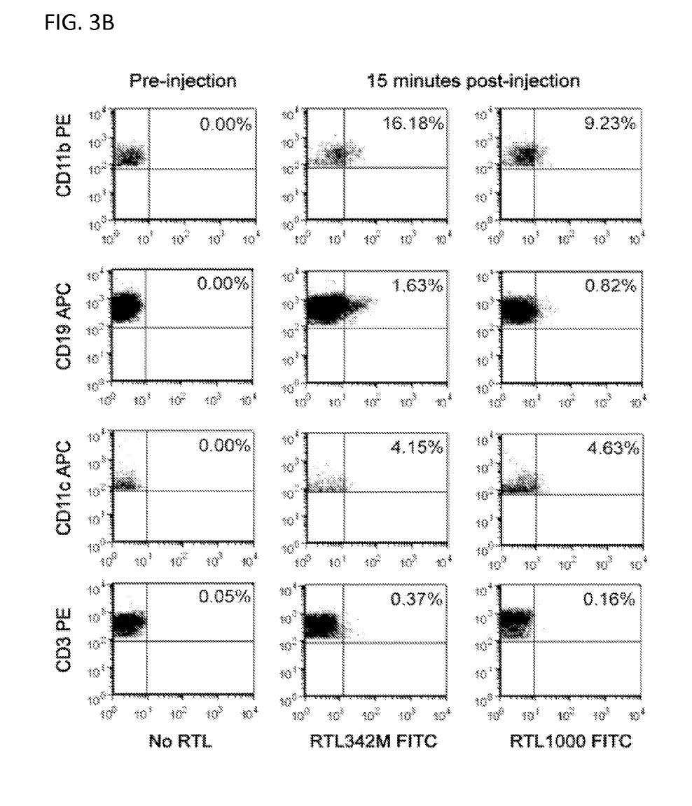

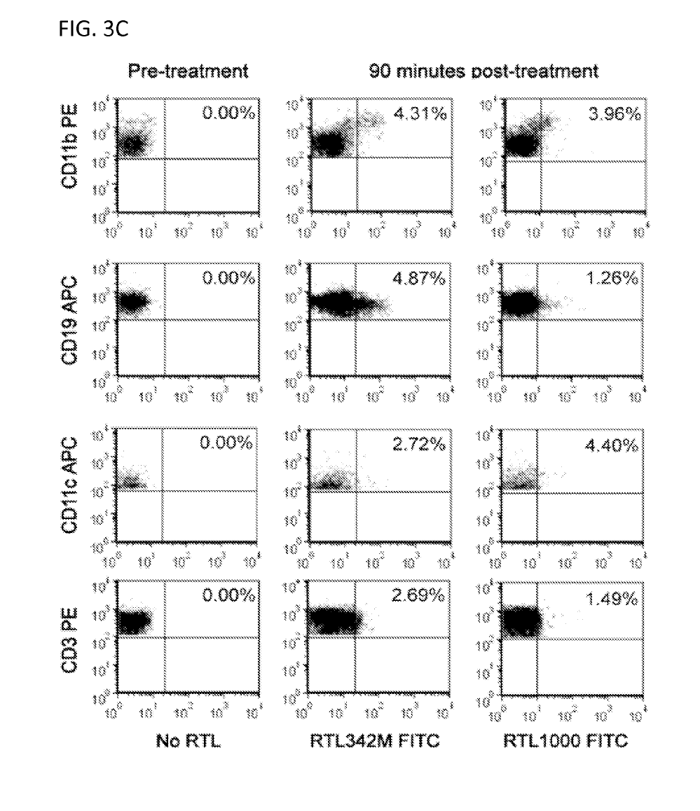

The major RTL-binding cell population detected ex vivo was CD11b⁺ monocytes (FIG. 2B, top row; note accumulation of cells that are positive for both PE and FITC staining in the upper right quadrant), with only modest binding by CD19+ B-cells, CD3+ T-cells and CD11c+ DC (FIG. 2B). Binding was substantially similar for both mMOG and hMOG peptides (FIG. 2C). Only minimal RTL binding of these respective PBMC subpopulations was detected in the lymphocyte gate (FIG. 3A-C).

This pattern of cell binding by both RTL342M and RTL1000 was validated in vitro after 1 hour incubation with PBMC from DR*1501-Tg mice. Similar to the results shown in FIG. 2B, the population that most bound to the pMHC complexes in vitro was CD11b+ monocytes, with minimal binding to CD19+ B cells, CD3+ T cells, and CD11c+ DCs (FIG. 2C). Direct binding of labeled RTL342M to GFP⁻ labeled CD11b⁺ monocytes was visualized by color enhanced fluorescence microscopy (FIG. 2D) and demonstrated cell surface as well as internalized complexes of RTL.

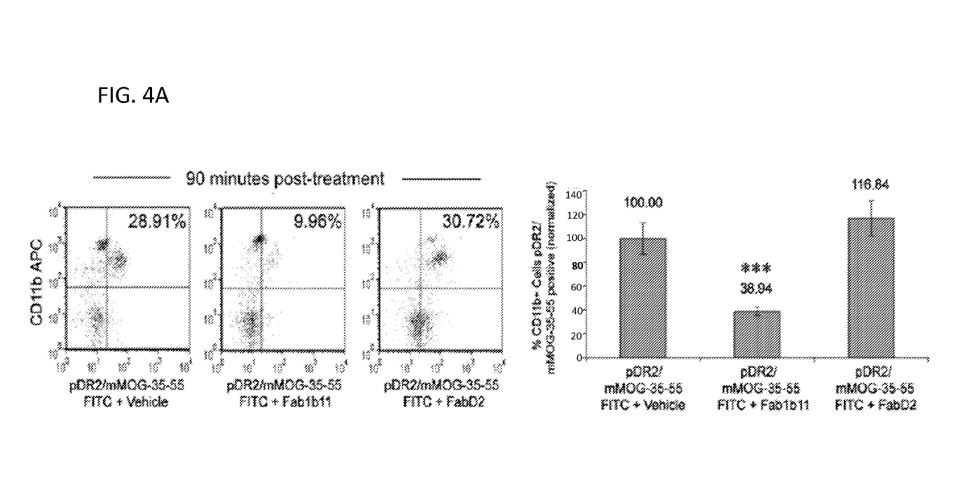

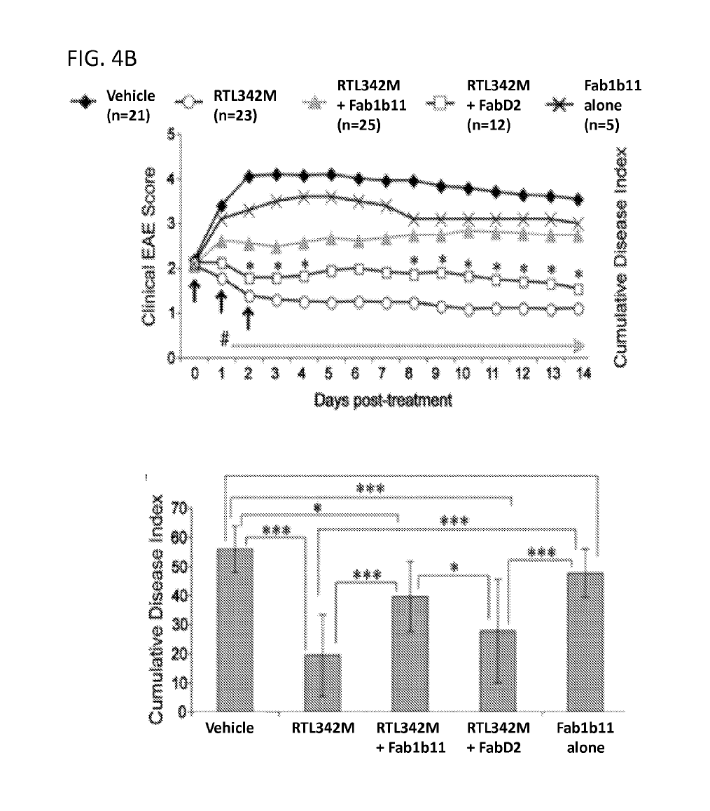

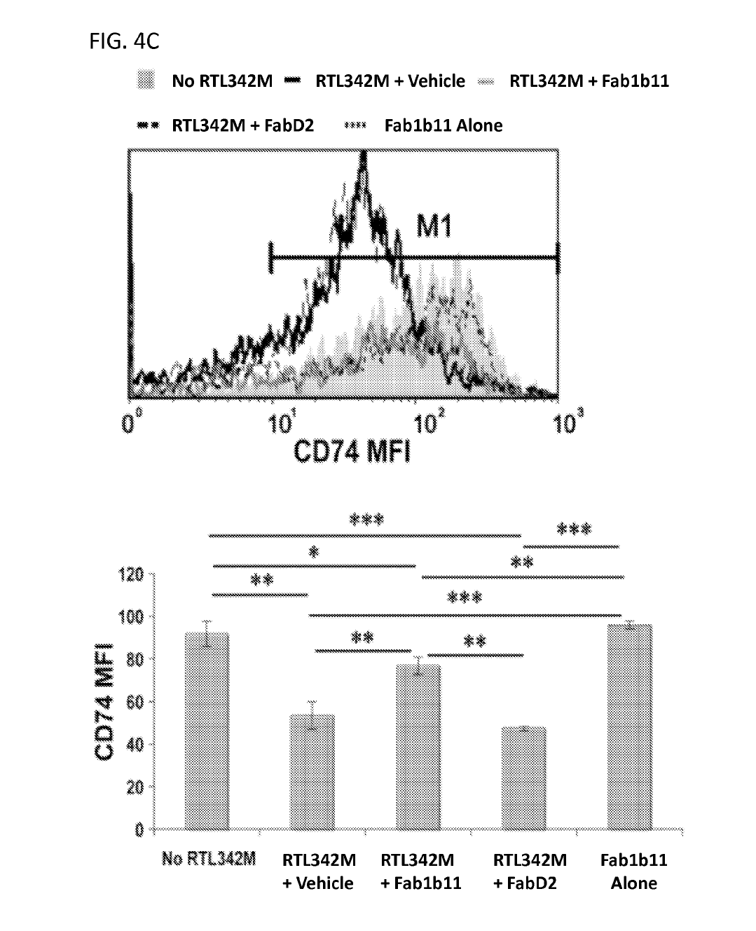

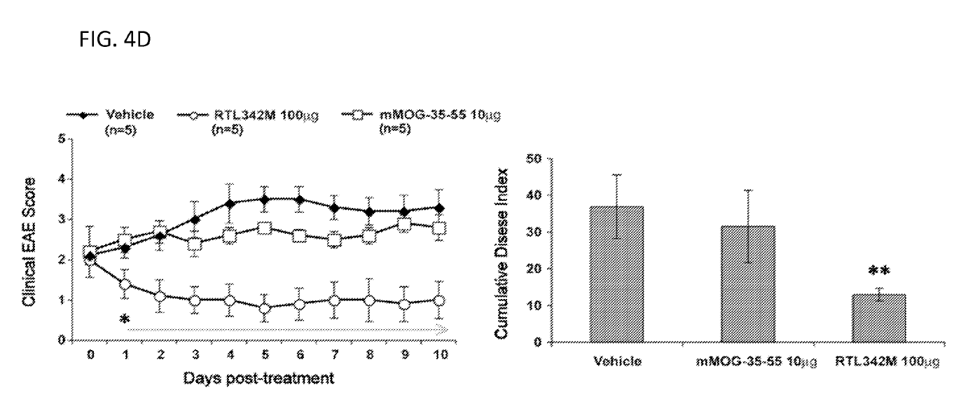

The Fab fragment of the 1B11 antibody binds to the two-domain pMHC molecules. Fab1B11 was tested for its ability to inhibit cell binding and neutralize RTL treatment effects in the EAE model. A control Fab fragment from the D2 antibody, which binds pDR/GAD555-567 (Dahan et al., Eur. J. Immunol. 41:1465-1479, 2011), was also utilized. Binding of pDR2/mMOG-35-55 to CD11b⁺ monocytes was inhibited in the presence of Fab1B11 by about 60% (p<0.0001) (FIG. 4A). Binding of pDR2/mMOG-35-55 to CD11b+ monocytes was increased by about 20% in the presence of FabD2, but this result was not statistically significant. Similarly, incubation of pDR2/mMOG-35-55 with Fab1B11 but not FabD2 prior to injection into DR*1501-Tg mice with EAE resulted in about 60% neutralization of the protective activity of pDR2/mMOG-35-55 (p<0.0001, FIG. 4B).

The Fab 1B11 antibody was also tested for its ability to block RTL-induced down-regulation of CD74. Incubation of RTL342M with Fab 1B11, but not FabD2 at a 1:1 or 1:2 molar ratio for two hours resulted in a 60% blockade of RTL342M-induced down-regulation of CD74 expression on CD11b+ monocytes in vitro (p<0.01) (FIG. 4C).

In a related study, 5 daily injections of free mMOG-35-55 peptide at an equimolar concentration as RTL342M (10 μg vs. 100 μg, respectively) did not produce significant inhibition of EAE compared to RTL342M (FIG. 4D and Table 6), thus confirming that the inhibitory activity was not mediated by the MOG peptide and that the neutralizing effect of Fab1B11 was directed against the DR2-β1α1 moiety rather than mMOG-35-55. These combined selective effects of Fab1B11 vs. FabD2 demonstrate that RTL and DRα1 binding and downregulation of CD74 is necessary for its therapeutic activity.

TABLE 6
Treatment of EAE with free mMOG-35-55 peptide (no RTL)

| | Inci- | | | Mor- | |
|---|---|---|---|---|---|
| Group | dence | Onset | Peak | tality | CDI |
| Vehicle | 5/5 | 11.2 ± 1.1 | 3.7 ± 1.0 | 0 | 36.9 ± 8.6 |
| mMOG-35-55 | 5/5 | 11.2 ± 1.2 | 3.0 ± 0.9 | 0 | 31.5 ± 9.8 |
| peptide | | | | | |
| RTL342M | 5/5 | 11.6 ± 1.3 | 2.0 ± 0.0 | 0 | 13.0 ± 1.8 |

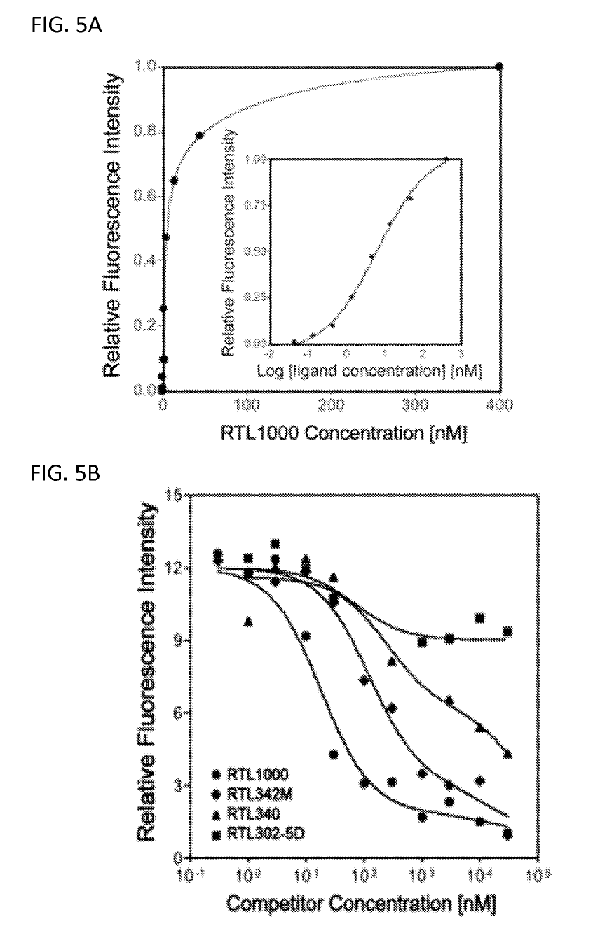

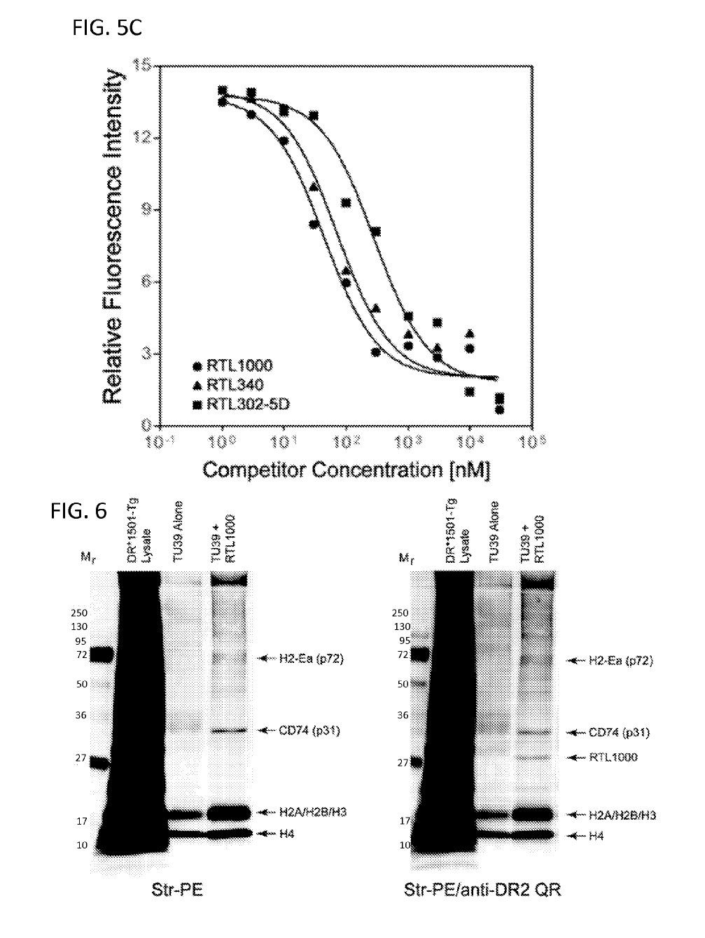

A saturation binding curve was established using 2 million spleen cells incubated for 1 hour on ice (to inhibit phagocytosis and low affinity non-specific binding) with increasing concentrations of Alexa-488-labeled RTL1000, followed by extensive washing. Cells with captured RTL1000 were centrifuged and the cell pellet solubilized in 6 M urea, with protein components separated by SDS-PAGE and fluorescence intensity of extracted Alexa-488-labeled RTL1000 quantified after gel electrophoresis. As shown in FIG. 5A, the saturation data best fit a hyperbolic two-site binding curve (R²=0.998), with one binding site for RTL1000 showing high affinity binding and rapid saturation with a K_{D} of 2.65 nM and another binding site showing lower affinity binding for RTL1000 with a K_{D} of 131 nM.

Competition experiments were then performed in which mixtures of Alexa488-labeled RTL1000 in combination with increasing concentrations of unlabeled RTL1000 were incubated with a fixed number of spleen cells. These competition experiments also demonstrated a two-site binding pattern of pDR2/hMOG35-55 to DR*1501-Tg splenocytes (FIG. 5B). The low affinity site had an EC₅₀ of 11 nM, indicating that a relatively low concentration of unlabeled RTL1000 was needed to displace labeled RTL1000. In contrast, the high affinity site had an EC₅₀ of 4,000 nM, indicating that a >350-fold higher concentration of unlabeled RTL1000 was needed to displace labeled RTL1000.

Additional competition studies were performed using A488-labeled RTL1000 mixed with increasing concentrations of other pDR2 constructs to evaluate the binding of both the MHC class II β1α1 and peptide moieties of pDR2/peptide. Although RTL1000, RTL342M, and RTL340 (pDR2/MBP-85-99) all could compete for both the low and high affinity binding sites (FIG. 5B and Table 7), “empty” pDR2/no peptide could compete with pDR2/hMOG-35-55 only for the low affinity site. These data clearly indicate that the low affinity binding site for RTL interacts with the β1α1 moiety even in the absence of a covalently-tethered peptide, whereas the high affinity binding site was much more likely to bind to a RTL when it included an antigenic peptide moiety, including a covalently tethered antigenic peptide moiety.

TABLE 7
EC₅₀ values of competitive binding of pDR2/peptides
with pDR2/hMOG-35-55 to DR*1501-Tg splenocytes

| | DR*1501-Tg | DR*1501-Tg | Class II deficient |
|---|---|---|---|
| Competitor vs. | splenocytes | splenocytes | mouse splenocytes |
| pDR2/hMOG- | EC₅₀ (nM) | EC₅₀ (nM) | EC₅₀ (nM) |
| 35-55 | High Affinity | Low Affinity | Low Affinity |
| RTL1000 | 4000 | 11 | 71.1 |
| RTL342M | 14900 | 122 | ND |
| RTL340 | 87000 | 140 | 43.2 |
| RTL302-5D | NA | 73 | 282 |

ND, not determined; NA, not applicable (only low affinity binding site detected)

Competition studies as described above were also performed using splenocytes from MHC class II deficient mice. As is shown in FIG. 5C and Table 7, only a low affinity binding site was present on MHC class II-KO splenocytes. These results implicated cell surface expressed MHC class II itself as the high affinity binding site for RTL.

TU39 monoclonal antibody alone or TU39-bound RTL1000 conjugated to Protein A was incubated overnight with biotinylated whole splenocytes in 1% CHAPS buffer. Bound membrane proteins were eluted and analyzed by electrophoresis and Western blotting. Biotinylated proteins were visualized with Streptavidin-PE. RTL1000 was visualized with anti-DR mAb HK14. This analysis revealed at least four distinct bands in addition to RTL1000 that were either not present (31 kD and 72 kD) or enriched (15 kD and 18 kD) compared with samples incubated without RTL1000 (FIG. 6). Parallel bands were eluted from an unstained sample and sequenced by LC-MS/MS (Table 8).

TABLE 8
Peptide sequences of proteins that bind pDR2/hMOG35-55
immunopurified from DR*1501-Tg mouse splenocytes.

| Protein | Peptide Sequences | % Coverage | SEQ ID NO: |
|---|---|---|---|
| p72: H2-E | 76 FASFEAQGAKANIAVDK(A) 92 | 16 | 86 |
| alpha chain | 69 RLEEFASFEAQGAKANIAVDK(A) 72 | | 87 |
| | 149 NGRPVTEGVSETVFLPR(D) 165 | | 88 |
| p31: CD74 | 62 LDKLTITSQNLQLESLR(M) 78 | 22 | 89 |
| | 179 NSLEEKKPTEAPPK(V) 191 | | 90 |
| | 256 EPLDMEDLSSGLGVTR(Q) 271 | | 91 |
| p18: Histone | 21 AGLQFPVGR(V) 30 | 33 | 92 |
| H2A | 83 HLQLAIR(N) 90 | | 93 |
| | 83 IILQLAIRNDEELNK(L) 97 | | 94 |
| | 101 VTIQGGVLPNIQAVLLPK(K) 119 | | 95 |
| | 101 VTIQGGVLPNIQAVLLPKK(T) 120 | | 96 |
| p18: Histone | 33 SRKESYSVYVYKVLK(Q) 47 | 50 | 97 |
| H2B | 59 AMGIMNSFVNDIFER(I) 73 | | 98 |
| | 94 EIQTAVRLLLPGELAK(H) 109 | | 99 |
| | 101 LLLPGELAKHAVSEGTK(A) 117 | | 100 |
| | 110 HAVSEGTKAVTKYTSSK 130 | | 101 |
| p18: Histone | 74 EIAKDFKTDLR(F) 84 | 24 | 102 |
| H3 | 101 LVGLFEDTNLCAIHAK(R) 116 | | 103 |
| | 124 DIKLAR(R) 129 | | 104 |
| p14: Histone | 26 DNIQGITKPAIR(R) 37 | 46 | 105 |
| H4 | 47 RISGLIYEETRGVLK(V) 61 | | 106 |
| | 81 KTVTAMDVVYALKR(Q) 94 | | 107 |
| | 98 TLYGFGG 104 | | 108 |

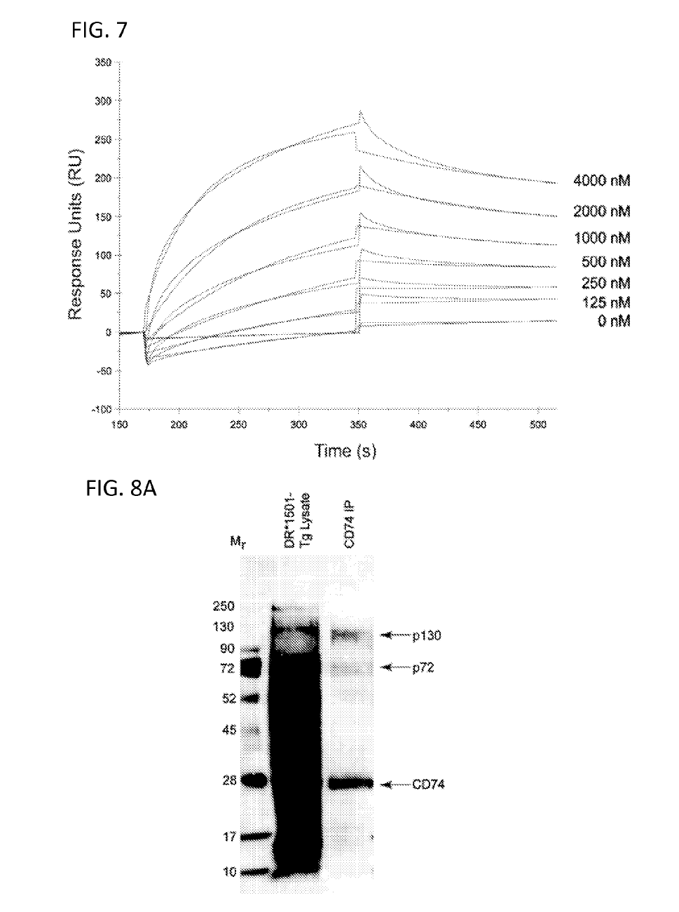

The major pDR2-binding proteins were identified as H4 histone (14 kD), H2A, H2B and H3 histones (18 kD), CD74 (31 kD) and MHC class II (72 kD). The p72 kD class II sequence was identified as an H-2Eα2 domain that was derived from the expressed DR2-transgene (Madsen et al., Nature Genetics 23:343-347, 1999), and not from the pDR2 construct. Further evaluations using plasmon surface resonance measurements confirmed low affinity binding interaction between RTL1000 and the histone complex (FIG. 7).

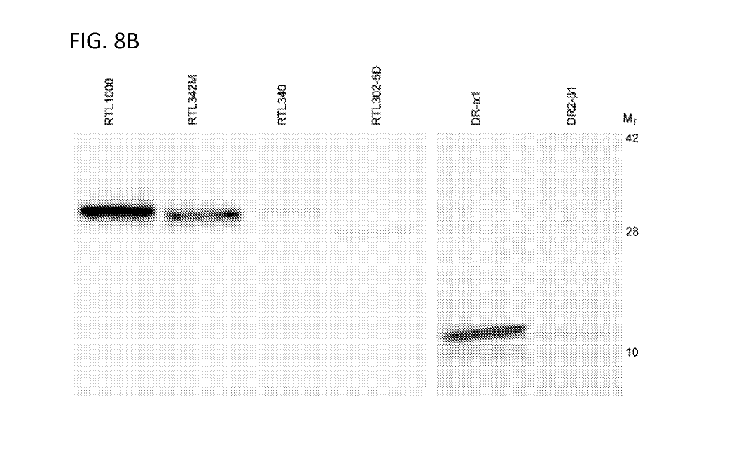

To confirm involvement of CD74 as a major component of the pMHC receptor, CD74 was immunoprecipitated from biotinylated DR*1501-Tg splenocyte membrane preparations using a Protein-L-conjugated anti-CD74 monoclonal antibody (ln-1) and visualized with Streptavidin-PE (FIG. 8A). Immunoprecipitated CD74 was found complexed with a p72 and p130 protein. No full-length MHC class II molecules were identified. Moreover, after incubating FITC-labeled RTL with bead-immunopurified CD74, both RTL1000 and RTL342M were readily coimmunoprecipitated with CD74. The RTL340 and RTL302-5D “empty” pDR2 also coimmunoprecipitated with CD74, but at much lower levels (FIG. 8B).

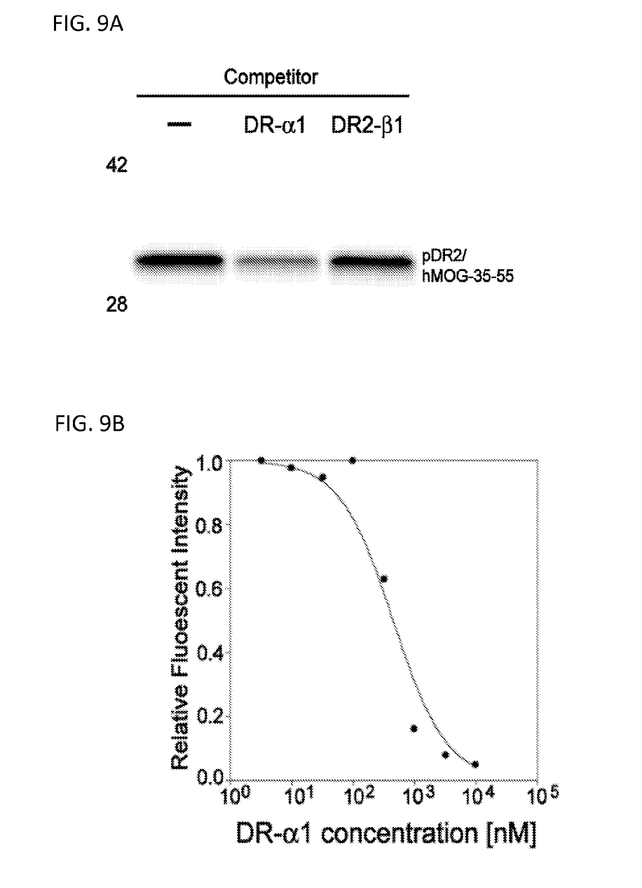

A construct comprising DR-α1 domain alone (with no DR2-β1 domain or peptide), coimmunoprecipitated with CD74 (FIG. 8B). This result was supported by competitive binding experiments showing that a DR-α1 domain alone competed with pDR2/hMOG-35-55 for binding to CD74 (FIG. 9A). A DR2-β1 domain (with no DR-α1 domain or peptide) did not coimmunoprecipitate with CD74 or compete with a pDR2/hMOG-35-55 for binding to CD 74 (FIG. 8B, FIG. 9A). These experiments established that there was a single binding site on CD74 for a DR-α1 domain without a DR2-β1 domain or peptide (FIG. 9B).

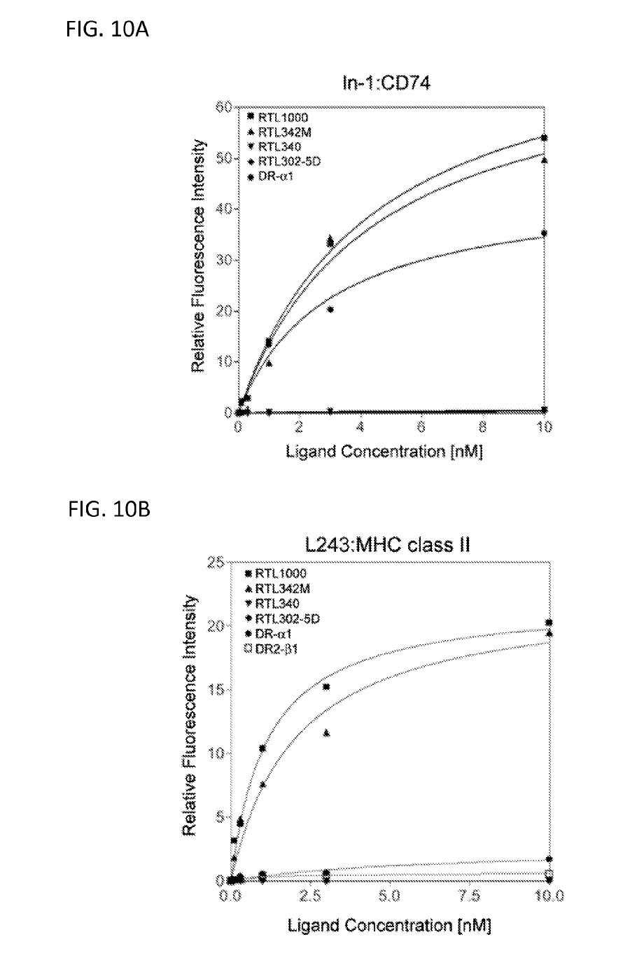

Direct binding of increasing concentrations of FITC-labeled RTL constructs to immune-purified CD74 (FIG. 10A) indicated binding of RTL1000, RTL-342M, and DR-α1 (but not other RTL constructs) to CD74 and that the binding was dose-dependent, saturable and involved a single binding site of about 2.9-4.5 nM (Table 9). Binding of FITC-labeled pDR2 constructs to four-domain MHC class II molecules in the same membrane preparation (after CD74-depletion) established binding of RTL1000 and RTL342M, but not the DR-α1 or other pDR2 constructs (FIG. 10B). The detected binding was dose-dependent, saturable and also involved a single binding site of about 1.1-2.0 nM (Table 9).

TABLE 9
Dissociation constants of binding of pMHC
complexes to CD74 and MHC class II

| | CD74 | MHC Class II |
|---|---|---|
| | One binding site | One binding site |
| | K_{D} (nM) | K_{D} (nM) |
| RTL1000 | 4.473 | 1.137 |
| RTL342M | 4.342 | 2.041 |
| RTL340 | NB | NB |
| RTL302-5D | NB | NB |
| DR-α1 | 2.948 | NB |
| DR2-β1 | NB | NB |

NB, no binding
For ligands that bound to CD74 and MHC class II, the R² was ≧0.95

These data indicated the presence of a single binding site on CD74 for a determinant in the DR-α1 moiety of the pDR2 molecule and a separate, slightly higher affinity binding site on 4-domain class II not present on the DR-α1 domain that may include the covalently-tethered peptide itself or alternatively, a structurally sensitive binding site on pDR2 that forms when an antigenic peptide, such as a covalently-tethered antigenic peptide is present.

These data further indicate that an isolated and purified DR-α1 without a DR2-β1 domain or peptide can bind to CD74.

### Example 4

### CD74 Expression Levels

This example describes the effects of pMHC binding on CD74 expression.

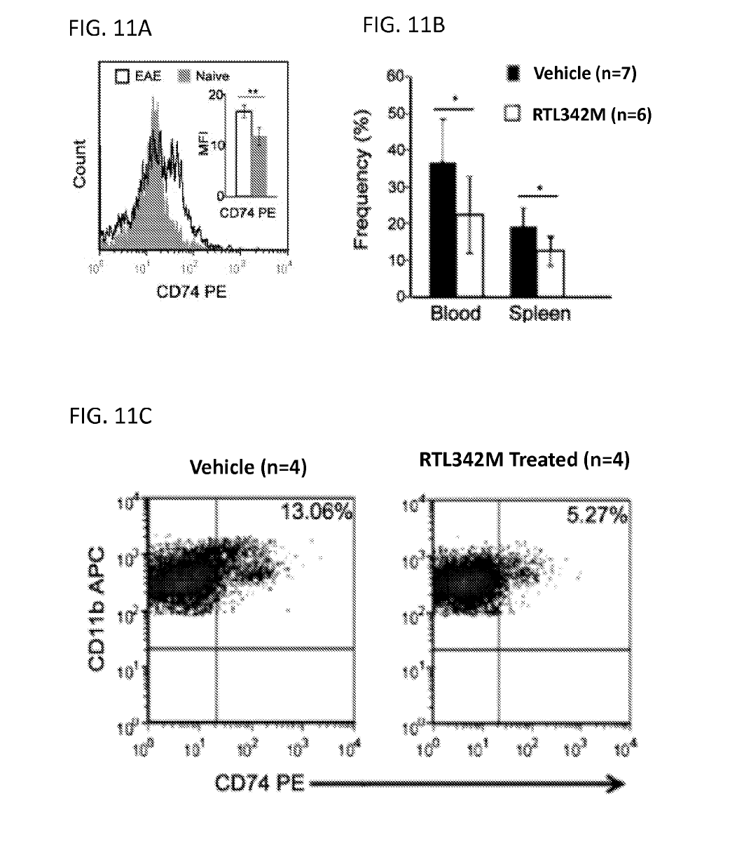

Ex vivo studies demonstrated that expression of CD74 on CD11b⁺ monocytes was increased in mice with EAE relative to naïve controls (FIG. 11A). Treatment of EAE mice with RTL342M at onset of clinical signs produced a significant down-regulation of CD74 expression within 48 hours. This result was observed on CD11b⁺ cells isolated from blood, spleen and spinal cords (FIGS. 11B and 11C). Treatment with pDR2/mMOG-35-55 did not alter MHC class II cell surface expression. This is in contrast with the literature, which suggests that MHC class II is generally not associated with CD74 on the monocyte cell surface (Roche et al., Proc. Natl. Acad. Sci. USA 90:8581-8595, 1993; Eynon et al., J. Biol. Chem. 274:26266-26271, 1999). Down-regulation of CD74 expression was not due to the blocking of the anti-CD74 binding site being blocked by RTL since CD74 could be immunoprecipitated from biotinylated DR*1501-Tg splenocyte membrane preparations that were pre-incubated with RTL342M or DR-α1 using Protein L conjugated anti-CD74 ln-1 mAb. (FIG. 20).

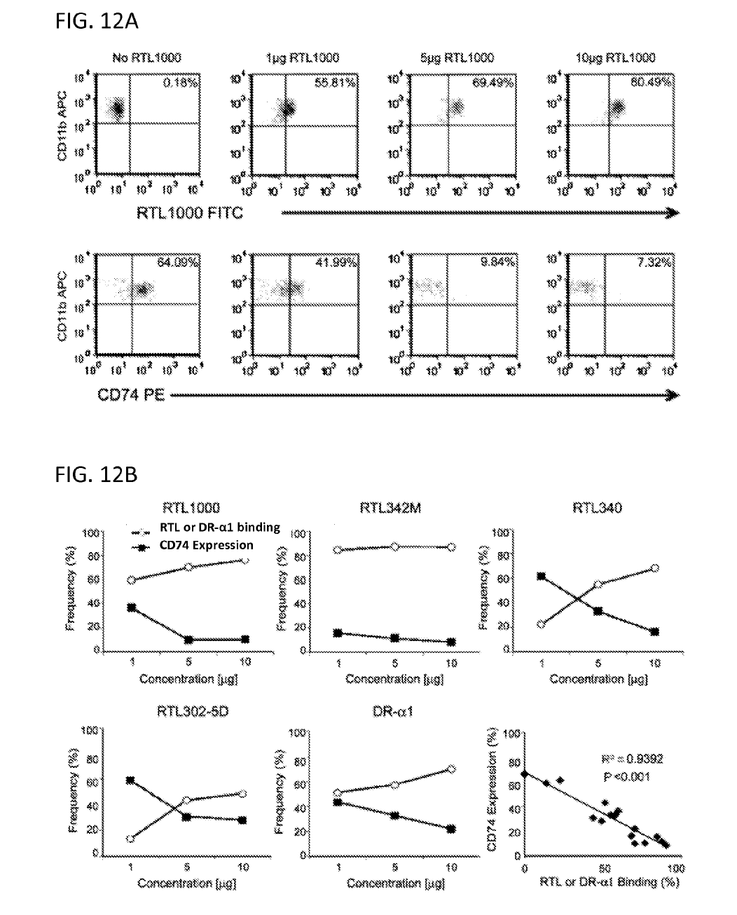

Naïve CD11b⁺ monocytes were incubated with increasing concentrations of RTL constructs for 1 hour at 37° C. and then evaluated by FACS for binding of RTL and cell-surface expression of CD74, as shown for RTL1000 (FIG. 12A). Results from five different constructs (FIG. 12B) demonstrated decreased expression of CD74 as a function of increasing RTL binding. This relationship was statistically significant when data points for all constructs were combined (p<0.001. FIG. 12B lower right panel). Note the inverse relationship between pMHC concentration and CD74 expression for DR-α1 alone.

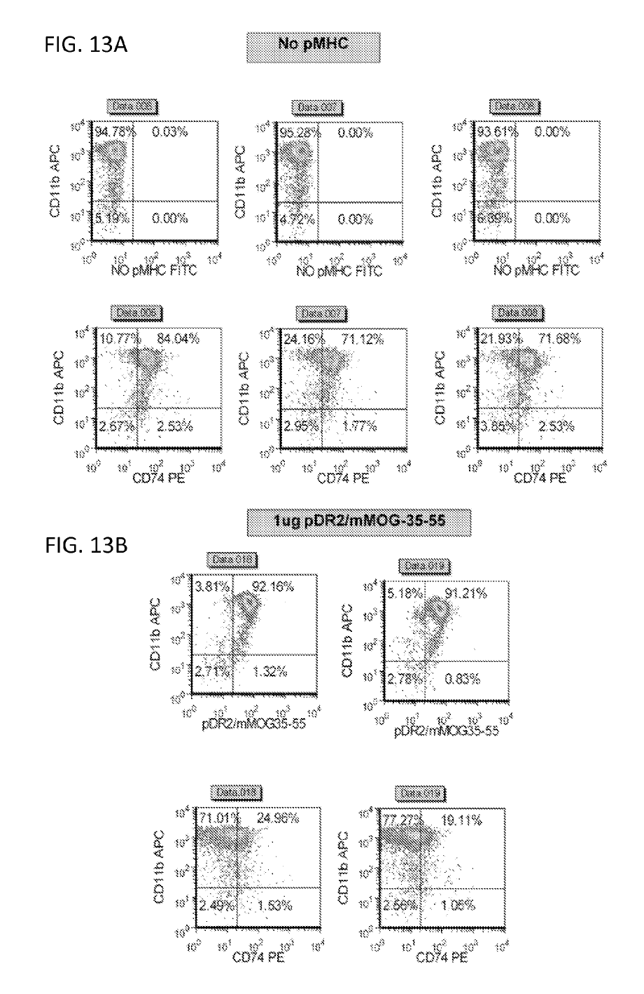

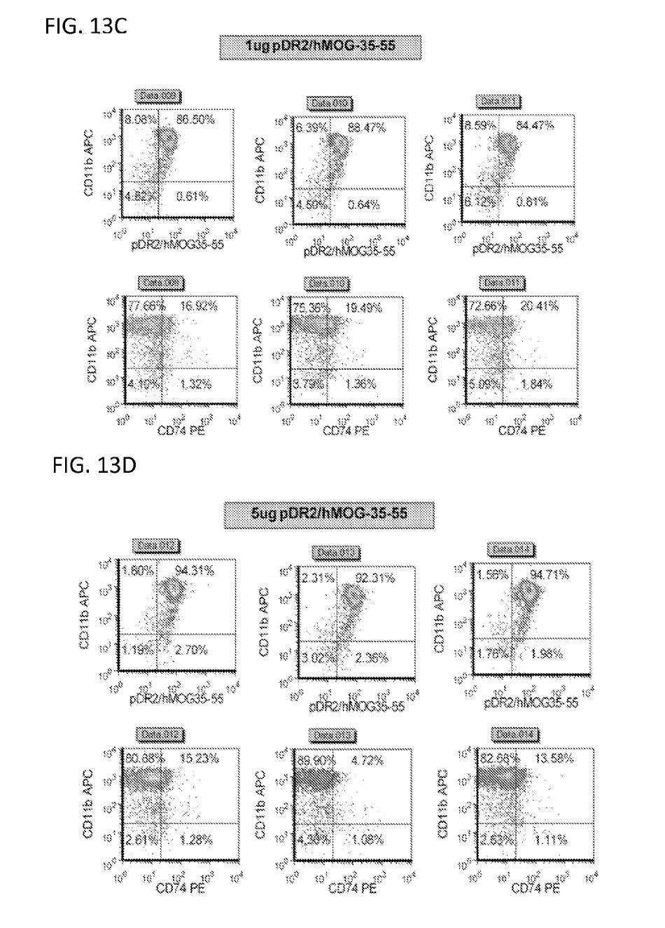

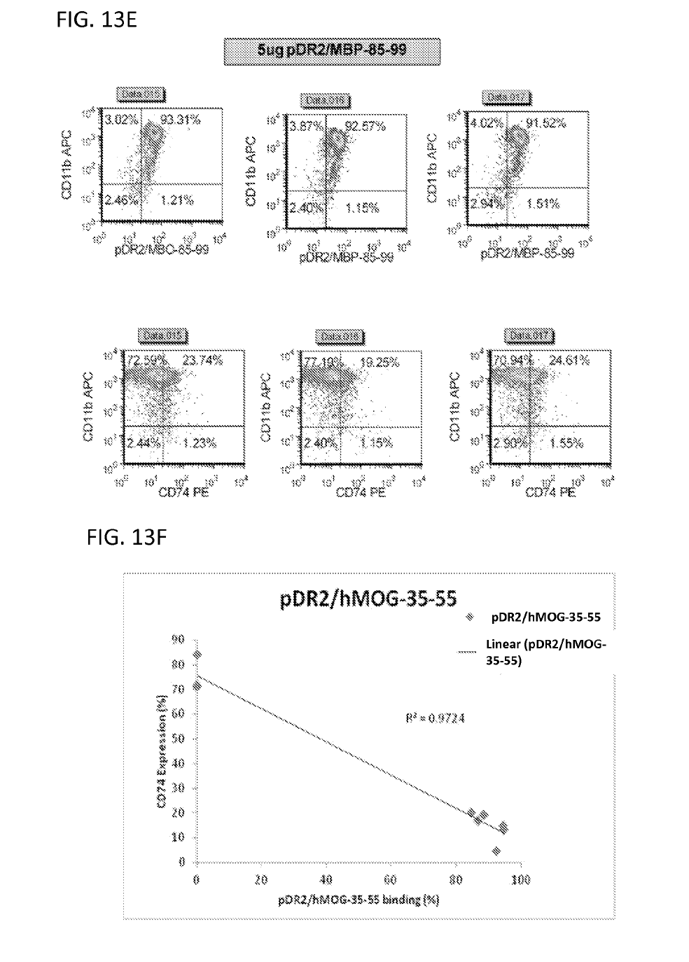

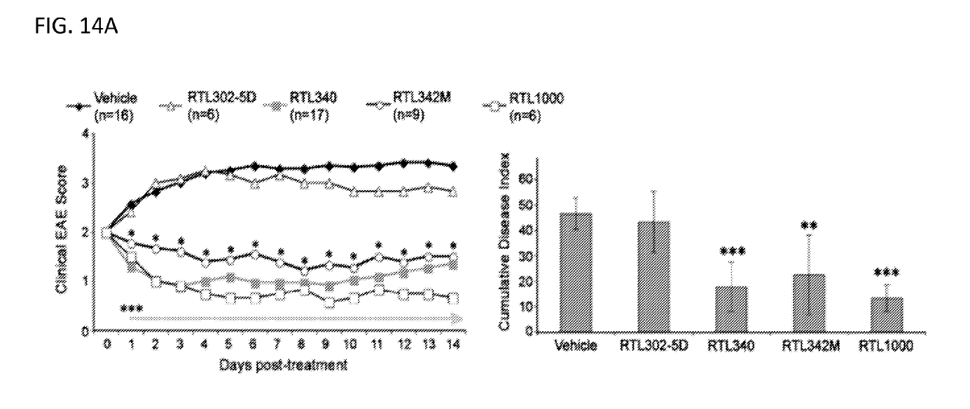

Binding of RTL1000 to human monocytes was also assessed. Human CD11b⁺ monocytes were isolated from a DR2⁺ human donor and treated with 1 μg RTL342M, 1 μg RTL1000, 5 μg RTL1000, 5 μg RTL340, and vehicle alone (FIGS. 13A-E). A regression analysis of the results from all treatment conditions with regard to the percentage of cells expressing CD74 and the percentage of cells binding FITC-RTL1000 following treatment is shown in FIG. 13F. The percentage of cells expressing CD74 inversely correlated with the percentage of cells that bind to labeled RTL1000.

### Example 5

### Effect of RTL Constructs on EAE

This example describes the effect of pMHC constructs on EAE, including the effect of DR-α1 alone.

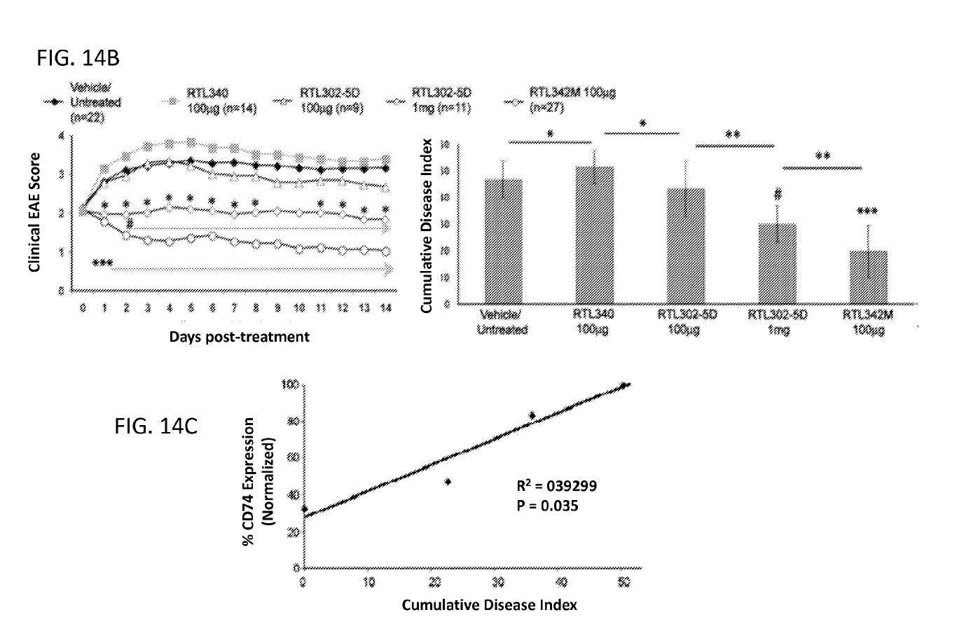

RTL1000 or RTL342M were compared with RTL340, pDR2/no peptide or vehicle for their ability to treat EAE induced with a non-cognate peptide. EAE was induced in MBP-TCR/DR2-Tg mice with MBP-85-99 peptide/CFA/Ptx. As is shown in FIG. 14A and Table 10, both RTL1000 and RTL342M showed effects on EAE comparable to that of pDR2/MBP-85-99 (bearing the cognate peptide but with less potent modulation of CD74). In contrast, “empty” pDR2/no peptide was ineffective at treating EAE and had weak effects on CD74 expression.

TABLE 10
Effect of pMHC constructs on EAE induced by MBP-85-99

| | Inci- | | | Mor- | |
|---|---|---|---|---|---|
| Group | dence | Onset | Peak | tality | CDI |
| Vehicle | 16/16 | 14.8 ± 4.6 | 3.7 ± 0.7 | 0 | 46.9 ± 7.5 |
| RTL302-5D | 6/6 | 17.5 ± 5.0 | 3.7 ± 0.8 | 0 | 43.3 ± 12.0 |
| RTL340 | 17/17 | 14.3 ± 3.8 | 2.3 ± 0.5 | 0 | 17.0 ± 9.0 |
| RTL342M | 9/9 | 16.3 ± 2.0 | 2.5 ± 1.0 | 0 | 22.6 ± 15.6 |
| RTL1000 | 6/6 | 19.7 ± 4.0 | 2.0 ± 0.0 | 0 | 13.3 ± 5.3 |

To explore further the possible peptide-independent inhibitory effects of pDR2 constructs on EAE, a 10-fold-higher dose of pDR2/no peptide (1 mg X2 for 5 days), which showed significant binding and modulation of CD74, was tested for therapeutic activity in DR2-Tg mice with mMOG-35-55-induced EAE compared to the standard dose and treatment regimen (100 μg X 5) of pDR2/no peptide, pDR2/mMOG-35-55, and vehicle. As shown in FIG. 14B and Table 11, treatment of EAE with the 1 mg dose of pDR2/no peptide indeed resulted in significant reduction of daily and cumulative EAE scores compared to treatment with vehicle. Treatment with the standard dose of RTL342M daily for either 2 or 5 days resulted in even more potent inhibition of EAE.

CD74 levels were assessed on CNS-derived CD11b⁺ cells from naïve DR2-Tg mice (background levels of CD74 with no clinical EAE) and mMOG-35-55 immunized DR2-Tg mice (DR*1501 Tg) treated with RTL342M, 100 μg pMHC daily X2), “empty” RTL302-5D (1 mg X2), or Vehicle (maximal disease-induced levels of CD74 with no treatment effect) 15 days after induction of EAE. As is shown in FIG. 14C, there was a significant relationship (p=0.035) between CD74 levels on CD11b+ cells and the average CDI for the different groups of mice.

TABLE 11
Effect of pMHC constructs on EAE induced by mMOG-35-55

| | Inci- | | | Mor- | |
|---|---|---|---|---|---|
| Group | dence | Onset | Peak | tality | CDI |
| Vehicle/ | 17/17 | 11.2 ± 1.4 | 3.6 ± 0.5 | 0 | 47.0 ± 7.5 |
| Untreated | | | | | |
| pDR2/ | 14/14 | 11.0 ± 0.0 | 3.9 ± 0.5 | 0 | 51.2 ± 6.2 |
| MBP-85-99 | | | | | |
| 100 μg x 5 | | | | | |
| pDR2/no | 9/9 | 11.7 ± 1.1 | 3.3 ± 0.9 | 0 | 43.1 ± 10.6 |
| peptide | | | | | |
| 100 μg x 5 | | | | | |
| pDR2/no | 3/3 | 9.0 ± 0.0 | 2.8 ± 0.3 | 0 | 34.2 ± 6.2 |
| peptide | | | | | |
| 1 mg x 2 | | | | | |
| pDR2/ | 3/3 | 9.7 ± 1.2 | 2.2 ± 0.3 | 0 | 21.6 ± 10.3 |
| mMOG-35-55 | | | | | |
| 100 μg x 2 | | | | | |
| pDR2/ | 14/14 | 11.3 ± 0.6 | 2.2 ± 0.3 | 0 | 17.1 ± 9.6 |
| mMOG-35-55 | | | | | |
| 100 μg x 5 | | | | | |

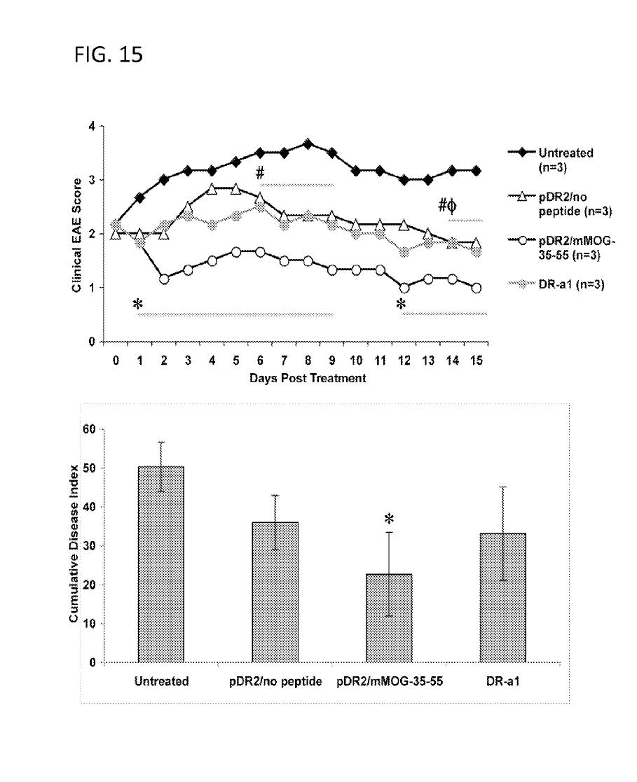

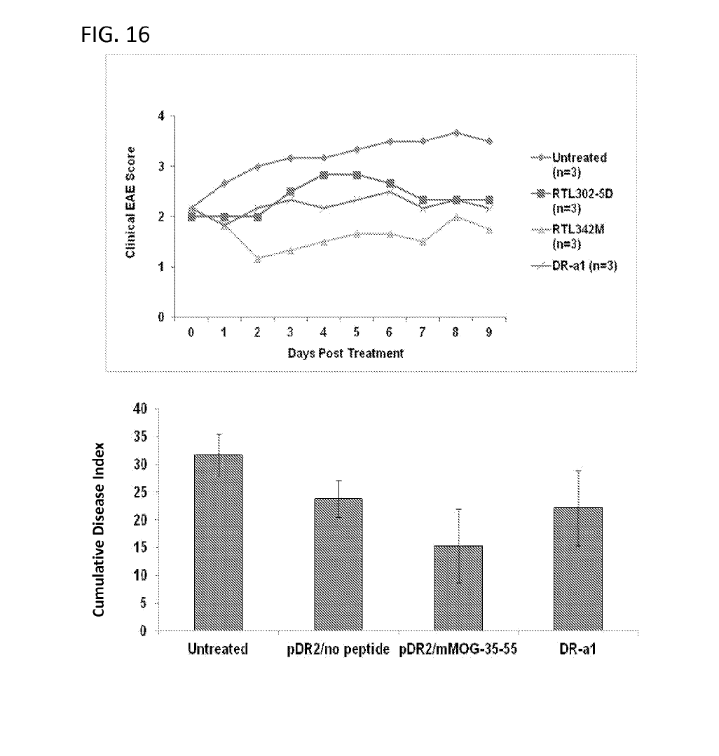

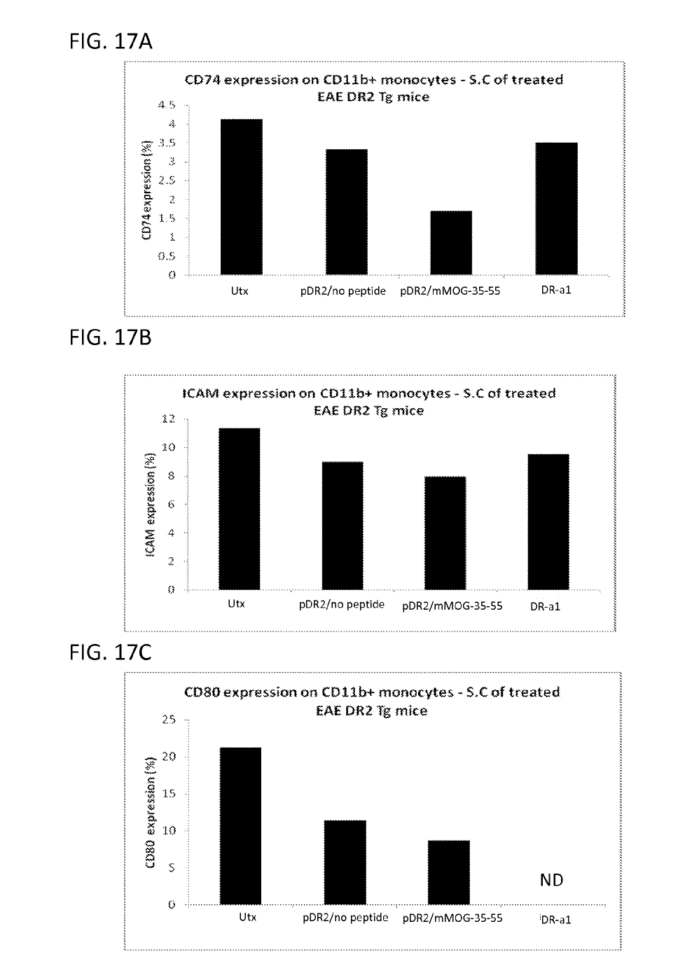

EAE was induced in mice by immunization with mMOG-35-55. At disease onset, the mice were treated with one of the following constructs: (i) pDR2 with covalently bound mMOG-35-55 (100 μg), (ii) pDR2 without peptide (1000 μg), (iii) DR-α1 (750 μg), or (iv) vehicle alone. The constructs were administered subcutaneously for two sequential days. On day 15 post-immunization, mice were sacrificed and spinal cords were harvested and pooled for each group. Isolated cells were stained for CD74, CD11b, CD80, ICAM, and HLA-DR expression and analyzed by FACS. Clinical scores for mice treated with pDR2 without peptide and with DR-α1 were lower than those treated with vehicle alone (FIG. 15). Similar results were obtained in mice treated with vehicle, RTL302-5D (pDR2 with 5 amino acid substitutions having decreased aggregation in solution), RTL342M (RTL302-5D with mMOG35-55), or DR-α1 (FIG. 16). Though the results do not rise to the level of significance, these results suggest that statistically significant results could be obtained with a larger sample size or by altering the dosing and/or administration parameters to achieve an optimal treatment regimen using pDR2 (no peptide) or DR-α1 to treat EAE.

CD74 expression was decreased in CD11b+ monocytes from spinal cord of mice treated with pDR2/mMOG-35-55 (FIG. 17A). CD80 expression was decreased in mice treated with pDR2/no peptide and pDR2/mMOG-35-55 (FIG. 17B). ICAM expression was not significantly changed in any treatment group as compared to untreated controls (FIG. 17C).

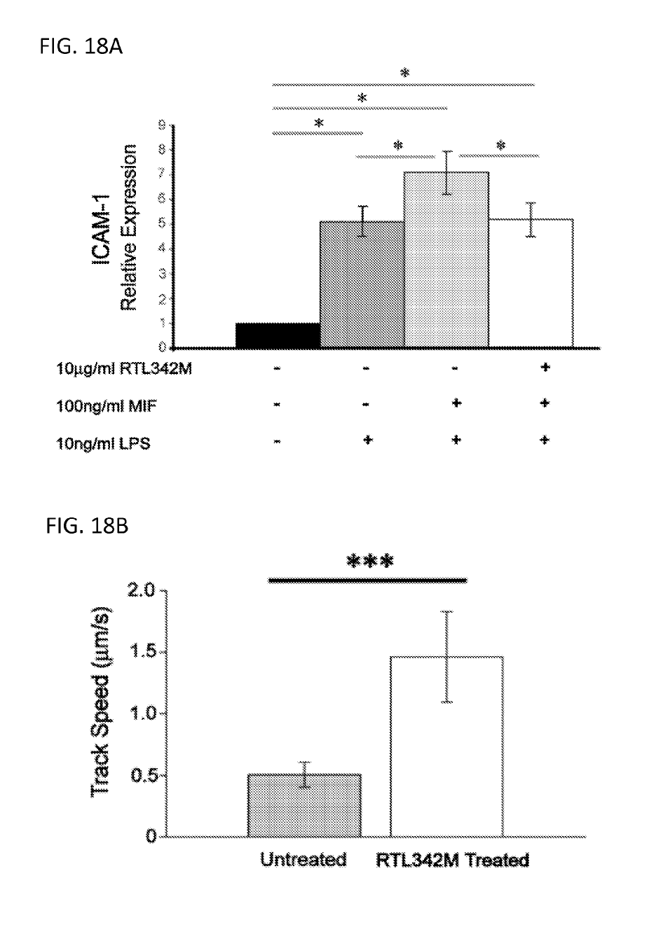

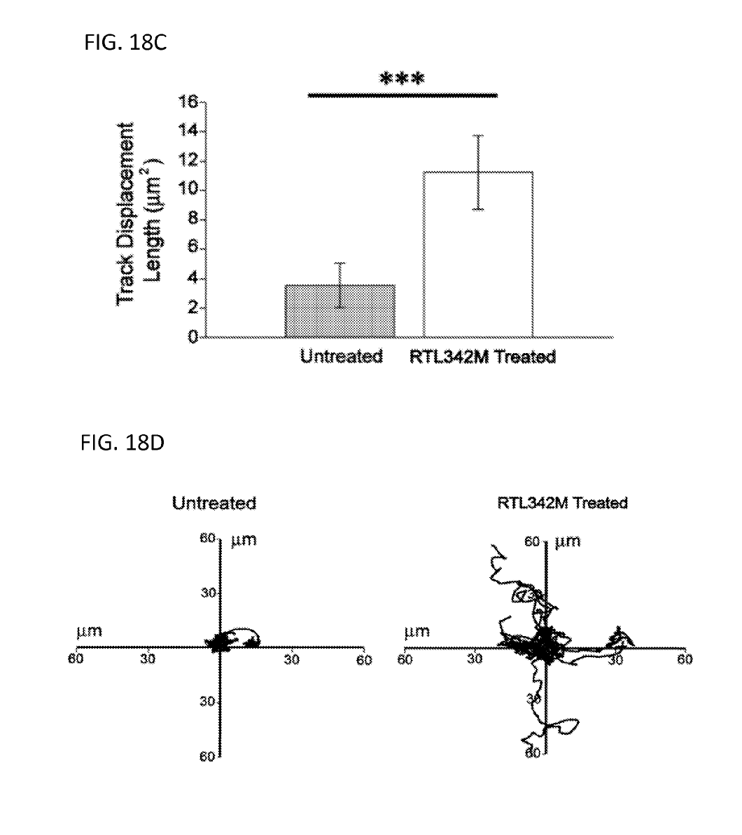

To establish if pMHC binding directly inhibited MIF-induced signaling, naïve splenocytes were incubated with pDR2/mMOG-35-55 for 1 hour prior to stimulation with MIF and LPS and then evaluated for expression of ICAM-1. As is shown in FIG. 18A, pDR2/mMOG-35-55 significantly reduced MIF-enhanced induction of ICAM-1 message to LPS background levels, demonstrating complete inhibition of the MIF-dependent effects but no further effect on LPS-dependent activation.

Binding of MIF to CD74 is known to inhibit random migration of macrophages. To determine if reduced CD74 cell-surface expression mediated by pDR2/mMOG-35-55 binding altered the migration pattern of monocytes, movement of GFP⁺CD11b⁺ cells isolated from naïve DR*1501/GFP-Tg mice was tracked for 2 hours in vitro with or without added pDR2/mMOG-35-55 using live imaging microscopy. As is shown in FIGS. 18B-D, significant increases were observed in measured speed (p<0.0005, FIG. 18B), mean square displacement (p<0.0005, FIG. 18C) and random migration path (FIG. 18D) in the presence of pDR2/mMOG-35-55. These data suggest that reduced cell surface expression of CD74 induced by pDR2/mMOG-35-55 is associated with reduced expression of ICAM-1 and increased random migration of CD11b+ monocytes, consistent with the blockade of MIF effects.

### Example 7

### Dose-Response of DRα1 in EAE

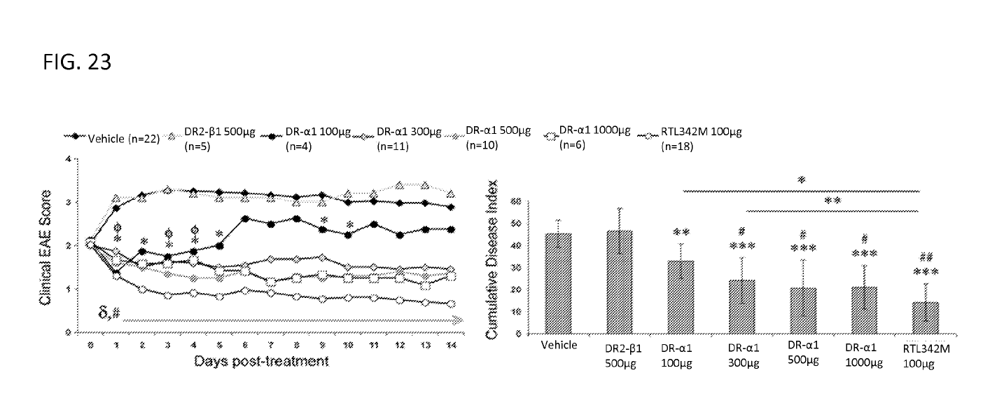

EAE was induced in mice by immunization with mMOG-35-55. At disease onset, the mice were treated with one of the following: (i) vehicle alone, (ii) DR2-β1 at 500 μg, (iii) DR-α1 at 100 μg, (iv) DR-α1 at 300 μg (v) DR-α1 at 500 μg, (vi) DR-α1 at 1000 μg, and RTL342M (DR2/mMOG35-55) at 100 μg. All dosages were daily X2. Results are shown in FIG. 23 and Table 12.

TABLE 12
Effects of different dosages of DRα1 construct on EAE

| | Inci- | | | Mor- | |
|---|---|---|---|---|---|
| Group | dence | Onset | Peak | tality | CDI |
| Vehicle | 22/22 | 10.9 ± 2.3 | 3.5 ± 0.6 | 0 | 45.3 ± 6.2 |
| DR2-β1 500 μg | 5/5 | 9.0 ± 0.0 | 3.6 ± 8.2 | 0 | 46.5 ± 10.2 |
| DR-α1 100 μg | 4/4 | 10.0 ± 1.2 | 2.6 ± 0.5 | 0 | 32.9 ± 7.8 |
| DR-α1 300 μg | 11/11 | 9.5 ± 0.8 | 2.3 ± 0.6 | 0 | 24.1 ± 10.3 |
| DR-α1 500 μg | 10/10 | 9.6 ± 0.7 | 2.5 ± 0.7 | 0 | 20.7 ± 12.7 |
| DR-α1 1000 μg | 6/6 | 10.7 ± 1.0 | 2.3 ± 0.4 | 0 | 21.1 ± 9.9 |
| RTL342M 100 μg | 18/18 | 10.4 ± 1.7 | 2.1 ± 0.2 | 0 | 14.2 ± 8.4 |

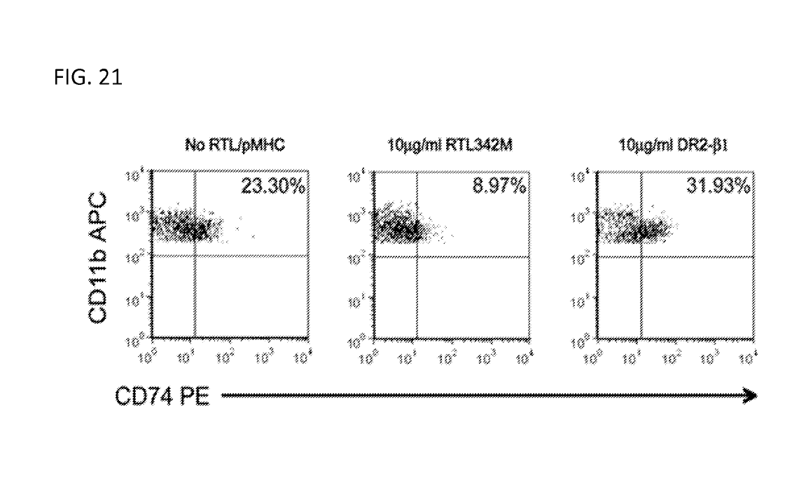

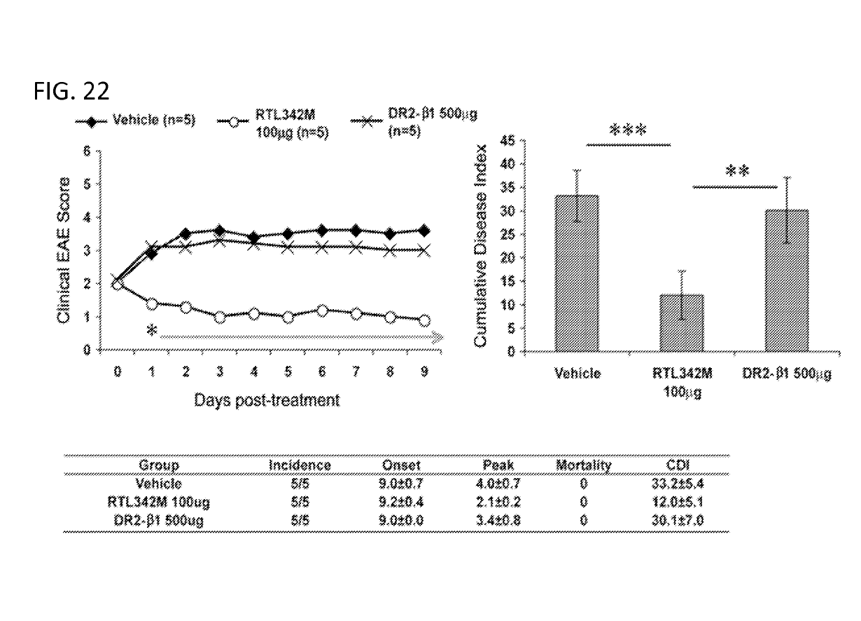

Additionally, mice treated with DR2-β1 alone did not downregulate CD74 or treat EAE. DR*1501-TG mice with mMOG35-55/CFA/Ptx-induced EAE were treated at the onset of clinical signs with 100 pg RTL342M, 500 μg DR2-β1 or vehicle daily for three days. Data are summarized in FIG. 21 and FIG. 22 and Table 13.

TABLE 13
Effect of DR2-β1 on EAE induced by mMOG-35-55

| | Inci- | | | Mor- | |
|---|---|---|---|---|---|
| Group | dence | Onset | Peak | tality | CDI |
| Vehicle | 5/5 | 9.0 ± 0.7 | 4.0 ± 0.7 | 0 | 33.2 ± 5.4 |
| RTL342M 100 μg | 5/5 | 9.2 ± 0.4 | 2.1 ± 0.2 | 0 | 12.0 ± 5.1 |
| DR2-β1 500 μg | 5/5 | 9.0 ± 0.0 | 3.4 ± 0.8 | 0 | 30.1 ± 7.0 |

### Example 8

### Methods of Treating or Inhibiting Multiple Sclerosis in a Subject

This example describes exemplary methods for treating or inhibiting multiple sclerosis in a subject. However, one of ordinary skill in the art will appreciate that methods that deviate from these specific methods can also be used to treat or inhibit multiple sclerosis in a subject. One of ordinary skill in the art can also modify these methods to treat a subject with a different disorder, such as a different autoimmune disorder.

Subjects having multiple sclerosis are selected. Subjects are treated weekly (for example, by subcutaneous injection) with an MHC class II α1 domain polypeptide (or portion thereof) or an MHC class II α1 polypeptide (or portion thereof) covalently linked to a myelin antigen (such as MOG 35-55, MBP 85-99, or PLP 139-151) at doses of 0.1 mg/kg to 50 mg/kg. Subjects are assessed for measures of multiple sclerosis including one or more of MRI measures (such as T₂ lesion load, volume of T₁ hypointensities, NAA levels, and whole brain atrophy); clinical measures (such as change in EDSS, change in SRS (Scripps Neurological Rating Scale), relapse rate, and 9-hole peg test), and immunologic measures (such as markers of Th1 and Th2 T cell lineages, FACS analysis of various T cell markers, cytokine production by T cells in vitro, and proliferation of T cells) prior to initiation of therapy, periodically during the period of therapy, and/or at the end of the course of treatment.

The effectiveness of therapy with an MHC class II α1 domain polypeptide to treat or inhibit multiple sclerosis (or another autoimmune disorder) in a subject can be demonstrated by an improvement in or decrease in progression of one or more symptoms of the disorder, for example, compared to a control, such as an untreated subject, a subject with the disorder prior to treatment (for example, the same subject prior to treatment), or a subject with the disorder treated with placebo (e.g., vehicle only).

### Example 9

### Methods of Determining Treatment Efficacy in a Subject

This example describes exemplary methods for determining efficacy of treatment with an MHC class II polypeptide in a subject. However, one of ordinary skill in the art will appreciate that methods that deviate from these specific methods can also be used to determine treatment efficacy in a subject.

A subject who has been treated with at least one dose of an MHC class II β1α1 polypeptide or an MHC class II α1 domain polypeptide (or portion thereof) is selected. A sample, such as a blood sample, is obtained from the subject. In some examples, expression of CD74 is determined in the sample, for example by extracting nucleic acids from the sample and measuring CD74 expression by RT-PCR or by determining CD74 protein expression by ELISA or flow cytometry. In other examples, CD74 activity is determined, for example by contacting the sample with MIF and measuring one or more of ERK1/2 activity, NF-κB activation, Bcl-2 expression, IL-8 secretion, cell proliferation, or apoptosis. The level of CD74 expression and/or activity is compared to a control (for example, a reference value or a CD74 expression or activity level determined in a sample from the subject before treatment with the MHC class II polypeptide).

The treatment is determined to be effective if the CD74 expression and/or activity level is less than or equal to the control. The treatment is determined to be suboptimally effective if the CD74 expression and/or activity level is greater than the control. The dosage of the MHC class II polypeptide can be maintained (or decreased) if the treatment is determined to be effective. The dosage of the MHC class II polypeptide can be increased if the treatment is determined to be suboptimally effective. One of ordinary skill in the art can select an appropriate dosage (such as an incremental increase or decrease in dosage), based on the CD74 expression or activity level, the disorder being treated, the condition of the subject and other factors.

### Example 10

### Methods of Optimizing Treatment Efficacy in a Subject

This example describes exemplary methods for optimizing efficacy of treatment with an MHC class II polypeptide in a subject. However, one of ordinary skill in the art will appreciate that methods that deviate from these specific methods can also be used to optimize treatment efficacy in a subject.

A subject having an autoimmune or inflammatory disorder (such as multiple sclerosis) is selected. The subject is administered a dosage of an MHC class II β1α1 polypeptide or an MHC class II α1 domain polypeptide (or portion thereof). A sample, such as a blood sample, is obtained from the subject. In some examples, expression of CD74 is determined in the sample, for example by extracting nucleic acids from the sample and measuring CD74 expression by RT-PCR or by determining CD74 protein expression by ELISA or flow cytometry. In other examples, CD74 activity is determined, for example by contacting the sample with MIF and measuring one or more of ERK1/2 activity, NF-κB activation, Bcl-2 expression, IL-8 secretion, cell proliferation, or apoptosis. The level of CD74 expression and/or activity is compared to a control (for example, a reference value or a CD74 expression or activity level determined in a sample from the subject before treatment with the MHC class II polypeptide). A dosage of the polypeptide to be subsequently administered to the subject is then determined. If the CD74 expression or activity level is greater than the control the dosage of the MHC class II polypeptide can be increased to improve treatment efficacy.

If the CD74 expression or activity level is less than or equal to the control the dosage of the MHC class II polypeptide can be maintained or decreased. One of ordinary skill in the art can select an appropriate dosage (such as an incremental increase or decrease in dosage), based on the CD74 expression or activity level, the disorder being treated, the condition of the subject and other factors.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. An isolated polypeptide comprising:
a major histocompatibility complex (MHC) class II α1 domain, wherein the polypeptide does not comprise an MHC class II α2, β1, or β2 domain; or
a portion of an MHC class II α1 domain, wherein the portion of the MHC class II α1 domain is capable of binding to CD74 or decreasing expression or activity of CD74.

2. The isolated polypeptide of claim 1, wherein the α1 domain comprises a human MHC class II α1 domain.

3. (canceled)

4. The isolated polypeptide of claim 1, wherein the MHC class II α1 domain comprises amino acid residues 1-75 of a mature MHC class II α chain, or at least 5 contiguous amino acids thereof.

5. The isolated polypeptide of claim 1, wherein the α1 domain comprises an α1 domain from an α chain comprising the amino acid sequence set forth in any one of SEQ ID NOs: 1-49.

6. The isolated polypeptide of claim 1, wherein the portion of the MHC class II α1 domain comprises amino acid residues 38-58 of a mature DR-α chain or a portion thereof, or a homologous region of DPα or DQα.

7. The isolated polypeptide of claim 1, further comprising an antigenic determinant.

8. The isolated polypeptide of claim 7, wherein the antigenic determinant is covalently linked to the α1 domain.

9. (canceled)

10. The isolated polypeptide of claim 7, wherein the antigenic determinant comprises a myelin protein peptide antigen, a celiac disease associated peptide antigen, a rheumatoid arthritis associated peptide antigen, a uveitis associated peptide antigen, or a type I diabetes associated peptide antigen.

11. The isolated polypeptide of claim 10, wherein the antigenic determinant is a myelin protein peptide antigen comprising a myelin oligodendrocyte glycoprotein (MOG) peptide, a myelin basic protein (MBP) peptide, or a proteolipid protein (PLP) peptide or a type I diabetes associated peptide antigen comprising insulin B:16-23.

12. The isolated polypeptide of claim 11, wherein the myelin protein peptide antigen comprises MOG35-55, MBP85-99, MBP149-171, or PLP139-151.

13. (canceled)

14. An isolated nucleic acid molecule encoding the polypeptide of claim 1.

15. A vector comprising the isolated nucleic acid molecule of claim 14 operably linked to a promoter.

16. A pharmaceutical composition comprising the isolated polypeptide of claim 1 and a pharmaceutically acceptable carrier.

17. A method of treating or inhibiting a disorder in a subject comprising administering an effective amount of the isolated polypeptide of claim 1 to the subject, thereby treating or inhibiting the disorder.

18. The method of claim 17, wherein the disorder comprises an inflammatory disorder, an autoimmune disorder, a retinal disorder, stroke, and/or substance addiction.

19. The method of claim 18, wherein the inflammatory and/or autoimmune disorder comprises multiple sclerosis, celiac disease, rheumatoid arthritis, type I diabetes mellitus, inflammatory bowel disease, ankylosing spondylitis, Goodpasture's syndrome, Hashimoto's thyroiditis, systemic lupus erythematosus, psoriasis, uveitis, optic neuritis and/or, wherein the substance addiction comprises amphetamine abuse.

20-23. (canceled)

24. The method of claim 17, wherein treating or inhibiting the disorder comprises decreasing expression or activity of CD74 as compared to a control.

25. The method of claim 17, further comprising determining CD74 expression or activity level in a sample from the subject.

26. The method of claim 24, wherein the CD74 expression level comprises CD74 RNA and/or protein expression level or wherein the CD74 activity level comprises NFκB activity, cell proliferation, apoptosis, or a combination of two or more thereof.

27-28. (canceled)

29. The method of claim 25, further comprising comparing the CD74 expression or activity level to a control and increasing dose or frequency of the isolated polypeptide if the CD74 expression or activity level is greater than the control.

30. A method of determining efficacy of treatment of a disorder in a subject with a polypeptide comprising an MHC Class II α1 domain, an MHC Class II β1 domain, or a combination thereof, wherein the polypeptide does not comprise an α2 domain or a β2 domain, comprising:
determining a CD74 expression or activity level in a sample from the subject;
comparing the CD74 expression or activity level to a control; and
determining the efficacy of treatment, wherein the treatment is considered to be effective if the CD74 expression or activity level is less than or equal to the control.

31. (canceled)

32. A method of treating or inhibiting a disorder in a subject comprising:
administering to the subject with the disorder a dosage of a polypeptide comprising an MHC Class II α1 domain, an MHC Class II β1 domain, or a combination thereof, wherein the polypeptide does not comprise an α2 domain or a β2 domain, wherein the dosage of the polypeptide is increased if CD74 expression or activity level in a sample from the subject is greater than a control or the dosage of the polypeptide is decreased if CD74 expression or activity level in a sample from the subject is less than or equal to a control.

33-34. (canceled)

35. The method of claim 32, wherein the polypeptide comprises:
a major histocompatibility complex (MHC) class II α1 domain, wherein the polypeptide does not comprise an MHC class II α2, β1, or β2 domain;
a portion of an MHC class II α1 domain, wherein the portion of the MHC class II α1 domain is capable of binding to CD74 or decreasing expression or activity of CD74; or
an MHC Class II β1 and an MHC Class II α1 domain, wherein the amino terminus of the α1 domain is covalently linked to the carboxy terminus of the β1 domain and wherein the polypeptide does not comprise an α2 or β2 domain.

36-42. (canceled)

43. The method of claim 32, wherein the polypeptide further comprises an antigenic determinant.

44-45. (canceled)

46. The method of claim 43, wherein the antigenic determinant comprises a myelin protein peptide antigen, a celiac disease associated peptide antigen, a rheumatoid arthritis associated peptide antigen, a uveitis associated peptide antigen, or a type I diabetes associated peptide antigen.

47-50. (canceled)

51. The method of claim 32, wherein the disorder comprises an inflammatory and/or autoimmune disorder, a retinal disorder, stroke, or substance addiction.

52-53. (canceled)

54. The method of claim 32, further comprising administering a subsequent dose of the polypeptide to the subject, wherein the dose is adjusted based on the CD74 expression or activity level.

55. A method of decreasing CD74 expression or activity level in a cell, comprising contacting the cell with the polypeptide of claim 1.

56-57. (canceled)
